(19) United States
(12) Patent Application Publication
(10) Pub. No.: US 2024/0352095 A1
(43) Pub. Date: Oct. 24, 2024

(54) ARENAVIRUS MONOCLONAL ANTIBODIES AND USES

(71) Applicants: THE ADMINISTRATORS OF THE TULANE EDUCATIONAL FUND, New Orleans, LA (US); THE SCRIPPS RESEARCH INSTITUTE, La Jolla, CA (US); THE BOARD OF REGENTS OF THE UNIVERSITY OF TEXAS SYSTEM, Austin, TX (US); ZALGEN LABS, LLC, Germantown, MD (US)

(72) Inventors: Luis M. BRANCO, Germantown, MD (US); Robert F. GARRY, New Orleans, LA (US); James E. ROBINSON, New Orleans, LA (US); Erica O. SAPHIRE, La Jolla, CA (US); Kathryn M. HASTIE, La Jolla, CA (US); Thomas W. GEISBERT, Albany, TX (US)

(21) Appl. No.: 18/392,147

(22) Filed: Dec. 21, 2023

Related U.S. Application Data
(62) Division of application No. 17/520,338, filed on Nov. 5, 2021, now abandoned.
Application No. 17/520,338 is a division of application No. 16/466,544, filed on Jun. 4, 2019, now Pat. No. 11,198,723.
(60) Provisional application No. 62/430,225, filed on Dec. 5, 2016.

(51) Int. Cl. A61P 31/14; C07K 16/10

(52) U.S. Cl. CPC A61P 31/14; C07K 16/10; C07K 2317/21; C07K 2317/31; C07K 2317/33; C07K 2317/565; C12N 2760/10011

(57) ABSTRACT

Disclosed herein are compositions comprising recombinant arenavirus monoclonal antibodies and antigen-binding fragments thereof, as well as therapeutic methods using the antibodies. In some embodiments, the antibodies provide pan-arenavirus protection against a number of arenavirus types and strains.

### CROSS REFERENCE TO RELATED APPLICATIONS

This application is a divisional of U.S. application Ser. No. 17/520,338, filed on Nov. 5, 2021, which is a divisional of U.S. application Ser. No. 16/466,544, filed on Jun. 4, 2019, now U.S. Pat. No. 11,198,723, which is a 371 of International Application No. PCT/US2017/064744, filed on Dec. 5, 2017, which claims the benefit of U.S. Provisional Application Ser. No. 62/430,225, filed on Dec. 5, 2016, each of which is incorporated herein by reference in its entirety.

### STATEMENT REGARDING FEDERALLY SPONSORED RESEARCH OR DEVELOPMENT

This invention was made, in part, with support provided by the United States government under Grant Nos. U19 AI109762, 1 R01 AI104621, R43 AI120472, and NIAID Project No. 272200900049C-0-0-1 from the National Institutes of Health. The government has certain rights in this invention.

### FIELD OF THE INVENTION

The present invention relates to novel arenavirus monoclonal antibodies, to compositions comprising the arenavirus monoclonal antibodies, and methods comprising the same.

### INCORPORATION OF SEQUENCE LISTING

Biological sequence information for this application is included in a XML file having the file name “TU-439 Div2.xml”, created on Dec. 19, 2023, and having a file size of 186,909 bytes, which is incorporated herein by reference.

### BACKGROUND

Lassa virus (LASV) and several other members of the Arenaviridae are classified as Biosafety Level 4 and NIAID Biodefense Category A agents. The present invention will fill a vital biodefense need for rapid multiagent immunodiagnostic assays for arenaviruses and for effective therapeutics against arenaviral disease, and will provide a major advance for public health management of an important family of viral pathogens. Several arenaviruses, chiefly Lassa virus (LASV) in West Africa, cause hemorrhagic fever (HF) disease in humans and pose serious public health concerns in their endemic regions. The global endemicity of the prototypic arenavirus lymphocytic choriomeningitis virus (LCMV) is not a causative agent of HF, but mounting evidence indicates that LCMV is a neglected human pathogen of clinical significance that can cause neurologic disease in the fetus, child and adult stages. In addition, LCMV poses a special threat in immune-compromised individuals, as tragically illustrated by recent cases of transplant-associated infections by LCMV with a fatal outcome in the United States and Australia. Moreover, the high seroprevalence of LCMV within different urban populations across the world, including the US, has raised the question of whether LCMV may contribute to the many cases of undiagnosed aseptic meningitis reported yearly.

Lassa fever. The most prevalent arenaviral disease is Lassa fever (LF), an often-fatal hemorrhagic fever named for the Nigerian town in which the first described cases occurred in 1969 (Buckley and Casals, 1970). Parts of Guinea, Sierra Leone, Nigeria, and Liberia are endemic for the etiologic agent, LASV (Birmingham and Kenyon, 2001). Although detailed surveillance of LASV is hampered by many factors, including the lack of a widely available diagnostic test, it is clear that the public health impact is immense. There are as many as 300,000 cases of Lassa per year in West Africa and 5,000 deaths (see the CDC website at www(dot)cdc(dot)gov/ncidod/dvrd/spb/mnpages/dispages/lassaf(dot)htm). In some parts of Sierra Leone, 10-16% of all patients admitted to hospitals have Lassa fever. Case fatality rates for Lassa fever have typically been reported as 15% to 20%, and as high as 45% during epidemics, with a recent multi-year study in Sierra Leone reporting a 69% rate (Schaffer et al., 2014). LASV has been associated with severe nosocomial outbreaks involving health care workers and laboratory personnel (Fisher-Hoch et al., 1995). The mortality rate for women in the last month of pregnancy is always high, about 90%, and LASV infection causes high rates of fetal death at all stages of gestation (Walls, 1985). Mortality rates for Lassa appear to be higher in non-Africans, which is of concern because Lassa is the most commonly exported hemorrhagic fever (Haas et al., 2003; Holmes et al., 1990).

Old and New World arenaviruses. Arenaviruses are enveloped viruses with a bi-segmented negative strand (NS) RNA genome. Each genomic RNA segment, L (ca. 7.3 kb) and S (ca. 3.5 kb), uses an ambisense coding strategy to direct the synthesis of two polypeptides in opposite orientation, separated by a non-coding intergenic region. The S RNA encodes the viral glycoprotein precursor (GPC) and the nucleoprotein (NP). GPC is co- and post-translationally processed to yield the two mature virion surface glycoproteins GP1 and GP2 that together with the stable signal peptide (SSP) form the GP complex that decorates the virus surface and directs virus cell entry via receptor-mediated endocytosis. The L RNA encodes the viral RNA dependent RNA polymerase (L polymerase), and the small RING finger protein Z that has functions of a bona fide matrix protein. The structure of arenavirus GP2 appears to be a class I fusion protein, which is common to envelope glycoproteins of myxoviruses, retroviruses and filoviruses (Gallaher, DiSimone, and Buchmeier, 2001). When viewed by transmission electron microscopy, the enveloped spherical virions (diameter: 110-130 nm) show grainy particles that are ribosomes acquired from the host cells (Murphy and Whitfield, 1975), hence the basis for the family name of the Latin word “arena,” which means “sandy.” The arenaviruses are divided into the Old World or lymphocytic choriomeningitis virus (LCMV)/LASV complex and the New World or Tacaribe complex (Bowen, Peters, and Nichol, 1997). There is considerable diversity amongst members of the Arenaviridae (FIG. 1), and even within the same virus species (Bowen et al., 2000). In addition to LASV, other arenaviruses that cause severe illness in humans and are classified as BSL-4 and NIAID category A agents include the New World arenaviruses Machupo virus (MACV, Bolivian hemorrhagic fever), Junin virus (JUNV, Argentine hemorrhagic fever), Guanarito virus (GUAV, Venezuelan hemorrhagic fever) and Sabia virus (SABV, Brazilian hemorrhagic fever). Arenaviruses are zoonotic; each virus is associated with a specific species of rodent (Bowen, Peters, and Nichol, 1997). The LCMV/LASV complex viruses are associated with Old World rats and mice (family Muridae, subfamily Murinae). Tacaribe complex viruses are generally associated with New World rats and mice (family Muridae, subfamily Sigmodontinae); however, the reservoir of Tacaribe virus itself appears to be a bat (Bowen, Peters, and Nichol, 1996). The reservoir of LASV is the “multimammate rat” of the genus Mastomys (Monath et al., 1974). Mastomys rats are ubiquitous in sub-Saharan Africa (Demby et al., 2001) and are known to be peridomestic, often living in human homes; however, many questions regarding the taxonomy, geographic distribution and ecobiology of Mastomys species are unanswered. As with the natural hosts of other arenaviruses, Mastomys show no symptoms of LASV infection, but shed the virus in saliva, urine and feces. Eradication of the widely distributed rodent reservoirs of LASV and other arenaviruses is impractical and ecologically undesirable.

Arenaviruses cause chronic infections of rodents across the world with human infections mostly occurring through mucosal exposure or by direct contact of abraded skin with infectious materials. Arenaviruses are easily transmitted to humans from rodents via direct contact with rodent excreta or by contact with or ingestion of excreta-contaminated materials (Bausch et al., 2001; Demby et al., 2001). In the case of Mastomys species, infection may also occur when the animals are caught, prepared as a food source and eaten. Most arenaviruses, including LASV, are readily transmitted between humans, thus making nosocomial infection another matter of great concern. Human-to-human transmission can occur via exposure to blood or body fluids. LASV can also be transmitted to sexual partners of convalescent men via semen up to six weeks post-infection.

Natural history of Lassa fever. Signs and symptoms of Lassa fever, which occur 1-3 weeks after virus exposure, are highly variable, but typically begin with the insidious onset of fever and other nonspecific symptoms such as headache, generalized weakness, and malaise, followed within days by sore throat, retrosternal pain, conjunctival injection, abdominal pain, and diarrhea. LASV infects endothelial cells, resulting in increased capillary permeability, which can produce diminished effective circulating volume (Peters et al., 1989). Severe cases progress to facial and neck swelling, shock and multiorgan system failure. Frank bleeding, usually mucosal (gums, etc.), occurs in less than a third of cases, but confers a poor prognosis. Neurological problems have also been described, including hearing loss, tremors, and encephalitis. Patients who survive begin to defervesce 2-3 weeks after onset of the disease. Temporary or permanent unilateral or bilateral deafness that occurs in a third of Lassa patients during convalescence is not associated with the severity of the acute disease (Cummins et al., 1990; Rybak, 1990; Hartnet et al., 2015; Anderson et al., 2015; Branco et al., October 2011; Grove et al., 2011; Branco et al., 2010; Branco et al., August 2011).

Potential for use of arenaviruses as bioweapons. In addition to high case fatality rates, arenaviruses have many features that enhance their potential as bioweapons. Arenaviruses have relatively stable virions, do not require passage via insect vectors, are spread easily by human-to-human contact, and may be capable of aerosol spread or other simple means of dispersal. The high prevalence of Lassa fever in western Africa coupled with the ease of travel to and from this area and endemic areas for MACV, JUNV, GUAV, SABV and other highly pathogenic arenaviruses permits easy access to these viruses for use as a bioweapon. A cluster of hemorrhagic fever cases in the United States caused by any arenavirus would be a major public health incident. Because febrile illnesses are common, and the use of reliable arenavirus diagnostic tests is not commonplace, an initial cluster of undiagnosed cases would greatly increase the impact of the attack and permit wider dissemination via human-to-human contact. The potential use of LASV and other arenaviruses as a biological weapon directed against civilian or military targets potentiated the commercial development of effective diagnostics, which the VHFC has accomplished, through the marketing of immunodiagnostic tests for the rapid detection of LASV infections (ReLASV Rapid Diagnotic Test [RDT]™, RePanLASV RDT™) and companion ELISA diagnostics for the detection of antigenemia and the immunoglobulin (Ig) M (IgM) and G (IgG) response to infection (www(dot)zalgenlabs(dot)com/products).

Treatment/prevention of arenavirus infections. There are no Food and Drug Administration (FDA)-approved arenavirus vaccines and current anti-arenaviral therapy is limited to an off-label use of the antiviral drug ribavirin that is only partially effective and can cause significant side effects. Ribavirin may be effective in the treatment of Lassa fever only if administered early in the course of illness (Johnson et al., 1987; McCormick et al., 1986). Ribavirin administered to patients with a high virus load (and therefore a high risk for mortality) within the first six days of illness reduced the case-fatality rate from 55% to 5% (McCormick et al., 1986). Several anecdotal reports suggest that this drug can also be effective against other arenaviral hemorrhagic fevers (Barry et al., 1995; Kilgore et al., 1997; Weissenbacher et al., 1986a; Weissenbacher et al., 1986b). The efficacy of prophylactic treatments for Lassa fever is unknown, although it has been suggested that people with high-risk exposures be treated with oral ribavirin. Control of LCMV infection is mediated mainly by cellular immune responses, and significant titers of neutralizing antibodies to LCMV appear usually only after the patients have clinically recovered. However, passive antibody transfer has been shown to confer protection in animal models of LCMV infection (Enria et al., 1984; Frame et al., 1984; Jahrling, 1983; Jahrling and Peters, 1984; Jahrling, Peters, and Stephen, 1984; Weissenbacher et al., 1986a). Thus, antibody-based therapy may provide a safer alternative for treatment of LCMV based on predetermined correlates of protection. Previous studies of passive transfer of serum to treat Argentine hemorrhagic fever (AHF) and Lassa fever provide a strong rationale for the methods disclosed herein. Although passive transfer of serum has proven effective against the New and Old World virus, this approach is not scalable to protect large populations in the case of a hypothetical release of these viruses. Another issue is the safety of transfused serum or plasma, in particular those living in regions where circulating unknown pathogens are of concern. Recombinant, neutralizing, human antibodies have never been tested as potential therapeutics in arenavirus-induced HFs, but these limitations can be overcome. No arenavirus vaccine is currently available, although vaccines against LASV and JUNV are in development. Effective diagnostic assays are absolutely essential for development and field testing arenaviral vaccines.

Antibody-based therapy to combat human viral infections. Viral antigenic variability can pose significant obstacles to the development of effective vaccines to combat human viral infections as illustrated in the cases of HIV and influenza virus. Notably, recent findings have shown that some infected individuals generate broadly neutralizing monoclonal antibodies (BNhMAbs) that target a conserved domain within the stem region of the viral surface envelope (Env) glycoprotein of HIV-1 or and are able to block infection by many phylogenetically distinct isolates. Likewise, a number of BNhMAbs have been shown to target a conserved domain within the proximal membrane stalk domain of influenza virus hemagglutinin (HA) and several BNhMAbs such as MAb F16 and MAb 5A7 proved to be protective when passively administered in mouse models of influenza virus infection. Antibodies typically exhibit desirable pharmacological characteristics including long serum half-lives, high potency, and limited off-target toxicity. Hence, the recent developments in the area of BNhMAbs have raised great interest in exploring their development as viable antiviral therapy. In addition, because BNhMAbs often recognize conserved epitopes within the region of the viral glycoproteins that mediate fusion between viral and cellular membranes, they can also facilitate the identification and structural characterization of highly conserved viral epitopes, knowledge that can be harnessed for the generation of universal vaccines and broad-spectrum antiviral drugs against these viral pathogens. As with HIV-1 and influenza, arenavirus cell entry requires a pH-dependent fusion event that is mediated by the fusogenic domain of GP2. The identification and characterization of LCMV GP-specific BNhMAbs will facilitate the development of a novel antibody-based therapy to treat LASV and LCMV induced disease in humans. In addition, this work may generate valuable information for the design of immunogens to facilitate the development of universal arenavirus vaccines, as well as broad-spectrum anti-arenaviral drugs targeting the conserved structural and functional motifs identified by BNhMAbs.

Need for the invention. The work described herein combines the use of state-of-the-art arenavirus reverse genetics with the access to a unique collection of LASV GP-specific human monoclonal antibodies (hMAbs) that have been shown to cross-react and neutralize different strains of LCMV, including isolates from human cases of LASV and LCMV induced disease, as well as WE strain that causes a LF-like disease in non-human primates. The present disclosure provides an antibody-based therapy to treat human cases of LCMV-induced disease. Unlike vaccines that depend on the host's ability to mount an effective immune response, this novel approach can provide protection in immunosuppressed individuals, including cases of LASV and LCMV infection associated with severe clinical symptoms in individuals undergoing transplantation procedures. Moreover, a detailed characterization of the conserved epitopes within LCMV GPC recognized by these BNhMAbs may help to design immunogens aimed at developing a vaccine able to confer protection against all LASV and LCMV strains that have been linked to disease in humans. In addition, information obtained from the identification and characterization of LASV BNhMAbs will help to identify broad-spectrum anti-LASV and LCMV drugs via targeting conserved epitopes identified by these BNhMAbs. The experimental approach described herein involves the use of unique reagents and assays to identify and characterize LASV and LCMV BNhMAbs and their targets.

There is an ongoing need to address LASV and LCMV infections from natural sources, as well as weaponized versions of these viruses. There also is a need for neutralizing antibodies to LASV and LCMV for diagnostic and analytical uses. The materials (e.g., antibodies and fragments thereof) and methods described herein address these needs.

### SUMMARY

A single-cycle infectious, GFP-expressing, rLCMV has been generated in which the GP is replaced by GFP (rLCMVΔGP/GFP). Genetic complementation with plasmids or stable cell lines expressing arenavirus GPs of interest results in production of the corresponding GP-pseudotyped rLCMVΔGP/GFP that are used to evaluate neutralizing antibody responses to different LCMV strains using a novel GFP-based microneutralization assay. A tri-segmented LCMV platform has been developed within the backone of ARM or Cl-13 LCMV strains that allows expression of an arenavirus GP of choice and an appropriate reporter gene (e.g. fluorescent and luciferase proteins) together for facile identification of LCMV BNhMAbs and monitoring the emergence of BNhMAb LCMV escape mutants. Reverse genetics approaches have been developed that allow generation of rLCM viruses within the backbone of the immunosuppressive Cl-13 LCMV strain expressing GPs of interest that can be used to characterize the therapeutic value in vivo of these BNhMAbs. Highly specific anti-idiotypic antibodies were generated to individually detect and characterize the PK, concentration, and clearance from the circulation of each MAb used in combination therapy to enhance neutralization potency while minimizing the emergence of escape mutants. A panel of anti-idiotype antibodies (anti-ids) to 37.2D specifically detected this BNhMAb when spiked into human serum and did not capture or detect any other arenaviral BNhMAb tested to date, or any other IgG specificity present in human serum on both ELISA and SPR based studies.

Disclosed herein are compositions comprising arenavirus monoclonal antibodies (e.g., fully human monoclonal antibodies), as well as therapeutic, diagnostic, and preventative methods using the antibodies. Preventative methods include preparation of vaccines, as well as factors (e.g. small molecules, peptides) that inhibit Old World arenavirus infectivity, including LASV and LCMV. Diagnostic and therapeutic antibodies including neutralizing antibodies for the prevention and treatment of infection by LASV and other arenaviruses are also disclosed, as well as new tools and methods for the design, production, and use of arenavirus monoclonal antibodies, including expression in engineered bacterial- and mammalian-based systems.

One embodiment of the materials and methods described herein relates to monoclonal antibodies and fragments thereof effective against LASV.

Another embodiment of the materials and methods described herein relates to monoclonal antibodies or fragments thereof effective against LCMV.

Another embodiment of the materials and methods described herein relates to methods of producing forms of monoclonal antibodies effective against LASV and/or LCMV.

Another embodiment of the materials and methods described herein relates to expression vectors comprising polynucleotides encoding forms of the LASV or LCMV GP-specific hMAbs.

An embodiment of the materials and methods described herein relates to diagnostic uses of antibodies or fragments thereof, such as neutralizing antibodies, specific for LASV or LCMV.

Another embodiment of the materials and methods described herein relates to diagnostics comprising the antibodies or fragments thereof specific for LASV or LCMV, including labeled antibodies or fragments thereof of the invention.

Another embodiment of the materials and methods described herein is directed to kits comprising the antibodies of the invention.

The following non-limiting embodiments are provided to illustrate certain aspects and feature of the materials and methods described herein.

Embodiment 1 is an antigen-binding composition comprising a neutralizing antibody or neutralizing antigen-binding antibody fragment thereof specific to glycoprotein 1 (GP1), glycoprotein 2 (GP2), glycoprotein precursor (GPC), or full-length glycoprotein (GP) of Lassa virus (LASV), wherein the antibody or antibody fragment comprises a heavy chain variable region (V_{H}) and a light chain variable region (V_{L}), the V_{H} and V_{L} each comprising complementarity determining regions CDR1, CDR2 and CDR3 selected from the group consisting of:
(a) a V_{H} CDR1 of SEQ ID NO: 65, a V_{H} CDR2 of SEQ ID NO: 66, a V_{H} CDR3 of SEQ ID NO: 67, a V_{L} CDR1 of SEQ ID NO: 113, a V_{L} CDR2 of sequence Gly Ala Ser, and a V_{L} CDR3 of SEQ ID NO: 114 (from MAb 10.4B);
(b) a V_{H} CDR1 of SEQ ID NO: 68, a V_{H} CDR2 of SEQ ID NO: 69, a V_{H} CDR3 of SEQ ID NO: 70, a V_{L} CDR1 of SEQ ID NO: 115, a V_{L} CDR2 of of sequence Lys Ala Ser, and a V_{L} CDR3 of SEQ ID NO: 116 (from MAb 19.7E);
(c) a V_{H} CDR1 of SEQ ID NO: 71, a V_{H} CDR2 of SEQ ID NO: 72, a V_{H} CDR3 of SEQ ID NO: 73, a V_{L} CDR1 of SEQ ID NO: 117, a V_{L} CDR2 of sequence Trp Ala Ser, and a V_{L} CDR3 of SEQ ID NO: 118 (from MAb 2.9D);
(d) a V_{H} CDR1 of SEQ ID NO: 74, a V_{H} CDR2 of SEQ ID NO: 75, a V_{H} CDR3 of SEQ ID NO: 76, a V_{L} CDR1 of SEQ ID NO: 119, a V_{L} CDR2 of sequence Glu Val Lys, and a V_{L} CDR3 of SEQ ID NO: 120 (from MAb 25.6A);
(e) a V_{H} CDR1 of SEQ ID NO: 77, a V_{H} CDR2 of SEQ ID NO: 78, a V_{H} CDR3 of SEQ ID NO: 79, a V_{L} CDR1 of SEQ ID NO: 121, a V_{L} CDR2 of sequence Asp Ala Ser, and a V_{L} CDR3 of SEQ ID NO: 122 (from MAb 36.1F);
(f) a V_{H} CDR1 of SEQ ID NO: 80, a V_{H} CDR2 of SEQ ID NO: 81, a V_{H} CDR3 of SEQ ID NO: 82, a V_{L} CDR1 of SEQ ID NO: 123, a V_{L} CDR2 of sequence Gly Ala Ser, and a V_{L} CDR3 of SEQ ID NO: 124 (from MAb 36.9F);
(g) a V_{H} CDR1 of SEQ ID NO: 83, a V_{H} CDR2 of SEQ ID NO: 84, a V_{H} CDR3 of SEQ ID NO: 85, a V_{L} CDR1 of SEQ ID NO: 125, a V_{L} CDR2 of sequence Gly Ala Ser, and a V_{L} CDR3 of SEQ ID NO: 126 (from MAb 37.2D);
(h) a V_{H} CDR1 of SEQ ID NO: 86, a V_{H} CDR2 of SEQ ID NO: 87, a V_{H} CDR3 of SEQ ID NO: 88, a V_{L} CDR1 of SEQ ID NO: 127, a V_{L} CDR2 of sequence Gly Ala Ser, and a V_{L} CDR3 of SEQ ID NO: 128 (from MAb 37.2G);
(i) a V_{H} CDR1 of SEQ ID NO: 89, a V_{H} CDR2 of SEQ ID NO: 90, a V_{H} CDR3 of SEQ ID NO: 91, a V_{L} CDR1 of SEQ ID NO: 129, a V_{L} CDR2 of sequence Glu Val Arg, and a V_{L} CDR3 of SEQ ID NO: 130 (from MAb 37.7H);
(j) a V_{H} CDR1 of SEQ ID NO: 92, a V_{H} CDR2 of SEQ ID NO: 93, a V_{H} CDR3 of SEQ ID NO: 94, a V_{L} CDR1 of SEQ ID NO: 131, a V_{L} CDR2 of sequence Glu Val Ser, and a V_{L} CDR3 of SEQ ID NO: 132 (from MAb 8.9F);
(k) a V_{H} CDR1 of SEQ ID NO: 95, a V_{H} CDR2 of SEQ ID NO: 96, a V_{H} CDR3 of SEQ ID NO: 97, a V_{L} CDR1 of SEQ ID NO: 133, a V_{L} CDR2 of sequence Gly Ala Ser, and a V_{L} CDR3 of SEQ ID NO: 134 (from MAb NE13);
(l) a V_{H} CDR1 of SEQ ID NO: 98, a V_{H} CDR2 of SEQ ID NO: 99, a V_{H} CDR3 of SEQ ID NO: 100, a V_{L} CDR1 of SEQ ID NO: 135, a V_{L} CDR2 of sequence Gly Ala Ser, and a V_{L} CDR3 of SEQ ID NO: 136 (from MAb 12.1F);
(m) a V_{H} CDR1 of SEQ ID NO: 101, a V_{H} CDR2 of SEQ ID NO: 102, a V_{H} CDR3 of SEQ ID NO: 103, a V_{L} CDR1 of SEQ ID NO: 137, a V_{L} CDR2 of sequence Gln Ala Ser, and a V_{L} CDR3 of SEQ ID NO: 138 (from MAb 9.8A);
(n) a V_{H} CDR1 of SEQ ID NO: 104, a V_{H} CDR2 of SEQ ID NO: 105, a V_{H} CDR3 of SEQ ID NO: 106, a V_{L} CDR1 of SEQ ID NO: 139, a V_{L} CDR2 of sequence Gly Ala Ser, and a V_{L} CDR3 of SEQ ID NO: 140 (from MAb 18.5C);
(o) a V_{H} CDR1 of SEQ ID NO: 107, a V_{H} CDR2 of SEQ ID NO: 108, a V_{H} CDR3 of SEQ ID NO: 109, a V_{L} CDR1 of SEQ ID NO: 141, a V_{L} CDR2 of sequence Gly Ala Tyr, and a V_{L} CDR3 of SEQ ID NO: 142 (from MAb 8.11 G); and
(p) a V_{H} CDR1 of SEQ ID NO: 110, a V_{H} CDR2 of SEQ ID NO: 111, a V_{H} CDR3 of SEQ ID NO: 112, a V_{L} CDR1 of SEQ ID NO: 143, a V_{L} CDR2 of sequence Ala Ala Val, and a V_{L} CDR3 of SEQ ID NO: 144 (from MAb 25.10C).

Embodiment 2 is the composition of Embodiment 1, wherein the composition comprises two or more of said antibodies or antigen-binding antibody fragments.

Embodiment 3 is the composition of any one of Embodiments 1 and 2, wherein the composition comprises:
(1) an antibody or antigen-binding antibody fragment comprising a V_{H} CDR1 of SEQ ID NO: 83, a V_{H} CDR2 of SEQ ID NO: 84, a V_{H} CDR3 of SEQ ID NO: 85, a V_{L} CDR1 of SEQ ID NO: 125, a V_{L} CDR2 of sequence Gly Ala Ser, and a V_{L} CDR3 of SEQ ID NO: 126 (from MAb 37.2D);
(2) an antibody or antigen-binding antibody fragment comprising a V_{H} CDR1 of SEQ ID NO: 92, a V_{H} CDR2 of SEQ ID NO: 93, a V_{H} CDR3 of SEQ ID NO: 94, a V_{L} CDR1 of SEQ ID NO: 131, a V_{L} CDR2 of sequence Glu Val Ser, and a V_{L} CDR3 of SEQ ID NO: 132 (from MAb 8.9F); and
(3) an antibody or antigen-binding antibody fragment comprising a V_{H} CDR1 of SEQ ID NO: 98, a V_{H} CDR2 of SEQ ID NO: 99, a V_{H} CDR3 of SEQ ID NO: 100, a V_{L} CDR1 of SEQ ID NO: 135, a V_{L} CDR2 of sequence Gly Ala Ser, and a V_{L} CDR3 of SEQ ID NO: 136 (from MAb 12.1F).

Embodiment 4 is the composition of any one of Embodiments 1 to 3, wherein the antibody is selected from the group consisting of a monoclonal antibody, and a recombinantly produced antibody.

Embodiment 5 is the composition of any one of Embodiments 1 to 4, wherein the antibody comprises a human monoclonal antibody.

Embodiment 6 is the composition of any one of Embodiments 1 to 3, wherein the antigen-binding antibody fragment is selected from the group consisting of a Fab, a Fab′, and a F(ab′)₂ fragment.

Embodiment 7 is a nucleic acid (e.g., a cDNA) having a sequence that encodes for a V_{H} of the antibody or the antibody fragment of a composition of Embodiment 1.

Embodiment 8 is the nucleic acid of Embodiment 7, wherein the nucleic acid includes a nucleic acid sequence selected from the group consisting of SEQ ID NO: 1 through SEQ ID NO: 16.

Embodiment 9 is a nucleic acid 9e.g., a cDNA) having a sequence that encodes for a V_{L} of the antibody or the antibody fragment of Embodiment 1.

Embodiment 10 is the nucleic acid of Embodiment 9, wherein the nucleic acid includes a nucleic acid sequence selected from the group consisting of SEQ ID NO: 17 through SEQ ID NO: 32.

Embodiment 11 is an expression vector that contains the nucleic acid sequence of any one of Embodiments 7 to 10.

Embodiment 12 is an antigen-binding composition comprising a neutralizing antibody or neutralizing antigen-binding antibody fragment thereof specific to glycoprotein 1 (GP1), glycoprotein 2 (GP2), glycoprotein precursor (GPC), or full-length glycoprotein (GP) of Lassa virus (LASV), wherein the antibody or antibody fragment comprises a heavy chain variable region (V_{H}) and a light chain variable region (V_{L}) selected from the group consisting of:
(a) a V_{H} of SEQ ID NO: 33 and a V_{L} of SEQ ID NO: 49 (from MAb 10.4B);
(b) a V_{H} of SEQ ID NO: 34 and a V_{L} of SEQ ID NO: 50 (from MAb 19.7E);
(c) a V_{H} of SEQ ID NO: 35 and a V_{L} of SEQ ID NO: 51 (from MAb 2.9D);
(d) a V_{H} of SEQ ID NO: 36 and a V_{L} of SEQ ID NO: 52 (from MAb 25.6A);
(e) a V_{H} of SEQ ID NO: 37 and a V_{L} of SEQ ID NO: 53 (from MAb 36.1F);
(f) a V_{H} of SEQ ID NO: 38 and a V_{L} of SEQ ID NO: 54 (from MAb 36.9F);
(g) a V_{H} of SEQ ID NO: 39 and a V_{L} of SEQ ID NO: 55 (from MAb 37.2D);
(h) a V_{H} of SEQ ID NO: 40 and a V_{L} of SEQ ID NO: 56 (from MAb 37.2G);
(i) a V_{H} of SEQ ID NO: 41 and a V_{L} of SEQ ID NO: 57 (from MAb 37.7F);
(j) a V_{H} of SEQ ID NO: 42 and a V_{L} of SEQ ID NO: 58 (from MAb 8.9F);
(k) a V_{H} of SEQ ID NO: 43 and a V_{L} of SEQ ID NO: 59 (from MAb NE13);
(l) a V_{H} of SEQ ID NO: 44 and a V_{L} of SEQ ID NO: 60 (from MAb 12.1F);
(m) a V_{H} of SEQ ID NO: 45 and a V_{L} of SEQ ID NO: 61 (from MAb 9.8A);
(n) a V_{H} of SEQ ID NO: 46 and a V_{L} of SEQ ID NO: 62 (from MAb 18.5C);
(o) a V_{H} of SEQ ID NO: 47 and a V_{L} of SEQ ID NO: 63 (from MAb 8.11G); and
(p) a V_{H} of SEQ ID NO: 48 and a V_{L} of SEQ ID NO: 64 (from MAb 25.10C).

Embodiment 13 is the composition of Embodiment 12, wherein the composition comprises two or more of said antibodies or antigen-binding antibody fragments. Embodiment 14 is the composition of any one of Embodiments 12 and 13, wherein the composition comprises:
(1) an antibody or antigen-binding antibody fragment comprising a V_{H} of SEQ ID NO: 39 and a V_{L} of SEQ ID NO: 55 (from MAb 37.2D);
(2) an antibody or antigen-binding antibody fragment comprising a V_{H} of SEQ ID NO: 42 and a V_{L} of SEQ ID NO: 58 (from MAb 8.9F); and
(3) an antibody or antigen-binding antibody fragment comprising a V_{H} of SEQ ID NO: 44 and a V_{L} of SEQ ID NO: 60 (from MAb 12.1F).

Embodiment 15 is the composition of any one of Embodiments 12 to 14, wherein the the antibody is selected from the group consisting of a monoclonal antibody, and a recombinantly produced antibody.

Embodiment 16 is the composition of any one of Embodiments 12 to 15, wherein the antibody comprises a human monoclonal antibody.

Embodiment 17 is the composition of any one of Embodiments 12 to 14, wherein the antigen-binding antibody fragment is selected from the group consisting of a Fab, a Fab′, and a F(ab′)₂ fragment.

Embodiment 18 is a nucleic acid (e.g., a cDNA) having a sequence that encodes for a V_{H} of the antibody or the antibody fragment of Embodiment 12.

Embodiment 19 is a nucleic acid (e.g., a cDNA) having a sequence that encodes for a V_{L} of the antibody or the antibody fragment of Embodiment 12.

Embodiment 20 is an expression vector that contains the nucleic acid sequence of any one of Embodiments 18 to 19.

Embodiment 21 is a vaccine for preventing or treating infection of a patient by Lassa virus or other arenaviridae comprising the antibody or antibody fragment of any one of Embodiments 1 to 6 and 12 to 17.

Embodiment 22 is the vaccine of Embodiment 21, which is cross-protective against infection by other arenaviridae.

Embodiment 23 is the vaccine of any one of Embodiments 21 to 22, which is cross-protective against infection by a lymphocytic choriomeningitis virus.

Embodiment 24 is a pharmaceutical composition for treating or preventing infection by a Lassa virus or other arenaviridae comprising the antibody or antibody fragment of any one of Embodiments 1 to 6 and 12 to 17 and a pharmaceutically acceptable carrier.

Embodiment 25 is the antibody or antibody fragment of any one of Embodiments 1 to 6 and 12 to 17 for use in treating or preventing infection by a Lassa virus or other arenaviridae.

Embodiment 26 is the antibody or antibody fragment of any one of Embodiments 1 to 6 and 12 to 17 for use in treating or preventing a lymphocytic choriomeningitis virus infection.

Embodiment 27 is use of the antibody or antibody fragment of any one of Embodiments 1 to 6 and 12 to 17 for treating or preventing infection by a Lassa virus or other arenaviridae.

Embodiment 28 is use of the antibody or antibody fragment of any one of Embodiments 1 to 6 and 12 to 17 for treating or preventing a lymphocytic choriomeningitis virus infection.

Embodiment 29 is use of the antibody or antibody fragment of any one of Embodiments 1 to 6 and 12 to 17 for the preparation of a medicament for treating or preventing infection by a Lassa virus or other arenaviridae.

Embodiment 30 is use of the antibody or antibody fragment of any one of Embodiments 1 to 6 and 12 to 17 for the preparation of a medicament for treating or preventing a lymphocytic choriomeningitis virus infection.

Embodiment 31 is diagnostic kit for detecting infection of a subject by Lassa virus or other arenaviridae comprising at least one antibody or antibody fragment of any one of Embodiments 1 to 6 and 12 to 17 bound to a detectable labelling group.

Embodiment 32 is an antibody or antibody fragment of any one of Embodiments 1 to 6 and 12 to 17 bound to a detectable labelling group.

Embodiment 33 is a method of detecting infection by a Lassa virus or other arenaviridae comprising contacting a biological sample from a subject with at least one antibody or antibody fragment of any one of Embodiments 1 to 6 and 12 to 17 bound to a detectable labelling group; and detecting a complex between the antibody or antibody fragment and a Lassa virus or other arenaviridae present in the sample.

Embodiment 34 is a method of treating or preventing infection by a Lassa virus or other arenaviridae in a subject comprising administering the antibody or antibody fragment of any one of Embodiments 1 to 6 and 12 to 17 to the subject.

Embodiment 35 is a method of treating or preventing a lymphocytic choriomeningitis virus infection in a subject comprising administering the antibody or antibody fragment of any one of Embodiments 1 to 6 and 12 to 17 to the subject.

Other embodiments and advantages of the materials and methods described herein are set forth in part in the description, which follows, and in part, may be understood by a person of ordinary skill in the art from this description, or from the practice or use of the materials and methods described herein.

### DESCRIPTION OF THE FIGURES

FIG. 1 depicts (A) Schematic representation of LASV GP; (B) Arenavirus GP complex; and (C) Recognition of different LASV GP species by LASV hMAbs. (A) LASV GP is synthesized as the precursor protein GPC. Signal peptidase (Spase) cleaves the small stable signal peptide (SSP) that remains associated with GP1 and GP2 to form the GP complex (FIG. 1, Panel B). The cellular protease SK1/S1P cleaves GPC into GP1 and GP2. Construct rGPe corresponds to a recombinant LASV GPC ectodomain lacking GP2 and with a non-cleavable linker replacing the SK1/S1P cleavage recognition site. Constructs expressing recombinant SSP-GP1 (rGP1) and SSP fused to GP2 (rGP2) were also generated. (B) GP-1 forms the globular head subunit that interact with the cellular receptor whereas GP2 mediates the fusion of the viral envelop with the cell membrane. SSP remains associated with both GP1 and GP2 and plays critical roles in the biology of the GP complex. (C) 293T cells were transfected with pCAGGS expressing plasmids encoding LASV rGP1, rGP2 and GPC and the reactivity of LASV hMAbs evaluated at 48 h post-transfection by immunofluorescence. The distribution of LASV GP-specific hMAbs by subunit specificity, neutralizing activity and reactivity to linear epitopes is indicated.

FIG. 2 depicts in vitro neutralization of LCMV ARM with the 15 LASV GP-specific neutralizing hMAbs: LASV Josiah (squares) and LCMV ARM (triangles) GP-pseudotyped rLCMVΔGP/GFP viruses were incubated for 90 min at 37° C. with a 2-fold dilution of the indicated LASV GP-specific BNhMAb before infecting LCMV GP-expressing Vero cells (96 plate format, triplicates). Virus neutralization was determined under a fluorescent microscope and quantified using a GFP microplate reader at 72 hours post-infection. Results are presented as percent inhibition after normalizing to respective viral infections in the absence of hMAbs. Virus infection in the absence of hMAbs was used as internal control. Mean values and standard deviation are shown. Standard error was calculated based on 2-6 replicates.

FIG. 3 depicts in vivo neutralization of the 6 LCMV neutralizing antibodies (12.1F, 9.8A, 37.2D, 36.9F, 37.2G, and 18.5C) using the non-crossreactive antibodies 19.7E and 8.9F as internal controls. Mice were infected with rCl-13 (2×106 pfu; i.v.) and treated with the indicated hMAb (20 mg/kg; i.p.), as well as an isotype hMAb control (20 mg/kg, i.p.) or vehicle. At days 4 and 21, post inoculation (p.i.) viremia (i.e., the presence of viruses in the blood) was determined. Results correspond to the average and standard deviation (SD) of four mice/group; LoD=limit of detection.

FIG. 4 illustrates Viremia data from treated and control GP plasma on days 7 and 14 PI. Viremia levels for day 7 treatment groups 37.7H, 12.1F, and 25.6A as well as day 14 12.1F, 37.2D, 19.7E, and 10.4B were below the limit of detection (LOD). Error bars represent standard deviation from mean values. *denotes P_0.05. **denotes P_0.001. ***denotes P<0.0001.

FIG. 5 depicts clinical scores of HuMAb treated and untreated guinea pigs. HuMAbs 8.9F and 12.1 treated GP showed no variation in clinical score from baseline (data not shown). Error bars (thin lines) represent standard deviation from mean values.

FIG. 6 illustrates the effect of antibodies on rVSV-LASV GP infection and fusion. Antibody-mediated neutralization of (A) rVSV-LASV GP or (B) rVSV-VSV-G. The antibody 9.7A is non-neutralizing and in the same competition group as 37.7H (GPC-B); 13.4E binds to a linear epitope in the T-loop of GP2; 12.1F binds to the GP1 subunit of LASV. Error bars indicate the standard deviation of at least six (two biological replicates, each having three or more technical replicates). (C) Antibody-mediated inhibition of rVSVLASV GP fusion at the cell surface. Error bars indicate the standard error of the mean of six (except 37.7H, where N=9). (D) Fab 37.7H reduces binding of a LAMP1-Fc fusion protein to LASV GPCysR4. Error bars indicate the standard deviation of six and three technical replicates.

FIG. 7 provides a sequence alignment prepared using CLUSTAL OMEGA™ (1.2.4) multiple sequence alignment (from EMBL-EBI, a part of the European Molecular Biology Laboratory) for the heavy chain variable region amino acid sequences, with CDRs highlighted in bold typeface: CDR1 (marked with +), CDR 2 (marked with \hat{ }), and CDR3 (marked with #). The sequences shown include 10.4B (SEQ ID NO: 33), 19.7E (SEQ ID NO: 34), 2.9D (SEQ ID NO: 35), 25.6A (SEQ ID NO: 36), 36.1F (SEQ ID NO: 37), 36.9F (SEQ ID NO: 38), 37.2D (SEQ ID NO: 39), 37.2G (SEQ ID NO: 40), 37.7H (SEQ ID NO: 41), 8.9F (SEQ ID NO: 42), NE13 (SEQ ID NO: 43), 12.1F (SEQ ID NO: 44), 9.8A (SEQ ID NO: 45), 18.5C (SEQ ID NO: 46), 8.11G (SEQ ID NO: 47), and 25.10C (SEQ ID NO: 48).

FIG. 8 provides a sequence alignment prepared using using CLUSTAL OMEGA™ (1.2.4) multiple sequence alignment for the light chain variable region amino acid sequences, with CDRs highlighted in bold typeface: CDR1 (marked with +), CDR 2 (marked with \hat{ }), and CDR3 (marked with #). The sequences shown include 10.4B (SEQ ID NO: 49), 19.7E (SEQ ID NO: 50), 2.9D (SEQ ID NO: 51), 25.6A (SEQ ID NO: 52), 36.1F (SEQ ID NO: 53), 36.9F (SEQ ID NO: 54), 37.2D (SEQ ID NO: 55), 37.2G (SEQ ID NO: 56), 37.7H (SEQ ID NO: 57), 8.9F (SEQ ID NO: 58), NE13 (SEQ ID NO: 59), 12.1F (SEQ ID NO: 60), 9.8A (SEQ ID NO: 61), 18.5C (SEQ ID NO: 62), 8.11G (SEQ ID NO: 63), and 25.10C (SEQ ID NO: 64).

### DETAILED DESCRIPTION

### General Techniques

The practice of the materials and methods described herein will employ, unless otherwise indicated, conventional techniques of molecular biology (including recombinant techniques), microbiology, cell biology, biochemistry, and immunology, which are all within the normal skill of the art. Such techniques are fully explained in the literature, such as, for example, Molecular Cloning: A Laboratory Manual, second edition (Sambrook, et al., 1989) Cold Spring Harbor Press; Methods in Molecular Biology, Humana Press; Cell Biology: A Laboratory Notebook (I. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Cabs, eds., 1987); Current Protocols in Molecular Biology (F. M. Ausubel, et aL, eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

As used herein, the singular form “a”, “an”, and “the” includes plural references unless indicated otherwise. For example, “a” monoclonal antibody includes one or more monoclonal antibodies.

Generally, monoclonal antibodies specific for LASV, monoclonal antibodies specific for LCMV, the polynucleotides encoding the antibodies, and methods for using these antibodies in prevention, diagnosis, detection, and treatment are described herein. Specifically, human monoclonal antibodies specific for LASV, human monoclonal antibodies specific for LCMV, and combinations thereof for development and production of diagnostics, vaccines, therapeutics, and screening tools are provided. Generally, B cell clones producing specific IgG to GP of any Lassa virus isolate or strain may be utilized to derive the antibodies described herein.

### Polynucleotides

The term polynucleotide is used broadly and refers to polymeric nucleotides of any length (e.g., oligonucleotides, genes, small inhibiting RNA, fragments of polynucleotides encoding a protein, etc.). By way of example and not limitation, the polynucleotides of the invention may comprise a sequence encoding all or part of the ectodomain and part of the transmembrane domain. The polynucleotide of the invention may be, for example, linear, circular, supercoiled, single-stranded, double-stranded, branched, partially double-stranded or partially single-stranded. The nucleotides comprised within the polynucleotide may be naturally occurring nucleotides or modified nucleotides.

Functional equivalents of these polynucleotides are also intended to be encompassed by this invention. By way of example and not limitation, functionally equivalent polynucleotides are those that possess one or more of the following characteristics: the ability to generate antibodies (including, but not limited to, viral neutralizing antibodies) capable of recognizing LASV GP or the ability to generate antibodies specific to LASV GP that show neutralizing activity against LASV lineages I-IV, and proposed new lineages (e.g. lineage V from Mali, lineage VI from Togo and Benin.

Polynucleotide sequences that are functionally equivalent may also be identified by methods known in the art. A variety of sequence alignment software programs are available to facilitate determination of homology or equivalence. Non-limiting examples of these programs are BLAST family programs including BLASTN, BLASTP, BLASTX, TBLASTN, and TBLASTX (BLAST is available from the National Institutes of Health website), FASTA™, COMPARE™, DOTPLOT™, BESTFIT™ GAP™ FRAMEALIGN™, CLUSTALW™, and PILEUP™. Other similar analysis and alignment programs can be purchased from various providers such as DNA Star's MEGALIGN™, or the alignment programs in GENEJOCKEY™. Alternatively, sequence analysis and alignment programs can be accessed through the world wide web at sites such as the CMS Molecular Biology Resource at San Diego Supercomuter Center (SDSC) website; and the Swiss Institute of Bioinformatics SIB Bioinformatics Resource Portal website ExPASy Proteomics Server. Any sequence database that contains DNA or protein sequences corresponding to a gene or a segment thereof can be used for sequence analysis. Commonly employed databases include but are not limited to GenBank, EMBL, DDBJ, PDB, SWISS-PROT, EST, STS, GSS, and HTGS.

Parameters for determining the extent of homology set forth by one or more of the aforementioned alignment programs are well established in the art. They include but are not limited to p value, percent sequence identity and the percent sequence similarity. P value is the probability that the alignment is produced by chance. For a single alignment, the p value can be calculated according to Karlin et al. (1990) Proc. Natl. Acad. Sci. (USA) 87: 2246. For multiple alignments, the p value can be calculated using a heuristic approach such as the one programmed in BLAST. Percent sequence identify is defined by the ratio of the number of nucleotide or amino acid matches between the query sequence and the known sequence when the two are optimally aligned. The percent sequence similarity is calculated in the same way as percent identity except one scores amino acids that are different but similar as positive when calculating the percent similarity. Thus, conservative changes that occur frequently without altering function, such as a change from one basic amino acid to another or a change from one hydrophobic amino acid to another are scored as if they were identical.

The term “analog” includes any polypeptide having an amino acid residue sequence substantially identical to a polypeptide of the invention in which one or more residues have been conservatively substituted with a functionally similar residue and which displays the functional aspects of the polypeptides as described herein. Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; and the substitution of one acidic residue, such as aspartic acid or glutamic acid or another.

The phrase “conservative substitution” also includes the use of a chemically derivatized residue in place of a non-derivatized residue. “Chemical derivative” refers to a subject polypeptide having one or more amino acid residues chemically derivatized by reaction of a functional side group. Examples of such derivatized amino acids include for example, those amino acids in which free amino groups have been derivatized to form amine hydrochlorides, p-toluene sulfonyl groups, carbobenzoxy groups, t-butyloxycarbonyl groups, chloroacetyl groups or formyl groups. Also, the free carboxyl groups of amino acids may be derivatized to form salts, methyl and ethyl esters or other types of esters or hydrazides. Also, the free hydroxyl groups of certain amino acids may be derivatized to form 0-acyl or 0-alkyl derivatives. Also, the imidazole nitrogen of histidine may be derivatized to form N-imbenzylhistidine. Also included as chemical derivatives are those proteins or peptides which contain one or more naturally occurring amino acid derivatives of the twenty standard amino acids. For example, 4-hydroxyproline may be substituted for proline, 5-hydroxylysine may be substituted for lysine, 3-methylhistidine may be substituted for histidine, homoserine may be substituted for serine, and ornithine may be substituted for lysine. Polypeptides of the present invention also include any polypeptide having one or more additions and/or deletions of residues relative to the sequence of any one of the polypeptides whose sequence is described herein.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75 contiguous positions, or 40 to about 50 contiguous positions, in which a sequence may be compared to a reference sequence of the same number of contiguous positions after the two sequences are optimally aligned.

Optimal alignment of sequences for comparison may be conducted using the MEGALIGN™ program in the LASERGENE™ suite of bioinformatics software (DNASTAR, Inc., Madison, WI), using default parameters. This program embodies several alignment schemes described in the following references: Dayhoff, M. O. (1978) “A model of evolutionary change in proteins—Matrices for detecting distant relationships” in Dayhoff, M. O. (ed.) Atlas of Protein Sequence and Structure, National Biomedical Research Foundation, Washington DC Vol. 5, Suppl. 3, pp. 345-358 (1978); Hem J., “Unified Approach to Alignment and Phylogenes” pp. 626-645 Methods in Enzymology vol. 183, Academic Press, Inc., San Diego, CA (1990); Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, CA; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

### Expression Vectors

Expression vectors comprising at least one polynucleotide encoding an antibody or antibody fragment protein also are described herein. Expression vectors are well known in the art and include, but are not limited to viral vectors or plasmids. Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127), alphavirus-based vectors (e.g., Sindbis virus vectors, Semliki forest virus), Ross River virus, adeno-associated virus (AAV) vectors (see, e.g., PCT Publication Nos. WO 94/12649, WO 93/03769; WO 93/19191; WO 94/28938; WO 95/11984 and WO 95/00655), vaccinia virus (e.g., Modified Vaccinia virus Ankara (MVA) or fowlpox), Baculovirus recombinant system and herpes virus.

Nonviral vectors, such as plasmids, are also well known in the art and include, but are not limited to, yeast- and bacteria-based plasmids.

Methods of introducing the vectors into a host cell and isolating and purifying the expressed protein are also well known in the art (e.g., Molecular Cloning: A Laboratory Manual, second edition, Sambrook, et al., 1989, Cold Spring Harbor Press). Examples of host cells include, but are not limited to, mammalian cells such as NS0 and CHO cells.

By way of example, vectors comprising the polynucleotides described herein may further comprise a tag polynucleotide sequence to facilitate protein isolation and/or purification. Examples of tags include but are not limited to the myc-epitope, S-tag, his-tag, HSV epitope, V5-epitope, FLAG and CBP (calmodulin binding protein). Such tags are commercially available or readily made by methods known to the art.

The vector may further comprise a polynucleotide sequence encoding a linker sequence. Generally, the linking sequence is positioned in the vector between the antibody polynucleotide sequence and the polynucleotide tag sequence. Linking sequences can encode random amino acids or can contain functional sites. Examples of linking sequences containing functional sites include but are not limited to, sequences containing the Factor Xa cleavage site, the thrombin cleavage site, or the enterokinase cleavage site.

By way of example, and not limitation, an antibody specific for LASV may be generated as described herein using mammalian expression vectors in mammalian cell culture systems or bacterial expression vectors in bacterial culture systems. By way of example, and not limitation, an antibody specific for LCMV may be generated as described herein using mammalian expression vectors in mammalian cell culture systems or bacterial expression vectors in bacterial culture systems.

### Antibodies

Examples of antibodies disclosed herein, include, but are not limited to, antibodies specific for LASV or LCMV, antibodies that cross react with native Lassa virus antigens and/or native lymphocytic choriomeningitis virus antigens, and neutralizing antibodies. By way of example, a characteristic of a neutralizing antibody includes the ability to block or prevent infection of a host cell. The antibodies may be characterized using methods well known in the art.

The antibodies useful in the compositions and methods described herein can encompass monoclonal antibodies, polyclonal antibodies, antibody fragments (e.g., Fab, Fab′, F(ab′)2, Fv, Fc, etc.), chimeric antibodies, bi-specific antibodies, heteroconjugate antibodies, single-chain fragments (e.g. ScFv), mutants thereof, fusion proteins comprising an antibody portion, humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or of any other origin (including chimeric or humanized antibodies).

Methods of preparing monoclonal and polyclonal antibodies are well known in the art. Polyclonal antibodies can be raised in a mammal, for example, by one or more injections of an immunizing agent and, if desired an adjuvant. Examples of adjuvants include, but are not limited to, keyhole limpet hemocyanin (KLH), serum albumin, bovine thryoglobulin, soybean trypsin inhibitor, complete Freund adjuvant (CFA), and MPL-TDM adjuvant. The immunization protocol can be determined by one of skill in the art.

The antibodies may alternatively be monoclonal antibodies. Monoclonal antibodies may be produced using hybridoma methods (see, e.g., Kohler, B. and Milstein, C. (1975) Nature 256:495-497 or as modified by Buck, D. W., et al., In Vitro, 18:377-381(1982).

If desired, the antibody of interest may be sequenced and the polynucleotide sequence may then be cloned into a vector for expression or propagation. The sequence encoding the antibody of interest may be maintained in the vector in a host cell, and the host cell can then be expanded and frozen for future use. In an alternative embodiment, the polynucleotide sequence may be used for genetic manipulation to “humanize” the antibody or to improve the affinity, or other characteristics of the antibody (e.g., genetically manipulate the antibody sequence to obtain greater affinity to LASV and/or LCMV glycoprotein and/or greater efficacy in inhibiting the fusion of LASV and/or LCMV to the host cell).

The antibodies may also be humanized by methods known in the art (See, for example, U.S. Pat. Nos. 4,816,567; 5,807,715; 5,866,692; 6,331,415; 5,530,101; 5,693,761; 5,693,762; 5,585,089; and 6,180,370). In yet another alternative, human antibodies may be obtained by using mice that have been engineered to express specific human immunoglobulin proteins.

In another alternative embodiment, antibodies may be made recombinantly and expressed using any method known in the art. By way of example, antibodies may be made recombinantly by phage display technology. See, for example, U.S. Pat. Nos. 5,565,332; 5,580,717; 5,733,743; and 6,265,150; and Winter et at., Annu. Rev. Immunol. 12:433-455 (1994). Alternatively, phage display technology (McCafferty et al., Nature 348:552-553 (1990)) can be used to produce human antibodies and antibody fragments in vitro. Phage display can be performed in a variety of formats; for review, see Johnson, Kevin S. and Chiswell, David J., Current Opinion in Structural Biology 3:564-571 (1993). By way of example, LASV and/or LCMV glycoprotein as described herein may be used as an antigen for the purposes of isolating recombinant antibodies by these techniques.

Antibodies may be made recombinantly by first isolating the antibodies and antibody producing cells from host animals, obtaining the gene sequence, and using the gene sequence to express the antibody recombinantly in host cells (e.g., CHO cells). Another method that may be employed is to express the antibody sequence in plants (e.g., tobacco) or transgenic milk. Methods for expressing antibodies recombinantly in plants or milk have been disclosed. See, for example, Peeters, et al. Vaccine 19:2756 (2001); Lonberg, N. and D. Huszar Int. Rev. Immunol 13:65 (1995); and Pollock, et al., J. Immunol. Methods 231:147 (1999). Methods for making derivatives of antibodies (e.g. humanized and single-chain antibodies, etc.) are known in the art.

The antibodies described herein can be bound to a carrier by conventional methods for use in, for example, isolating or purifying LASV and/or LCMV glycoprotein or detecting LASV and/or LCMV glycoproteins, antigens, or particles in a biological sample or specimen. Alternatively, by way of example, the neutralizing antibodies of the invention may be administered as a therapeutic treatment to a subject infected with or suspected of being infected with LASV or LCMV. A “subject,” includes but is not limited to humans, simians, farm animals, sport animals, and pets. Veterinary uses are also encompassed by methods described herein. For diagnostic purposes, the antibodies can be labeled, e.g., bound to a detectable labelling group such as a fluorescent dye (e.g., a ALEXA FLUOR® dye), a quantum dot label (e.g., a QDOT® label), R-phycoerythrin, streptavidin, biotin, an enzyme (e.g., Glucose Oxidase, Horseradish Peroxidase or Alkaline Phosphatase), a radioiosotope (e.g., iodine-125, indium-111), and the like. Such labelling techniques are well known in the antibody art.

### Antibody DNA Sequences

Sixteen neutralizing antibodies against LASV were identified, which are designated herein as 10.4B, 19.7E, 2.9D, 25.6A, 36.1F, 36.9F, 37.2D, 37.2G, 37.7H, 8.9F, NE13, 12.1F, 9.8A, 18.5C, 8.11G, and 25.10C. Nucleotide sequences (cDNA) encoding portions of heavy chain (HC) and light chain (LC) of each antibody are shown below. The illustrated nucleotide sequences encode portions of the HC and LC encompassing the variable regions thereof, i.e., the V_{H} and V_{L} regions, respectively, along with portions of vector sequences.

| (10.4B V_{H}) | |
|---|---|
| SEQ ID NO: 1 | |
| tgcgcgttac ngatccaagc tgtgaccggc gcctacctga gatcaccggt gctagcacca | 60 |
| tggagacaga cacactcctg ctatgggtac tgctgctctg ggttccaggt tccactggtg | 120 |
| accaggtgca gctggtacag tctgggggag gcgtggtcca gcctgggagg tccctgagag | 180 |
| tctcctgtgt tacgtctgga ttcaatttca gagcctacgg catgcactgg gtccgccaga | 240 |
| ttccaggcaa gggactggag tgggtggcag atatttggtc tgccgagact aatagacact | 300 |
| atgcagattc cgtgaagggc cgattcacca tctccagaga caactccaag agcacactgt | 360 |
| atctgcaaat gaacagcctg agagccgagg acacgggcgt atatttctgt gccaaagcgc | 420 |
| gaccaggcta tgattatgtc gttgacttat ggggccaggg aacgctggtc atcgtctcct | 480 |
| cagcttccac caagggccca tcggtcttcc ccctggcgcc ctgctccagg agcacctctg | 540 |
| ggggcacagc ggccctgggc tgcctggtca aggactactt ccccgaaccg gtgacggtgt | 600 |
| cgtggaactc aggcgccctg accagcggcg tgcacacctt cccggctgtc ctacagtcct | 660 |
| caggactcta | 670 |
| (19.7E V_{H}) | |
| SEQ ID NO: 2 | |
| atccagctgt gaccggcgcc tacctgagat caccggtgct agcaccatgg agacagacac | 60 |
| actcctgcta tgggtactgc tgctctgggt tccaggttcc actggtgacg aggtgcagct | 120 |
| ggtggagtct gggggaggct tagttcggcc tggggggtcc ctgagactct cctgtgcagc | 180 |
| ctctggattc tccttcagta gctactcgat gcactgggtc cgccatgttc ctgggaaggg | 240 |
| gctggtgtgg gtctcatata ttaatagtga tgggagtact aaaatctacg cggactccgt | 300 |
| gaagggccga ttctccatct ccagagacaa tgccaagaac aagctctatc tgcaaatgga | 360 |
| cagtttgaga gtcgaggaca cggctgtata ttcgtgtgta aggcttgtac attacgactg | 420 |
| gtccccattc gtgtggggcc agggaaccct ggtcaccgtc tcctcagcct ccaccaaggg | 480 |
| cccatcggtc ttccccctgg caccctcctc caagagcacc tctgggggca cagcggccct | 540 |
| gggctgcctg gtcaaggact acttccccga accggtgacg gtgtcgtgga actcaggcgc | 600 |
| cctgaccagc ggcgtgcaca ccttcccggc tgtcctacag tcctcaggac tctactccct | 660 |
| cagcagcgtg gtgaccgtgc cctccagcag cttgggcacc cagacctaca tctgcaacgt | 720 |
| gaatcacaag cccagcaaca ccaaggtgga caagaaagtt gagccccaat cttgtgacaa | 780 |
| aactcacaca tgcccaccgt gcccagcacc tgaactcct | 819 |
| (2.9D V_{H}) | |
| SEQ ID NO: 3 | |
| gtcactgcac ctcggttcta tcgattggct agcaccatgg agacagacac actcctgcta | 60 |
| tgggtactgc tgctctgggt tccaggttcc actggtgacg aggtgcagct ggtggagtct | 120 |
| gggggaggcc tggtcaagcc tggggggtcc cttagactct cctgtgcagc ctctggattc | 180 |
| accttcacta gatttacttt gacctgggtc cgccaggctc cagggaaggg gctggagtgg | 240 |
| gtctcatcca ttagtagtgg gagtagtgac ataaactacg cagactcagt gaagggccga | 300 |
| ttcaccatat ccagagacaa cgccaggaac tccctgttcc tgcaaatgag cagcctgaga | 360 |
| gtcgacgaca cggctgtgta ttactgtgcg aaagatcccc ggtcggggat ctctggtcgc | 420 |
| tacgggatgg acgtctgggg ccaagggacc acggtcatcg tctcctcagc ttccaccaag | 480 |
| ggcccatcgg tcttccccct ggcgccctgc tccaggagca cctctggggg cacagcggcc | 540 |
| ctgggctgcc tggtcaagga ctacttcccc gaaccggtga cggtgtcgtg gaactcaggc | 600 |
| gccctgacca gcggcgtgca caccttcccg gctgtcctac agtcctcagg actctactcc | 660 |
| ctcagcagcg tggtgaccgt gccctccagc agcttgggca cccagaccta catctgcaac | 720 |
| gtgaatcaca agcccagcaa caccaaggtg gacaagagag ttgagcccaa atcttgtgac | 780 |
| aaaactcaca catgcccacc gtgcccagca cctgaactcc tggggggacc gtcagtcttc | 840 |
| ctcttccccc caaaacccaa ggacaccctc atgatctccc ggacccctga ggtcacatgc | 900 |
| gtggtggtgg acgtgagcca | 920 |
| (25.6A V_{H}) | |
| SEQ ID NO: 4 | |
| acctcggttc ttcgattggc tagcaccatg gagacagaca cactcctgct atgggtactg | 60 |
| ctgctctggg ttccaggttc cactggtgac caggtgcagc tgcaggagtc aggaggaggc | 120 |
| ctggtcaagg ctggggggtc cctgagactc tcctgtgcag cctctggatt catgttcgag | 180 |
| agatatagcc ttcactgggt ccgtcagact ccaggcaagg ggctggagtg ggtctcatcc | 240 |
| attagtagtc ttagtggcag tcacataaac tacgcagact cagtgaaggg ccgattcacc | 300 |
| atctccagag acaacgccaa gaattcactg tctctgcaaa tgaacagcct gagagtcgaa | 360 |
| gacacggcta tatattattg tgcgagagat cgacgttcgg ggagttcccc cgtccccttg | 420 |
| gacgtctggg gccaagggac cacggtcacc gtctcctctg cctccaccaa gggcccatcg | 480 |
| gtcttccccc tggcaccctc ctccaagagc acctctgggg gcacagcggc cctgggctgc | 540 |
| (36.1F V_{H}) | |
| SEQ ID NO: 5 | |
| gtcactgccc tcggttctat cgattggcta gcaccatgga gacagacaca ctcctgctat | 60 |
| gggtactgct gctctgggtt ccaggttcca ctggtgacca ggtgcagctg caggagtcgg | 120 |
| gcgcgggact ggtgaagcct tcggagaccc tgtccctcac ctgcgctgtc tcaggtggac | 180 |
| ccttcagcgg tgcctactgg acgtggatcc gccaaactcc agggaagggg ctggagtgga | 240 |
| ttggagaggc cggtcggagt ggaaccacca actacaatcc gtccctcaag agtcgagtca | 300 |
| ccatatcact ggacacgtcc aagagccagt tttccctgaa gctgacttcc gtgaccgccg | 360 |
| cggacacggc tgtttacttc tgtgggagac gccaaataat gtctttgagt aatctttata | 420 |
| agagacccgt tgactcttgg ggccggggaa ccccggtcat cgtctcctca gcctccacca | 480 |
| agggcccatc ggtcttcccc ctggcaccct cctccaagag cacctctggg ggcacagcgg | 540 |
| ccctgggctg cctggtcaag gactacttcc ccgaaccggt gacggtgtcg tggaactcag | 600 |
| gcgccctgac cagcggcgtg cacaccttcc cggctgtcct acagtcctca ggactctact | 660 |
| ccctcagcag cgtggtgacc gtgccctcca gcagcttggg cacccagacc tacatctgca | 720 |
| acgtgaatca caagcccagc aacaccaagg tggacaagag agttgagccc aaatcttgtg | 780 |
| acaaaactca cacatgccca ccgtgcccag cacctgaact cctgggggga ccgtcagtct | 840 |
| tcctcttccc cccaa | 855 |
| (36.9F V_{H}) | |
| SEQ ID NO: 6 | |
| gtcactgccc tcggttctat cgattggcta gcaccatgga gacagacaca ctcctgctat | 60 |
| gggtactgct gctctgggtt ccaggttcca ctggtgacga ggtgcagctg gtgcagtctg | 120 |
| gaggaggcct ggtcaaggcg ggggggtccc tgaaactctc ctgtggagcc tctggattca | 180 |
| ccttcagtag ttatagcatg agctgggtcc gccaggctcc agggaagggg ctggagtggg | 240 |
| tctcatacat tagtagtggt gggagttcta tacactacgc agactcagtg aagggccgat | 300 |
| tcaccatctc cagagacaac gccaagaatt cactgtatct gcaaatgaag aacctgaggg | 360 |
| tcgacgacac gggtcggtat tattgtgtga gagatccccg atcggggatc tctggtcggt | 420 |
| acggtatgga cgtctggggt caagggacca cggtcaccgt ctcctcagcc tccaccaagg | 480 |
| gcccatcggt cttccccctg gcaccctcct ccaagagcac ctctgggggc acagcggccc | 540 |
| tgggctgcct ggtcaaggac tacttccccg aaccggtgac ggtgtcgtgg aactcaggcg | 600 |
| ccctgaccag cggcgtgcac accttcccgg ctgtcctaca gtcctcagga ctctactccc | 660 |
| tcagcagcgt ggtgaccgtg ccctccagca gcttgggcac ccagacctac atctgcaacg | 720 |
| tgaatcacaa gcccagcaac accaaggtgg acaagagagt tgagcccaaa tcttgtgaca | 780 |
| aaactcacac atgcccaccg tgcccagcac ctgaactcct ggggggaccg tcagtcttcc | 840 |
| tcttcccccc aaacccaagg acaccctcat gatc | 874 |
| (37.2D V_{H}) | |
| SEQ ID NO: 7 | |
| tcactgccct cggttctatc gattggctag caccatggag acagacacac tcctgctatg | 60 |
| ggtactgctg ctctgggttc caggttccac tggtgacgaa gtgcagctgg tgcagtctgg | 120 |
| agctgaggtg aagaagcctg gggcttcagt gaaggtgtcc tgcaaggcct ctggttacac | 180 |
| ctttacgaaa tacggaatca gctgggtgcg acaggcccct ggacaagggc ttgagtggat | 240 |
| gggatggatc agcgcgttta atggttacac aaggtatggt cagagattcc agggcaaagt | 300 |
| caccatgacc acagacacat ccacgaacac agcctctttg gaggtgagga ccctgacatc | 360 |
| taacgacacg gccgtctatt actgtgcgag acaatatccc gaccaatata gtagcagcgg | 420 |
| ttggccccgc ctcttcgcca tggacgtctg gggccaaggg accacggtca tcgtctcccc | 480 |
| agcctccacc aagggcccat cggtcttccc cctggcaccc tcctccaaga gcacctctgg | 540 |
| gggcacagcg gccctgggct gcctggtcaa ggactacttc cccgaaccgg tgacggtgtc | 600 |
| gtggaactca ggcgccctga ccagcggcgt gcacaccttc ccggctgtcc tacagtcctc | 660 |
| aggactctac tccctcagca gcgtggtgac cgtgccctcc agcagcttgg gcacccagac | 720 |
| ctacatctgc aacgtgaatc acaagcccag caacaccaag gtggacaaga gagttgagcc | 780 |
| caaatcttgt gacaaaactc acacatgccc accgtgccca gcacctgaac tcctgggggg | 840 |
| accgtcagtc ttcctcttc | 859 |
| (37.2G V_{H}) | |
| SEQ ID NO: 8 | |
| tcactgccct cggttctatc gattggctag caccatggag acagacacac tcctgctatg | 60 |
| ggtactgctg ctctgggttc caggttccac tggtgacgag gtgcagctgg tggagtctgg | 120 |
| gggaggcctg gtcaagccgg gggggtcccg gagactctcc tgtgctgcct ctggattcac | 180 |
| cttcagtaga gataccatga cctgggtccg ccaggctcca gggaaggggc tggagtgggt | 240 |
| cgcatccata agtagtggta gcagtgacat aaactacgca gactcagtga agggccgatt | 300 |
| caccatctcc agagacaacg gcaagaactc actgtatctg cacatgaaca gcctgagagc | 360 |
| cgacgacacg gctatatatt actgtgcgag agatccccgg tcgggaatct ctggtcggta | 420 |
| tggtatggac gtctggggcc aagggaccac ggtcaccgtc tcctcagcct ccaccaaggg | 480 |
| cccatcggtc ttccccctgg caccctcctc caagagcacc tctgggggca cagcggccct | 540 |
| gggctgcctg gtcaaggact acttccccga accggtgacg gtgtcgtgga actcaggcgc | 600 |
| cctgaccagc ggcgtgcaca ccttcccggc tgtcctacag tcctcaggac tctactccct | 660 |
| cagcagcgtg gtgaccgtgc cctccagcag cttgggcacc cagacctaca tctgcaacgt | 720 |
| gaatcacaag cccagcaaca ccaaggtgga caagagagtt gagcccaaat cttgtgacaa | 780 |
| aactcacaca tgcccaccgt gcccagcacc tgaactcctg gggggaccgt cagtcttcct | 840 |
| cttcccccca aaacccaagg acaccctcat gatctcccgg acccctgagg tcacatgcgt | 900 |
| ggtggtggac gtgagccacg aagaccctga ggtcaagttc aactggtacg tggacggcgt | 960 |
| (37.7H V_{H}) | |
| SEQ ID NO: 9 | |
| gtcactgcac ctcggttcta tcgattggct agcaccatgg agacagacac actcctgcta | 60 |
| tgggtactgc tgctctgggt tccaggttcc actggtgacg aggtgcagct ggtgcagtct | 120 |
| ggaggaggcc tggtcaaggc gggggggtcc ctgaggctct cctgtgcagc ctccggattc | 180 |
| acattcagca cctacagtat gaactggatc cgccaggctc cagggaaggg gctggagtgg | 240 |
| gtcgcttcca ttagtagtcg aagtggcagt cacataaact acgtagactc agtgaaggga | 300 |
| cgattcacca tctccagaga caacgccagg gacttattgt atctgcaaat gaacagcctg | 360 |
| agagtcgacg actcggctct ctattactgt gcgagagatc gccgttcggg gacttctccc | 420 |
| ctccccttgg acgtctgggg ccaagggacc acggtcaccg tcttctcagc ctccaccaag | 480 |
| ggcccatcgg tcttccccct ggcaccctcc tccaagagca cctctggggg cacagcggcc | 540 |
| ctgggctgcc tggtcaagga ctacttcccc gaaccggtga cggtgtcgtg gaactcaggc | 600 |
| gccctgacca gcggcgtgca caccttcccg gctgtcctac agtcctcagg actctactcc | 660 |
| ctcagcagcg tggtgaccgt gccctccagc agcttgggca cccagaccta catctgcaac | 720 |
| gtgaatcaca agcccagcaa caccaaggtg gacaagagag ttgagcccaa atcttgtgac | 780 |
| aaaactcaca catgcccacc gtgcccagca cctgaactcc tggggggacc gtcagtcttc | 840 |
| ctcttccccc caaaacccaa ggacaccctc atgatctccc ggacccctga ggtcacatgc | 900 |
| gtggtggtgg acgtgagcca cgaa | 924 |
| (8.9F V_{H}) | |
| SEQ ID NO: 10 | |
| cctcggttct atcgattggc tagcaccatg gagacagaca cactcctgct atgggtactg | 60 |
| ctgctctggg ttccaggttc cactggtgac cagggcacct tgagggagtc tggtccagga | 120 |
| ctggtgaggc cttcggagac cctgtccctc acctgcggtg tctctggtta ttccatcagt | 180 |
| agtggttact actggggctg gatccggcag cccccaggga aggggctgga gtggattggg | 240 |
| aatatctatc gtagtgggag cacctactac aacccgtccc tcaagagtcg agtcaccgtc | 300 |
| tcaatagaca cgtccaaaaa ccagttctcc ctgaagttga attctgtgac cgccgcagac | 360 |
| acggccgtgt attactgtgc gagatcgggt ataaaagtgg ctgacgacta ttactacgaa | 420 |
| atggacgtct ggggccaagg gaccgacgac tactcttacg ctatggacgt ctggggccaa | 480 |
| gggaccacgg tcaccgtctc ctcagcctcc accaagggcc catcggtctt ccccctggca | 540 |
| ccctcctcca agagcacctc tgggggcaca gcggccctgg gctgcctggt caaggactac | 600 |
| ttccccgaac cggtgacggt gtcgtggaac tcaggcgccc tgaccagcgg cgtgcacacc | 660 |
| ttcccggctg tcctacagtc ctcaggactc tactccctca gcagcgtggt gaccgtgccc | 720 |
| tccagcagct tgggcaccca gacctacatc tgcaacgtga atcacaagcc cagcaacacc | 780 |
| aaggtggaca agagagttga gcccaaatct tgtgacaaaa ctcacacatg cccaccgtgc | 840 |
| ccagcacctg aactcctggg gggaccgtca gtcttcctct tccccccaaa acccaaggac | 900 |
| accctcatga t | 911 |
| (NE13 V_{H}) | |
| SEQ ID NO: 11 | |
| actgcacctc ggttctatcg attggctagc accatggaga cagacacact cctgctatgg | 60 |
| gtactgctgc tctgggttcc aggttccact ggtgacgagg ttcagctggt ggagtctggg | 120 |
| ggaggcctgg tcaagcctgg ggggtccctg agactctcct gtgtagcctc tggattcacc | 180 |
| ttcagttcct atagcatgaa ctgggtccgc caggctccag ggaaggggct ggagtgggtc | 240 |
| tcatccatta gtagtggtag tagttacata gagtacgcag actcagtgaa gggccgactc | 300 |
| accatctcca gagacaacgc caagaagtca ctgtatctgc aactgaacag cctgagagcc | 360 |
| gaggacacgg ctgtgtatta ctgtgcgaga cacacagctc gaatcgactc ttaccacggt | 420 |
| atggacgtct ggggccaagg gaccacagtc accgtctcct cagcctccac caagggccca | 480 |
| tcggtcttcc ccctggcacc ctcctccaag agcacctctg ggggcacagc ggccctgggc | 540 |
| tgcctggtca aggactactt ccccgaaccg gtgacggtgt cgtggaactc aggcgccctg | 600 |
| accagcggcg tgcacacctt cccggctgtc ctacagtcct caggactcta ctccctcagc | 660 |
| agcgtggtga ccgtgccctc cagcagcttg ggcacccaga cctacatctg caacgtgaat | 720 |
| cacaagccca gcaacaccaa ggtggacaag agagttgagc ccaaatcttg tgacaaaact | 780 |
| cacacatgcc caccgtgccc agcacctgaa ctcctggggg gaccgtcagt cttcctcttc | 840 |
| cccccaaaac ccaaggacac cctcatgatc tcccggaccc c | 881 |
| (12.1F V_{H}) | |
| SEQ ID NO: 12 | |
| gtcactgcac ctcggttcta tcgattggct agcaccatgg agacagacac actcctgcta | 60 |
| tgggtactgc tgctctgggt tccaggttcc actggtgacc aggtgcagct gcaggagtcg | 120 |
| ggcgcaggac tgttgaagcc ttcggagacc ctgtccctca gttgcactgt cgatggtgag | 180 |
| tccttcaatg gtttcttctg gacgtggatc cgccagcccc cagggaaggg tctggagtgg | 240 |
| attggagaaa tcaatcatct tgcaagcacc ggctacaacc cgtccctcaa gagtcgagtc | 300 |
| accatttcag tagacacgtc caagaaccag ttctctttga agttgacctc tgtgaccgcc | 360 |
| gcggacacgg ctgtgtatta ctgtgcgaga ggatacagct atggttttgc atggcccaac | 420 |
| taccactatt tggacgtctg gggcaaaggg accacggtca ccgtctcctc agcctccacc | 480 |
| aagggcccat cggtcttccc cctggcaccc tcctccaaga gcacctctgg gggcacagcg | 540 |
| gccctgggct gcctggtcaa ggactacttc cccgaaccgg tgacggtgtc gtggaactca | 600 |
| ggcgccctga ccagcggcgt gcacaccttc ccggctgtcc tacagtcctc aggactctac | 660 |
| tccctcagca gcgtggtgac cgtgccctcc agcagcttgg gcacccagac ctacatctgc | 720 |
| aacgtgaatc acaagcccag caacaccaag gtggacaaga gagttgagcc caaatcttgt | 780 |
| gacaaaactc acacatgccc accgtgccca gcacctgaac tcctgggggg accgtcagtc | 840 |
| ttcctcttnc ccccaaaacc caaggacacc ctcatgatct cccggacccc tgaggtcaca | 900 |
| tgcgtggtgg tggacgtgag c | 921 |
| (9.8A V_{H}) | |
| SEQ ID NO: 13 | |
| ttctatcgat ttggctagca ccatggagac 9.8A agacacactc ctgctatggg tactgctgct | 60 |
| ctgggttcca ggttccactg gtgacgaggt gcagctggtg cagtctggag gacgcttggt | 120 |
| acagcctggg gggtccctga gactctcctg tgtagcctct ggattcacct ttagcagcca | 180 |
| tgccatgagc tgggtccgcc aggctccagg gaaggggctg gagtgggtct caggttttag | 240 |
| tggtagtagt ggtaccacaa agtacgcaga ctccgtgaag ggccggttca ccatctccag | 300 |
| agacaattcc aagaaaacgc tgtatctgca aatgaacagc ctgagagccg aggacacggc | 360 |
| cgtatattac tgtgcgaaag gcttctcccc atttcgggga gtacaattcc cctactttga | 420 |
| ctactggggc cagggaacgc tggtcaccgt ctcctcagcc tccaccaagg gcccatcggt | 480 |
| cttccccctg gcaccctcct ccaagagcac ctctgggggc acagcggccc tgggctgcct | 540 |
| ggtcaaggac tacttccccg aaccggtgac ggtgtcgtgg aactcaggcg ccctgaccag | 600 |
| cggcgtgcac accttcccgg ctgtcctaca gtcctcagga ctctactccc tcagcagcgt | 660 |
| ggtgaccgtg ccctccagca gcttgggcac ccagacctac atctgcaacg tgaatcacaa | 720 |
| gcccagcaac accaaggtgg acaagagagt tgagcccaaa tcttgtgaca aaactcacac | 780 |
| atgcccaccg tgcccagcac ctgaactcct ggggggaccg tcagtcttcc tcttcccccc | 840 |
| aaaacccagg acaccctcat gatctcccgg accc | 874 |
| (18.5C V_{H}) | |
| SEQ ID NO: 14 | |
| gtccactgca cctcggttct atcgattggc tagcaccatg gagacagaca cactcctgct | 60 |
| atgggtactg ctgctctggg ttccaggttc cactggtgac gaggttcagc tggtggagtc | 120 |
| tgggggaggc ctggtcaggc cgggggggtc ccttagactc tcctgtgcag ccgctggatt | 180 |
| cactttcaag agttatagca tgaattgggt ccgccaggct ccagggaggg gcctggagtg | 240 |
| ggtctcatct atcactagtg gtggtagtaa gacatactat gcagacgtag tgaagggccg | 300 |
| attcaccgtc tccagagaca acgccaagca gtcgctctat ctgcaaatga acagcctgag | 360 |
| agccgaggac acggctatat acttctgtgc gagatcccta catagtacca gccagcctag | 420 |
| ctacatggac gtctggggca gaaagatcac ggtcatcgtc tcctcagcct ccaccaaggg | 480 |
| cccatcggtc ttccccctgg caccctcctc caagagcacc tctgggggca cagcggccct | 540 |
| gggctgcctg gtcaaggact acttccccga accggtgacg gtgtcgtgga actcaggcgc | 600 |
| cctgaccagc ggcgtgcaca ccttcccggc tgtcctacag tcctcaggac tctactccct | 660 |
| cagcagcgtg gtgaccgtgc cctccagcag cttgggcacc cagacctaca tctgcaacgt | 720 |
| gaatcacaag cccagcaaca ccaaggtgga caagagagtt gagcccaaat cttgtgacaa | 780 |
| aactcacaca tgcccaccgt gcccagcacc tgaactcctg gggggaccgt cagtcttcct | 840 |
| cttcccccca aaacccaagg acaccctcat gatctcccgg acccctgagg tcacatgc | 898 |
| (8.11G V_{H}) | |
| SEQ ID NO: 15 | |
| tgcacctcgg ttctatcgat tggctagcac catggagaca gacacactcc tgctatgggt | 60 |
| actgctgctc tgggttccag gttccactgg tgaccaggtg cagctgcagg agtcgggtcc | 120 |
| aggactggtg aagccttcgg agaccctgtc cctcacctgc agtatttctg gtgtgtccac | 180 |
| cagaaattat tattggagct ggatccgcca gtccccaggg aagggactgg agtggattgg | 240 |
| atatatcttt aacattggga ccaccaacta caatccgtcc ctcaagagtc gactcaccat | 300 |
| atctgtagac acgtcgaaga accagttctc cctgaagatc acctctgtga ccgctgcgga | 360 |
| cacggccgtc tattactgtg cgagtggatt tgagtacggt gactatacct tcgactactg | 420 |
| gggccaggga accccggtca ccgtctcctc agcctccacc aagggcccat cggtcttccc | 480 |
| cctggcaccc tcctccaaga gcacctctgg gggcacagcg gccctgggct gcctggtcaa | 540 |
| ggactacttc cccgaaccgg tgacggtgtc gtggaactca ggcgccctga ccagcggcgt | 600 |
| gcacaccttc ccggctgtcc tacagtcctc aggactctac tccctcagca gcgtggtgac | 660 |
| cgtgccctcc agcagcttgg gcacccagac ctacatctgc aacgtgaatc acaagcccag | 720 |
| caacaccaag gtggacaaga gagttgagcc caaatcttgt gacaaaactc acacatgccc | 780 |
| accgtgccca gcacctgaac tcctgggggg accgtcagtc ttcctcttcc ccccaaaacc | 840 |
| caaggacacc ctcatgatct tccggacccc tgaggtcaca tgcgtggtgg tggacgtgag | 900 |
| cca | 903 |
| (25.10C V_{H}) | |
| SEQ ID NO: 16 | |
| ctcggttcta tcgattggct agcaccatgg agacagacac actcctgcta tgggtactgc | 60 |
| tgctctgggt tccaggttcc actggtgacc aggtgcagct gcaggagtct gggggaggcc | 120 |
| tggtcaagcc tggggggtcc ctgagactct cctgtacagc ctctggattc aacttcaata | 180 |
| aatataacat gaactgggtc cgccaggctc cagggaaggg gctggagtgg gtctcatcca | 240 |
| ttagtgctct tagcacttac atctattatg cagactcgct gaagggccga ttcaccgtct | 300 |
| ccagagacaa cgccaagaac tcactgtttc tgcaaatgaa cagcctgaga gacgacgaca | 360 |
| cggctgttta ttactgtgcg agagaaatac gacgtgccag tacctggtcc gccgacctct | 420 |
| ggggccgtgg cactctggtc actgtctcct cagcctccac caagggccca tcggtcttcc | 480 |
| ccctggcacc ctcctccaag agcacctctg ggggcacagc ggccctgggc tgcctggtca | 540 |
| aggactactt ccccgaaccg gtgacggtgt cgtggaactc aggcgccctg accagcggcg | 600 |
| tgcacacctt cccggctgtc ctacagtcct caggactcta ctccctcagc agcgtggtga | 660 |
| ccgtgccctc cagcagcttg ggcacccaga cctacatctg caacgtgaat cacaagccca | 720 |
| gcaacaccaa ggtggacaag agagttgagc ccaaatcttg tgacaaaact cacacatgcc | 780 |
| caccgtgccc agcacctgaa ctcctggggg gaccgtcagt cttcctcttc cctccaaacc | 840 |
| caaggacacc ctcatgatct | 860 |
| (10.4B V_{L}) | |
| SEQ ID NO: 17 | |
| agctgtgacc ggcgcctacc tgagatcacc ggtgctagca ccatggagac agacacactc | 60 |
| ctgctatggg tactgctgct ctgggttcca ggttccactg gtgacgaaat tgtgttgaca | 120 |
| cagtctccat cctcactgtc tgcgtctgta ggagacagag tcaccatcac ttgtcgggcg | 180 |
| agtcgggaca tcaatactta tttaggttgg tttcagcaga gaccagggaa agcccctaag | 240 |
| tccctgatct atggtgcatc taatttgcaa aatggggtcc catcaaggtt cagcggcagt | 300 |
| ggatctggga cgtattttac tctcaccatc aacggcctgc agactgaaga ctttgcgact | 360 |
| tattattgcc aacaatatag catctacccg ctcagtctcg gcggagggac caaggcggac | 420 |
| atgaagcgaa ctgtggctgc accatctgtc ttcatcttcc cgccatctga tgagcagttg | 480 |
| aaatctggaa ctgcctctgt tgtgtgcctg ctgaataact tctatcccag agaggccaaa | 540 |
| gtacagtgga aggtggataa cgccctccaa tcgggtaact cccaggagag tgtcacagag | 600 |
| caggacagca aggacagcac ctacagcctc agcagcaccc tgacgctgag caaagcagac | 660 |
| tacgagaaac acaaagtcta cgcctgcgaa gtcacccatc agggcctgag ctcgcc | 716 |
| (19.7E V_{L}) | |
| SEQ ID NO: 18 | |
| tcagctgtga ccggcgccta cctgagatca ccggtgctag caccatggag acagacacac | 60 |
| tcctgctatg gctcctgctg ctctgggttc caggttccac tggtgacgaa attgtgttga | 120 |
| cacagtctcc ttccaccctg tctgcatctg tgggagacag agtcaccatc acttgccggg | 180 |
| ccagtcagag tattaataat tggttggcct ggtatcagga gaaaccaggg aaagccccta | 240 |
| agctcctgat aaataaggcg tctagtttag aaagtggggt cccatcaagg ttcagcggca | 300 |
| gtggatctgg gacagaattc actctcacca tcaccagcct gcagcctgat gattttgcaa | 360 |
| cttattactg ccaacaatat aatagtaatt cgtggacgtt cggccaaggg accaaggtgg | 420 |
| acatgaaacg aactgtggct gcaccatctg tcttcatctt cccgccatct gatgagcagt | 480 |
| tgaaatctgg aactgcctct gttgtgtgcc tgctgaataa cttctatccc agagaggcca | 540 |
| aagtacagtg gaaggtggat aacgccctcc aatcgggtaa ctcccaggag agtgtcacag | 600 |
| agcaggacag caaggacagc acctacagcc tcagcagcac cctgacgctg agcaaagcag | 660 |
| actacgagaa acacaaagtc tacgcctgcg aagtcaccca tcagggcctg agctcgcccg | 720 |
| tcacaaagag cttcaacagg ggagagtgtt agagggagct agctcgacat gataagatac | 780 |
| attgatgagt ttgggacaac cacaactaga atgcagtgaa aaaaatgctt tatttgtgaa | 840 |
| atttgtgatg ctattgcttt tattgtgaaa tttgtgatgc tattgcttta tttgtaacca | 900 |
| ttataa | 906 |
| (2.9D V_{L}) | |
| SEQ ID NO: 19 | |
| actgcacctc ggttctatcg attggctagc accatgaaga cagacacact cctgctatgg | 60 |
| gtactgctgc tctgggttcc aggttccact ggtgacgaca ttgtgctgac ccagtctcca | 120 |
| gactccctgg ctgtgtctct gggcgagagg gccaccatca actgcaagtc cagccagagt | 180 |
| gttttataca gctccaacaa taagaactac ttagcttggt accagcagaa gccaggacag | 240 |
| cctcctaagc tgctcattta ctgggcatct acccgggaat ccggggtccc tgaccgattc | 300 |
| agtggcagcg ggtctgggac agatttcact ctcaccatca gcagcctgca ggctgaagat | 360 |
| gtggcagttt attactgtca gcaatattat agtactcctc cgacgttcgg ccaagggacc | 420 |
| aaggtggaaa tcaaacgaac tgtggctgca ccatctgtct tcatcttccc gccatctgat | 480 |
| gagcagttga aatctggaac tgcctctgtt gtgtgcctgc tgaataactt ctatcccaga | 540 |
| gaggccaaag tacagtggaa ggtggataac gccctccaat cgggtaactc ccaggagagt | 600 |
| gtcacagagc aggacagcaa ggacagcacc tacagcctca gcagcaccct gacgctgagc | 660 |
| aaagcagact acgagaaaca caaagtctac gcctgcgaag tcacccatca gggcctgagc | 720 |
| tcgcccgtca caaagagctt caacagggga gagtgttagg cggccgcaag cttggccgcc | 780 |
| atggcccaac ttgtttattg cagcttataa tggttacaaa taaagcaata gcatcacaaa | 840 |
| tttcacaaat aaagcatttt tttcactgca ttctagttgt ggtttgtcca actc | 894 |
| (25.6A V_{L}) | |
| SEQ ID NO: 20 | |
| ctcggttcta tcgattggct agcaccatgg agacagacac actcctgcta tgggtactgc | 60 |
| tgctctgggt tccaggttcc actggtgacc tgcctgtgct gactcagcct gcctccgtgt | 120 |
| ctgggtctcc tggacagtcg atcaccatct cctgcactgg aaccagcagt gacgttggtg | 180 |
| cttataacta tgtctcctgg taccaacagc acccaggcaa agcccccaaa ctcataattt | 240 |
| atgaagtcaa gattcggccg tcaggggtgt ctaatcgttt ctctggctcc aagtctggca | 300 |
| acacggcctc cctgaccatc tctgggctcc aggctgagga cgaggctgat tatttttgca | 360 |
| gctcatattc aaccaacagc ccttgggtgt tcggcggagg gacgaaggtg accgtcctac | 420 |
| gtcagcccaa ggctgccccc tcggtcactc tgttcccacc ctcctctgag gagcttcaag | 480 |
| ccaacaaggc cacactggtg tgtctcataa gtgacttcta cccgggagcc gtgacagtgg | 540 |
| cctggaaggc agatagcagc cccgtcaagg cgggagtgga gaccaccaca ccctccaaac | 600 |
| aaagcaacaa caagtacgcg gccagcagct acctgagcct gacgcctgag cagtggaagt | 660 |
| cccacagaag ctacagctgc caggtcacgc atgaagggag caccgtggag aagacagtgg | 720 |
| cccctacaga atgttcatga gcggccgcaa gcttggccgc catggcccaa cttgtttatt | 780 |
| gcagcttata atggttacaa ataaagcaat agcatcacaa atttcacaaa taaagcattt | 840 |
| ttttcactgc attctagttg tggtttgtcc aaactcatca atgtatctta tcatgtctgg | 900 |
| atc | 903 |
| (36.1F V_{L}) | |
| SEQ ID NO: 21 | |
| tccaggtcac tgcacctcgg ttctatcgat tggctagcac catggagaca gacacactcc | 60 |
| tgctatgggt actgctgctc tgggttccag gttccactgg tgacgaaatt gtgctgacac | 120 |
| agtctccagg caccctgtct ttgtctccag gggaaagagc caccctctcc tgcagggcca | 180 |
| gtcagagtgt tactaaaaac tacttagcct ggtaccagca gaaacctggc caggctccca | 240 |
| ccctcgtcat ctatgatgca tccaccaggg ccagtggcat cccagacagg ttcattggca | 300 |
| gtgggtctgg gacagacttc actctcacca tcagcagact ggagcctgaa gattttgcag | 360 |
| tatattactg ccaccagtat ggcagctcac ctccgtacac ttttggccgg gggaccaagc | 420 |
| tggagatcaa acgaactgtg gctgcaccat ctgtcttcat cttcccgcca tctgatgagc | 480 |
| agttgaaatc tggaactgcc tctgttgtgt gcctgctgaa taacttctat cccagagagg | 540 |
| ccaaagtaca gtggaaggtg gataacgccc tccaatcggg taactcccag gagagtgtca | 600 |
| cagagcagga cagcaaggac agcacctaca gcctcagcag caccctgacg ctgagcaaag | 660 |
| cagactacga gaaacacaaa gtctacgcct gcgaagtcac ccatcagggc ctgagctcgc | 720 |
| ccgtcacaaa gagcttcaac aggggagagt gttaggcggc cgcaagcttg gccgccatgg | 780 |
| cccaacttgt ttattgcagc ttataatggt tacaaataaa gcaatagcat cacaaatttc | 840 |
| acaaataaag catttttttc actgcattct agttgtgggt tgtccaaact catcaatgta | 900 |
| (36.9F V_{L}) | |
| SEQ ID NO: 22 | |
| aggtcactgc acctcggttc tatcgattgg ctagcaccat ggagacagac acactcctgc | 60 |
| tatgggtact gctgctctgg gttccaggtt ccactggtga cgacatcgtg atgacccagt | 120 |
| ctccagactc cctggctgtg tctctgggcg agagggccac catcaactgc aagtccagcc | 180 |
| agactgtttt gttcacctcc tattacgtag cttggtatca acaaaagcca gggcagccgc | 240 |
| ctaagttgct cttttccggg gcctcttctc gggaatccgg ggtccctgac cgattcagtg | 300 |
| ccggcgggtc tgggacagat ttctatctca ccatcaacag cctgcaggct gaagatgtgg | 360 |
| cagattacta ttgtcagcaa tatcatactc ctcctttcac tttcggcgga gggaccaagc | 420 |
| tggagatcag acgaactgtg gctgcaccat ctgtcttcat cttcccgcca tctgatgagc | 480 |
| agttgaaatc tggaactgcc tctgttgtgt gcctgctgaa taacttctat cccagagagg | 540 |
| ccaaagtaca gtggaaggtg gataacgccc tccaatcggg taactcccag gagagtgtca | 600 |
| cagagcagga cagcaaggac agcacctaca gcctcagcag caccctgacg ctgagcaaag | 660 |
| cagactacga gaaacacaaa gtctacgcct gcgaagtcac ccatcagggc ctgagctcgc | 720 |
| ccgtcacaaa gagcttcaac aggggagagt gttaggcggc cgcaagcttg gccgccatgg | 780 |
| cccaacttgt ttattgcagc ttataatggt tacaaataaa gcaatagcat cacaaatttc | 840 |
| acaaataaag catttttttc actgcattct agttgtggtt tgtccaaact catcaatgta | 900 |
| tcttatcatg tctggatcgg ga | 922 |
| (37.2D V_{L}) | |
| SEQ ID NO: 23 | |
| tcactgcacc tcggttctat cgattggcta gcaccatgga gacagacaca ctcctgctat | 60 |
| gggtactgct gctctgggtt ccaggttcca ctggtgacga aacgacactc acgcagtctc | 120 |
| cagccaccct gtctgtgtct ccaggggaaa cagccaccct ctcctgcagg gccagtcaaa | 180 |
| atgttatcaa caacttagcc tggtaccagc agaaacctgg ccaggctccc aggctcctca | 240 |
| tttatggtgc atccaccagg gccactggta tcccagccag gttcagtggc agtgggtctg | 300 |
| ggacagagtt cactctcacc atcagcagca tgcagtctga agattttgca gtttattact | 360 |
| gtcagcaata taatgactgg cctcgaagtt ttggccaggg gaccaggctg gacatcagac | 420 |
| gaactgtggc tgcaccatct gtcttcatct tcccgccatc tgatgagcag ttgaaatctg | 480 |
| gaactgcctc tgttgtgtgc ctgctgaata acttctatcc cagagaggcc aaagtacagt | 540 |
| ggaaggtgga taacgccctc caatcgggta actcccagga gagtgtcaca gagcaggaca | 600 |
| gcaaggacag cacctacagc ctcagcagca ccctgacgct gagcaaagca gactacgaga | 660 |
| aacacaaagt ctacgcctgc gaagtcaccc atcagggcct gagctcgccc gtcacaaaga | 720 |
| gcttcaacag gggagagtgt taggcggccg caagcttggc cgccatggcc caacttgttt | 780 |
| attgcagctt ataatggtta caaataaagc aatagcatca caaatttcac aaataaagca | 840 |
| tttttttcac tgcattct | 858 |
| (37.2G V_{L}) | |
| SEQ ID NO: 24 | |
| tccaggtcac tgccctcggt tctatcgatt ggctagcacc atggagacag acacactcct | 60 |
| gctatgggta ctgctgctct gggttccagg ttccactggt gacgacattg tgctgaccca | 120 |
| gtctccaggc accctgtctt tgtctccagg ggaaagagcc accctctcct gcagggccag | 180 |
| tcagagtgtg aacagcatct tcttagcctg gtaccagcag aaacctggcc aggctcccag | 240 |
| gctcctcatc tatggtgcat ccagcagggc cactggcatc ccagacaggt tcagtggcag | 300 |
| tgggtctggg acagacttca ctctcaccat cagcagactg gagcctgagg attttgcagt | 360 |
| gtattactgt cagcagtatc atagctcacc taagctcact ttcggcggag ggaccaaggt | 420 |
| ggagatcaaa cgaactgtgg ctgcaccatc tgtcttcatc ttcccgccat ctggtgagca | 480 |
| gttgaaatct ggaactgcct ctgttgtgtg cctgctgaat aacttctatc ccagagaggc | 540 |
| caaagtacag tggaaggtgg ataacgccct ccaatcgggt aactcccagg agagtgtcac | 600 |
| agagcaggac agcaaggaca gcacctacag cctcagcagc accctgacgc tgagcaaagc | 660 |
| agactacgag aaacacaaag tctacgcctg cgaagtcacc catcagggcc tgagctcgcc | 720 |
| cgtcacaaag agcttcaaca ggggagagtg ttaggcggcc gcaagcttgg ccgccatggc | 780 |
| ccaacttgtt tattgcagct tataatggtt acaaataaag caatagcatc acaaatttca | 840 |
| caaataaagc atttttttca ctgcattcta gttgtggttt gtccaaactc atcaatgtat | 900 |
| cttatcatgt ctggatcggg aattaattcg gcgcagcacc atggcctgaa ataacctc | 958 |
| (37.7H V_{L}) | |
| SEQ ID NO: 25 | |
| tcactgcacc tcggttctat cgattggcta gcaccatgga gacagacaca ctcctgctat | 60 |
| gggtactgct gctctgggtt ccaggttcca ctggtgacca gtctgccctg actcagcctg | 120 |
| cctccgtgtc tgggtctcct ggacagtcga tcaccatctc ctgcactgga accggcagtg | 180 |
| acattggtgg ttataacttt gtctcctggt accaacagta tcccggcaaa gcccccaaac | 240 |
| tcattattta tgaggtccgt attcgggcct caggggtttc caatcgcttc tctggctcca | 300 |
| agtctggcaa cacggcctcc ctgaccatct ctggactcca ggctgaggac gaggctgatt | 360 |
| attactgcaa ctcatattca atccacagcc cttgggtgtt cggcggaggg accaagttga | 420 |
| ccgtcctgcg tcagcccaag gctgccccct cggtcactct gttcccaccc tcctctgagg | 480 |
| agcttcaagc caacaaggcc acactggtgt gtctcataag tgacttctac ccgggagccg | 540 |
| tgacagtggc ctggaaggca gatagcagcc ccgtcaaggc gggagtggag accaccacac | 600 |
| cctccaaaca aagcaacaac aagtacgcgg ccagcagcta cctgagcctg acgcctgagc | 660 |
| agtgggagtc ccacagaagc tacagctgcc aggtcacgca tgaagggagc accgtggaga | 720 |
| agacagtggc ccctacagaa tgttcatgag cggccgcaag cttggccgcc atggcccaac | 780 |
| ttgtttattg cagcttataa tggttacaaa taaagcaata gcatcacaaa tttcacaaat | 840 |
| aaagcatttt tttcactgca ttctagttgt ggtttgtcca aactcatcaa tgtatcttat | 900 |
| catgtctgga tcgggaatta attcggcgca gcaccatggc ctgaaatacc ctctgaaaga | 960 |
| ggaacttggt taggtacctt ctgaggcgga aagaaccatc tgtggaatgt gtgtc | 1015 |
| (8.9F V_{L}) | |
| SEQ ID NO: 26 | |
| cactgccctc ggttctatcg attggctagc accatggaga cagacacact cctgctatgg | 60 |
| gtactgctgc tctgggttcc aggttccact ggtgaccagg cagggctgac tcagcctgcc | 120 |
| tccgtgtctg ggtctcctgg acagtcgatc accatctcct gcactgcagc caacagtgac | 180 |
| attggtgatt ttaactttgt ctcctggtac caacagcgcc cagacaaagc ccccaaactc | 240 |
| atggtttatg aggtcagcag tcggccctca ggggtttcta atcgcttctc tggctccaag | 300 |
| tctggcaaca cggcctccct gaccatctct gggctccagg ctgaggacga ggctgattat | 360 |
| tactgcacct catatacaag cagcagcact tttgtcttcg gaactgggac caaggtcacc | 420 |
| gtcctaggtc agcccaaggc caaccccact gtcactctgt tcccgccctc ctctgaggag | 480 |
| cttcaagcca acaaggccac actggtgtgt ctcataagtg acttctaccc gggagccgtg | 540 |
| acagtggcct ggaaggcaga tagcagcccc gtcaaggcgg gagtggagac caccacaccc | 600 |
| tccaaacaaa gcaacaacaa gtacgcggcc agcagctacc tgagcctgac gcctgagcag | 660 |
| tggaagtccc acagaagcta cagctgccag gtcacgcatg aagggagcac cgtggagaag | 720 |
| acagtggccc ctacagaatg ttcatgagcg gccgcaagct tggccgccat ggcccaactt | 780 |
| gtttattgca gcttataatg gttacaaata aagcaatagc atcacaaatt tcacaaataa | 840 |
| agcatttttt tcactgcatt ctagttgtgg tttgtccaaa ctcatcaatg tatcttatca | 900 |
| tgtctggatc | 910 |
| (NE13 V_{L}) | |
| SEQ ID NO: 27 | |
| ctcccaggtc actgcacctc ggttctatcg attggctagc accatggaga cagacacact | 60 |
| cctgctatgg gtactgctgc tctgggttcc aggttccact ggtgacgaaa cgacactcac | 120 |
| gcagtctcca ggcaccctgt ctttgtctcc aggggaaaga gccaccctct cctgcagggc | 180 |
| cagtcagagt gttagcagca cctacttagc ctggtaccag cagaaacctg gccagtctcc | 240 |
| caggctcctc atttatggtg catccagtag ggccactggc atcccagaca ggttcagtgg | 300 |
| cagtgggtct gggacacagt tcactctcac catcaacaga ctggagcctg aagattttgc | 360 |
| agtgtattac tgtcagcagt ttggtagccc gtggacattc ggccaaggga ccaaggtgga | 420 |
| aatcaaacga actgtggctg caccatctgt cttcatcttc ccgccatctg atgagcagtt | 480 |
| gaaatctgga actgcctctg ttgtgtgcct gctgaataac ttctatccca gagaggccaa | 540 |
| agtacagtgg aaggtggata acgccctcca atcgggtaac tcccaggaga gtgtcacaga | 600 |
| gcaggacagc aaggacagca cctacagcct cagcagcacc ctgacgctga gcaaagcaga | 660 |
| ctacgagaaa cacaaagtct acgcctgcga agtcacccat cagggcctga gctcgcccgt | 720 |
| cacaaagagc ttcaacaggg gagagtgtta ggcggccgca agcttggccg ccatggccca | 780 |
| acttgtttat tgcagcttat aatggttaca aataaagcaa tagcatcaca aatttcacaa | 840 |
| ataaagcatt tttttcactg cattctagtt gtggtttgtc caaactcatc aatgtatctt | 900 |
| atcatgtc | 908 |
| (12.1F V_{L}) | |
| SEQ ID NO: 28 | |
| gtcactgcac ctcggttcta tcgattggct agcaccatgg agacagacac actcctgcta | 60 |
| tgggtactgc tgctctgggt tccaggttcc actggtgacg aaacgacact cacgcagtct | 120 |
| ccagccaccc tgtctttgtc tccaggggag agagccaccc tctcctgtag ggccagtcag | 180 |
| agtgttagca gctacttagc ctggtaccaa cacaaacctg gccaggctcc caggctcctc | 240 |
| atctatggtg catcaaagag ggccactggc atcccgtcca ggttcagtgg cagtgggtct | 300 |
| gggacagact tcagtctcac catcagcagc ctagagcctg aagattttgc agtttactac | 360 |
| tgtcagcacc gaagcgactg gcggactacc ttcggccaag ggacacgact ggagattaaa | 420 |
| cgaactgtgg ctgcaccatc tgtcttcatc ttcccgccat ctgatgagca gttgaaatct | 480 |
| ggaactgcct ctgttgtgtg cctgctgaat aacttctatc ccagagaggc caaagtacag | 540 |
| tggaaggtgg ataacgccct ccaatcgggt aactcccagg agagtgtcac agagcaggac | 600 |
| agcaaggaca gcacctacag cctcagcagc accctgacgc tgagcaaagc agactacgag | 660 |
| aaacacaaag tctacgcctg cgaagtcacc catcagggcc tgagctcgcc cgtcacaaag | 720 |
| agcttcaaca ggggagagtg ttaggcggcc gcaagcttgg ccgccatggc ccaacttgtt | 780 |
| tattgcagct tataatggtt acaaataaag caatagcatc acaaatttca caaataaagc | 840 |
| atttttttca ctgcattcta gttgtggttt gtccaaactc atcaatgtat cttatcatgt | 900 |
| ctggatcggg aaattaatcg gcgcagcacc at | 932 |
| (9.8A V_{L}) | |
| SEQ ID NO: 29 | |
| ggttctatcg attggctagc accatggaga cagacacact cctgctatgg gtactgctgc | 60 |
| tctgggttcc aggttccact ggtgacgaca tcgtgatgac ccagtctcct tccaccctgt | 120 |
| ctgcatctgt aggagacaga gtcaccatca cttgccgggc cagtcagagt attgataggt | 180 |
| ggttggcctg gtatcagcag aaaccaggga aagcccctaa gctcctgatc tatcaggcat | 240 |
| ctagtttaga aagaggggtc ccatcaaggt tcagcggcag tggatctggg acagaattca | 300 |
| ctctcaccat cagcagcctg cagcccgatg attttgcaac ttattactgc caacagtata | 360 |
| atggttaccc tctcactttc ggcggaggga ccaaggtgga gatcaaacga actgtggctg | 420 |
| caccatctgt cttcatcttc ccgccatctg atgagcagtt gaaatctgga actgcctctg | 480 |
| ttgtgtgcct gctgaataac ttctatccca gagaggccaa agtacagtgg aaggtggata | 540 |
| acgccctcca atcgggtaac tcccaggaga gtgtcacaga gcaggacagc aaggacagca | 600 |
| cctacagcct cagcagcacc ctgacgctga gcaaagcaga ctacgagaaa cacaaagtct | 660 |
| acgcctgcga agtcacccat cagggcctga gctcgcccgt cacaaagagc ttcaacaggg | 720 |
| gagagtgtta ggcggccgca agcttggccg ccatggccca acttgtttat tgcagcttat | 780 |
| aatggttaca aataaagcaa tagcatcaca aatttcacaa ataaagcatt tttttcactg | 840 |
| cattctagtt gtggtttgtc caaactcatc aatgtatctt atcatgtctg gatcg | 895 |
| (18.5C V_{L}) | |
| SEQ ID NO: 30 | |
| tccaggtcca ctgcacctcg gttctatcga ttggctagca ccatggagac agacacactc | 60 |
| ctgctatggg tactgctgct ctgggttcca ggttccactg gtgacgacat ccagatgacc | 120 |
| cagtctccag gcaccctgtc tttgtctcca ggggaaagag ccaccctctc ctgcagggcc | 180 |
| agtcagagtg ttatcagtta ctacgtagcc tggtaccagc acaaaggtgg ccaggctccc | 240 |
| aggctcctca tttatggtgc atccagcagg gccactggcg tcccagacag gttcagtggc | 300 |
| agtgggtctg ggacagactt cactctcacc atcagcagcc tggagcctga agattttgca | 360 |
| ctgtattact gtcagtacta tgggagctca cctctgtggg cgttcggcca agggaccaag | 420 |
| gtggaaatca aacgaactgt ggctgcacca tctgtcttca tcttcccgcc atctgatgag | 480 |
| cagttgaaat ctggaactgc ctctgttgtg tgcctgctga ataacttcta tcccagagag | 540 |
| gccaaagtac agtggaaggt ggataacgcc ctccaatcgg gtaactccca ggagagtgtc | 600 |
| acagagcagg acagcaagga cagcacctac agcctcagca gcaccctgac gctgagcaaa | 660 |
| gcagactacg agaaacacaa agtctacgcc tgcgaagtca cccatcaggg cctgagctcg | 720 |
| cccgtcacaa agagcttcaa caggggagag tgttaggcgg ccgcaagctt ggccgccatg | 780 |
| gccc | 784 |
| (8.11G V_{L}) | |
| SEQ ID NO: 31 | |
| cggttctatc gattggctag caccatggag acagacacac tcctgctatg ggtactgctg | 60 |
| ctctgggttc caggttccac tggtgacgaa attgtgctga ctcagtctcc agccaccctg | 120 |
| tctgtgtctc cagggggtag ggcctccctc tcctgccggg ccagtcagag tattggcgac | 180 |
| aagttatcct ggtatcagca gaaacctggg caggctccca ggctcgtcat ctatggtgca | 240 |
| tataccaggg ccactgatat ctcacccagg ttcagtggca gtaggtctgg gacagacttc | 300 |
| aatctcacca tcagcagaat gcagtctgga gactttgcag tttatttctg tcagcagtat | 360 |
| gaaaactggc ctcggacttt tggccagggg accaagctgg agatcaaacg aactgtggct | 420 |
| gcaccatctg tcttcatctt cccgccatct gatgagcagt tgaaatctgg aactgcctct | 480 |
| gttgtgtgcc tgctgaataa cttctatccc agagaggcca aagtacagtg gaaggtggat | 540 |
| aacgccctcc aatcgggtaa ctcccaggag agtgtcacag agcaggacag caaggacagc | 600 |
| acctacagcc tcagcagcac cctgacgctg agcaaagcag actacgagaa acacaaagtc | 660 |
| tacgcctgcg aagtcaccca tcagggcctg agctcgcccg tcacaaagag cttcaacagg | 720 |
| ggagagtgtt aggcggccgc aagcttggcc gccatggccc aacttgttta ttgcagctta | 780 |
| taatggttac aaataaagca atagcatcac aaatttcaca aataaagcat ttttttcact | 840 |
| gcatt | 845 |
| (25.10C V_{L}) | |
| SEQ ID NO: 32 | |
| cctcggttct atcgattggc tagcaccatg gagacagaca cactcctgct atgggtactg | 60 |
| ctgctctggg ttccaggttc cactggtgac gacatccaga tgacccagtc tccatcctcc | 120 |
| ctgtctgcat ctgttggaga cagagtcatc atcacttgcc gggcaagtca gagcatcagc | 180 |
| agctctttaa attggtatca gcagaaacca gggaaagccc ctaagctcct gatctatgct | 240 |
| gcagtcaatt tggagactgg ggtcccgtca aggttcagtg gcagtggatt tgggacagat | 300 |
| ttcactctcg ccatcagcaa tgtgcaacct gaagattttg caacttacta ctgtcaacag | 360 |
| agcgatactc ggacttttgg ccgggggacc aagctggacg tcaaacgaac tgtggctgca | 420 |
| ccatctgtct tcatcttccc gccatctgat gagcagttga aatctggaac tgcctctgtt | 480 |
| gtgtgcctgc tgaataactt ctatcccaga gaggccaaag tacagtggaa ggtggataac | 540 |
| gccctccaat cgggtaactc ccaggagagt gtcacagagc aggacagcaa ggacagcacc | 600 |
| tacagcctca gcagcaccct gacgctgagc aaagcagact acgagaaaca caaagtctac | 660 |
| gcctgcgaag tcacccatca gggcctgagc tcgcccgtca caaagagctt caacagggga | 720 |
| gaagtgttag gcggccgcaa gcttggccgc catggcccaa cttgtttatt gcagcttata | 780 |
| atggttacaa ataaagcaat agcatcacaa atttcacaaa taaagcattt ttttcactgc | 840 |
| attctagttg tggtttgtcc aaactcatca atgtatctta tcatgtctgg atcgggaatt | 900 |

### Antibody Amino Acid Sequences

The V_{H} and V_{L} amino acid sequences of the antibodies and complementarity determining regions (CDR) of the V_{H} and V_{L} sequences are shown and discussed below.

(10.4B V_{H})
SEQ ID NO: 33
METDTLLLWVLLLWVPGSTGDQVQLVQSGGGVVQPGRSLRVSCVISGFNF
RAYGMHWVRQIPGKGLEWVADIWSAETNRHYADSVKGRFTISRDNSKSTL
YLQMNSIRAEDIGVYFCAKARPGYDYVVDLWGQGTLVIVSSASTKGPSVF
PLAPCSRSTSGGTAPLLGCLVKDYFPEPVTVSWNSGALTSGVHTFPAVLQ
SSGL
(19.7E V_{H})
SEQ ID NO: 34
METDTLLLWVLLLWVPGSTGDEVQLVESGGGIVRPGGSLRLSCAASGYSF
ESYSMHWVREVPGKGINWVSYINSDGSTKIYADSVKGRFSISRDNAKNKL
YLQMDSLRVEDTAVYSCVRLVHYDWSPFVWGQGTLVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKKVEPQSCDKTHTCP
PCPAPELL
(2.9D V_{H})
SEQ ID NO: 35
METDTLLLWVLLLWVPGSTGDEVQLVESGGGLVKPGGSLRLSCAASGFTF
TRFTLTWVRQAPGKGLEWVSSISSGSSDINYADSVKGRFTISRDNARNSL
FLQMSSLRVDDTAVYYCAKDPRSGISGRYGMDVWGQGTTVIVSSASTKGP
SVFPLAPCSRSTSGGIAALGCLVKDYFPEPVTVEWNSGALTSGVHTYPAV
LQSSGLYSLSSVVTVPSSSLGTQTYICNVNHKPSNTKVDKRVEPKSCDKT
HTCPPCPAPELLGGPSVFLFPPKPKDTLMISRIPEVTCVVVDVS
(25.6A V_{H})
SEQ ID NO: 36
METDTLLLWVLLLWVPGSTGDQVQLQESGGGLVKAGGSLRLSCAASGFMF
ERYSLHWVRQTPGKGLEWVSSISSLSGSHINYADSVKGRFTISRDNAKNS
LSLQMNSLBVEDTAIYYCARDRRSGSSPVPLDVWGQGTTVTVSSASTKGP
SVFPLAPSSKSTSGGTAALGC
(36.1F V_{H})
SEQ ID NO: 37
METDTLLLWVLLLWVPGSTGDQVQLQESGAGIVKPSETLSLTCAVSGGPF
SGAYWTWIRQTPGKGLEWIGEAGRSGTTNYNPSLKSRVTISLDISKSQFS
LKLTSVTAADTAVYFCGRRQIMSLSNLYKRPVDSWGRGTPVIVSSASTKG
PSVFPLAPSSKSTSGGTAALGCLVKDYFPEPVTVSWNSGALTSGVHTYPA
VLQSSGLYSLSSVVTVPSSSLGTQTYICNVNHKPSNTKVDKRVEPKSCDK
THTCPPCPAPELLGGPSVFLFPPX
(36.9F V_{H})
SEQ ID NO: 38
METDTLLLTWVLLLWVPGSTGDEVQLVQSGGGLVKAGGSLKLSCGASGFT
FSSYSMSWVRQAPGKGLEWVSYISSGGSSIHYADSVKGRFTISRDNAKNS
LYLQMKNLRVDDTGRYYCVRDPRSGISGRYGMDVWGQGTTVTVSSASTKG
PSVFPLAPSSKSTSGGTAALGCLVKDYFPEPVTVSWNSGALTSGVETFPA
VLQSSGLYSLSSVVTVPSSSLGTQTYICNVNHKPSNTKVDKRVEPKSCDK
THTCPPCDAPELLGGPSVFLPPNPRTPS*S
(37.2D V_{H})
SEQ ID NO: 39
METDTLLLWVLLLWVPGSTGDEVQLVQSGAEVKKPGASVKVSCKASGYTF
TKYGISWVRQAPGQGLEWMGWISAFNGYTRYGQRFQGKVTMTTDTSTNTA
SLEVRTLTSNDTAVYYCARQYPDQYSSSGWPRIFAMDVWGQGTTVIVSPA
STKGPSVFPLAPSSKSTSGGTAALGCLVKDYFPEPVTVEWNSGALTSGVH
TFPAVLQSSGLYSLSSVVTVPSSSLGTQTYICNVNHKPSNTKVDKRVEPK
SCDKTHTCPPCPAPELLGGPSVFLF
(37.2G V_{H})
SEQ ID NO: 40
METDTLLLWVLLLWVPGSTGDEVQLVESGGGLVKPGGSRRLSCAASGFTF
SRDTMTWVRQAPGKGLEWVASISSGSSDINYADSVKGRFTISRDNGKNSL
YLHMNSLRADDTAIYYCARDPRSGISGRYGMDVWGQGTTVTVSSASTKGP
SVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKT
HTCPPCPAPELLGGPSVFLFPPKPKDTLMISRTPEVTCVVVDVSHEDPEV
KFNWYVDGV
(37.7H V_{H})
SEQ ID NO: 41
METDTLLLWVLLLWVPGSTGDEVQLVQSGGGLVKAGGSLRLSCAASGFTF
STYSMNWIRQAPGKGLEWVASISSRSGSHINYVDSVKGRFTISRDNARDL
LYLQMNSLRVDDSALYYCARDRRSGTSPLPLDVWGQGTTVTVFSASTKGP
SVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKT
HTCPPCPAPELLGGPSVFLFPPKPKDTLMISRTPEVTCVVVDVSHE
(8.9F V_{H})
SEQ ID NO: 42
METDTLLLWVLLLWVPGSTGDQGTLRESGPGLVRPSETLSLTCGVSGYSI
SSGYYNGWIRQPPGKGLEWIGNIYRSGSTYYNPSLKERVTVSIDTSKNQF
SLKLNSVTAADTAVYYCARSGIKVADDYYYEMDVWGQGTDDYSYAMDVWG
QGTTVTVSSASTKGPSVFPLAPSSKSTSGGTAALGCLVKDYETEPVTVSW
NSGALTSGVHTFPAVLQSSGLYSLSSVVTVPSSSLGTQTYICNVNHKPSN
TKVDKRVEPKSCDKTHTCPPCPAPELLGGPSVFLFPPKPKDTLMX
(NE13 V_{H})
SEQ ID NO: 43
METDTLLLWVLLLWVPGSTGDEVQLVESGGGLVKPGGSLRLSCVASGFTF
SSYSMNWVRQAPGKGLEWVSSISSGSSYIEYADSVKGRLTISRDNAKKSL
YLQLNSLRAEDTAVYYCARHTARIDSYHGMDVWGQGTTVTVSSASTKGPS
VFPLAPSSKSTSGGTAALGCLVKDYFPEPVTVSWNSGALTSGVHTFPAVL
QSSGLYSLSSVVTVPSSSLGTQTYICNVNHKPSNIKVDKRVEPKSCDKTH
TCPPCPAPELLGGPSVFLFPPKPKDTLMISRTP
(12.1F V_{H})
SEQ ID NO: 44
METDTLLLWVLLLWVPGSTGDQVQLQESGAGLLKPSETLSLSCTVDGESF
NGFFWTWIRQPPGKGLEWIGEINHLASTGYNPSLKSRVTISVDTSKNQFS
LKLTSVTAADTAVYYCARGYSYCFAWPNYHYLDVWGKGTTVTVSSASTKG
PSVFPLAPSSKSTSGGTAALGCLVKDYYPEPVTVWNSGALTSGVHTFPAV
ILQSSGLYSLSSVVTVPSSSLGTQTYICNVNHKPSNTKVDKRVEPKSCDK
THTCPPCPAPELLGGPSVFLXDPKPKDTLMISRTPEVTCVVVDVS
(9.8A V_{H})
SEQ ID NO: 45
METDTDLLLWVLLLWVPGSTGDEVQLVQSGGRLVQPGGSLRLSCVASGFT
FSSHAMSWVRQAPGKGLEWVSGFSGSSGTIKYADSVKGRFTISRDNSKKT
LYLQMNSLRAEDTAVYYCAKGFSPFRGVQFPYFDYWGQGTLVTVSSASTK
GPSVFPLAPSSKSTSGGTAALGCLVKDYTPEPVTVSWNSGALTSGVHTFP
AVLQSSGLYSLSSVVTVPSSSLGTQTYICNVNHKPSNTKVDKRVEPKSCD
KTHTCPPCPAPELLGGPSVFLFDPKPRIPS*SPGP
(18.5C V_{H})
SEQ ID NO: 46
METDTMLLWVLLLWVPGSTGDRVQLVESGGCLVRPGGSLRLSCAAAGFTF
KSYSMNWVRQAPGRGLEWVSSITSGGSKTYYADVVKGRFTVSRDNAKQSL
YLQMNSLRAEDTAIYFCARSLHSTSQPSYMDVWGRKITVIVSSASTKGPS
VFPLAPSSKSTSGGTAALGCLVKDYFPEPVTVSWNSGALTSGVHTFPAVL
QSSGLYSLSSVVTVPSSSLGTQTYICNVNHKPSNTKVDKRVEPKSCDKTH
TCPPCPAPELLGGPSVFLFPPKPKDTLMISRTPRVTC
(8.11G V_{H})
SEQ ID NO: 47
METDTLLLWVLLLWVPGSTGDQVQLQESGPGLVKPSETLSLTCSISGVST
RNYYWSWIRQSPGKGLEWIGYIFNIGTTNYNPSLKSRLTISVDTSKNQFS
LKITSVTAADTAVYYCASGFEYGDYTFDYWGQGTPVTVSSASTKGPSVET
LAPSSKSTSGGTAALGCLVKDYFPEPVTVEWNSGALTSGVHTYPAVLQSS
GLYSLSSVVTVPSSSLGTQTYICNVNHKPSNTKVDKRVEPKSCDKTHTCP
PCPAPELLGGPSVFLFPPKPKDTLMIFRTPEVTCVVVDVS
(25.10C V_{H})
SEQ ID NO: 48
METDTLLLWVLLLWVPGSTGDTQLQESGGGLVKPGGSLRLSCTASGFNFN
KYNKNWVRQAPGKGLEWVSSISALSTYIYYADSLKGRFTVSRDNAKNSLF
LQMNSLRDDDTAVYYCAREIRRASTWSADLWGRGTLVTVSSASTKGPSVF
PLAPSSKSTSGGTAALGCLVKDYFPEPVTVSWNSGALTSGVHTFPAVLQS
SGLYSLSSVVTVPSSSLGTQTYICNVNHKPSNTKVDKRVEPKSCDKTHTC
PPCPAPELLGGPSVFLFPPNPRTPS
(10.4B V_{L})
SEQ ID NO: 49
METDTLLLWVLLLWVPGSTGDEIVLTQSPSSLSASVGDRVTITCRASRDI
NTYLGWFQQRPGKAPKSLIYGASNLQNGVPSRFSGSGSGTYFTLTINGLQ
TEDFATYYCQQYSIYPLSLGGGTKADMKRTVAAPSVFIFPPSDEQLKEGT
ASVVCLLNNFYPREAKVQWKVDNALQSGNSQESVTEQDSKDSTYSLSSTL
TLSKADYFKHKVYACEVTHQGLSSP
(19.7E V_{L})
SEQ ID NO: 50
METDTLLLWLLLLWVPGSTGDEIVLTQPSTLSASVGDRVTITCRASQSIN
NWLAWYQEKPGKAPKLLINKASSLESGVPSRFSGSGSGTEFTLTITSLQP
DDFATYYCQQYNSNSWTFGQGTKVDMKRTVAAPSVFIFPPSDEQLKSGTA
SVVCLLNNFYPREAKVQWKVDNALQSGNESQESVTEQSKDSTYSLSSTLT
LSKADYEKHKVYACEVTHQGLSSPVTKSFNRGEC
(2.9D V_{L})
SEQ ID NO: 51
MKTDTLLLWVLLLWVPGSTGDDIVLTQSPDSLAVSLGERATINCKSSQSV
LYSSNNKNYLAWYQQKPGQPPKLLIYWASTRESGVPDRFSGSGSGTDFTL
TISSLQAEDVAVYYCQQYYSTPPTFGQGTKVEIKRTVAAPSVFIFPPSDE
QLKSGTASVVCLLNNTYPREAKVQWKVDNALQSGNSQESVTFQDSKDSTY
SLSSTLTLSKADYEKHKVYACEVTHQGLSSPVTKSFNRGEC
(25.6A V_{L})
SEQ ID NO: 52
METDTLLLWVLLLWVPGSTGDLPVLTQPASVSGSPGQSITISCTGTSSDV
GAYNYVSWYQQHPGKAPKLIIYEVKIRPSGVSNRFSGSKSGNTASLTISG
LQAEDEADYFCSSYSTNSPWVFCGGTKVTVLRQPKAAPSVTLFPPSSEEL
QANKATLVCLISDFYPCAVTVAWKADSSPVKAGVETTTPSKQSNNKYAAS
SYLSLTPEQWKSHRSYSCQVTHEGSTVEKTVAPTECS*
(36.1F V_{L})
SEQ ID NO: 53
METDTLLLWVLLLWVPGSTGDEIVLTQSPGTLSLSPGERATLSCRASQSV
TKNYLAWYQQKPGQAPTLVIYDASTRASGIPDRFIGSGSGTDFTLTISRL
EPEDFAVYYCHQYGSSPPYTFGRGTKLEIKRTVAAPSVFIFPPSDEQLKS
GTASVVCLLNNEYPREAKVQWKVDNALQSGNSQESVTEQDSKDSTYSLSS
TLTLSKADYEKHKVYACEVTHQGLSSPVTKSFNRGEC*
(36.9F V_{L})
SEQ ID NO: 54
METDTLLLWVLLLWVPGSTGDDIVMTQSPDSLAVSLGERATINCKSSQTV
LFTSYYVAWYQQKPGQPPKLLFSGASSRESGVPDRESAGGSGTDFYLTIN
SLQAEDVADYYCQQYHTPPFTFGGGTKLEIRRTVAAPSVFIFPPSDEQLK
SGTASVVCLLNNFYPREAKVQWKVDNALQSGNSQESVTEQDSKDSTYSLS
SILTLSKADYEKHKVYACEVTHQGLSSPVTKEFNRGEC*
(37.2D V_{L})
SEQ ID NO: 55
METDTLLLWVLLLWVPGSTGDETTLTQSPATLSVSPGETATISCRASQNV
INNLAWYQQKPGQAPRLLIYGASTRATGIPARFSGSGSGTEFTLTISSMQ
SEDFAVYYCQQYNDWPRSFGQGTRLDIRRTVAAPSVFIFPPSDEQLKSGT
ASVVCLLNNEYPREAKVQWKVDNALQSGNSQESVTEQDSKDSTYSLSSTL
TLSKADYEKHKVYACEVTHQGLSSPVTKSFNRGEC*
(37.2G V_{L})
SEQ ID NO: 56
METDTLLLWLLLWVPGSTGDDIVLTQSPGTLSLSPGERATLSCRASQSVN
SIFLAWYQQKPGQAPRLLIYGASSRATGIPDRFSGSGSGTDFTLTISRLE
PEDFAVYYCQQYHSSPKLTFGGGTKVEIKRTVAAPSVFIFPPSGEQLKSG
TASVVCLLNNFYPREAKVQWKVDNALQSGNSQESVTEQDSKDSTYSLSST
LTLSKADYEKHKVYACEVTHQGLSSPVTKSFNRGEC*
(37.7H V_{L})
SEQ ID NO: 57
METDTLLLWVLLLWVPGSTGDQSALTQPASVSGSPGQSITISCTGTGSDI
GGYNFVSWYQQYPGKAPKLIIYEVRIRASGVSNRFSGSKSGNTASLTISG
LQAEDEADYYCNSYSIHSPWVFGGGTKLTVLRQPKAAPSVTLFPPSSEEL
QANKATLVCLISDFYPGAVTVAWKADSSPVKAGVETTTPSKQSNNKYAAS
SYLSLTPEQWESHRSYSCQVTHEGSTVEKTVAPTECS*
(8.9F V_{L})
SEQ ID NO: 58
METDTLLLWVLLLWVPGSTGDQAGLTQPASVSGSPGQSITISCTAANSDI
GDFNFVSWYQQRPDKAPKLMVYEVSSRPSGVSNRFSGSKSGNTASLTISG
LQAEDEADYYCTSYTSSSTFVFGTGTKVTVLGQPKANPTVTLFPPSSEEL
QANKATLVCLISDFYPGAVTVAWKADSSPVKAGVETTTPSKQSNNKYAAS
SYLSLTPEQWKSHRSYSCQVTHEGSTVEKTVAPTECS*
(NE13 V_{L})
SEQ ID NO: 59
TMETDTLLLWVLLLWVPGSTGDETTLTQSPGTLSLSPGERATLSCRASQS
VSSTYLAWYQQKPGQSPRLLIYGASSRATGIPDRFSGSGSGTQFTLTINR
LEPEDFAVYYCQQFGSPWTFGQGTKVEIKRTVAAPSVFIFPPSDEQLKSG
TASVVCLLNNEYPREAKVQWKVDNALQSGNSQESVTEQDSKDSTYSLSST
LTLSKADYEKHKVYACEVTHQGLSSPVTKSFNRGEC*
(12.1F V_{L})
SEQ ID NO: 60
METDTLLLWVLLLWVPGSTGDETTLTQSPATLSLSPGERATLSCRASQSV
SSYLAWYQHKPGQAPRLLIYGASKRATGIPSRFSGSGSGTDFSLTISSLE
PEDFAVYYCQHRSDWRTTFGQGTRLEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFKRGEC*
(9.8A V_{L})
SEQ ID NO: 61
METDTLLLWVLLLWVPGSTGDDIVMTQSPSTLSASVGDRVTITCRASQSI
DRWLAWYQQKPGKAPKLLIYQASSLERGVPSRFSGSGSGTEFTLTISSLQ
PDDFATYYCQQYNGYPLTFGGGTKVEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSEFRGEC*
(18.5C V_{L})
SEQ ID NO: 62
METDTLLLWVLLLWVPGSTGDDIQMTQSPGTLSLSPGERATLSCRASQSV
ISYYVAWYQHKGGQAPRLLIYGASSRATGVPDRFSGSGSGTDFTLTISSL
EPEDFALYYCQYYGSSPLWAFGQGTKVEIKRTVAAPSVFIFPPSDEQLKS
GTASVVCLLNNFYPREAKVQWKVDNALQSGNSQESV7EQDSKD5TYSLSS
TLTLSKADYEKHKVYACEVTHQGLSSPVTKSFNRGEC*
(8.11G V_{L})
SEQ ID NO: 63
METDTLLLWVLLLWVPGSTGDEIVLTQSPATLSVSPGGRASLSCRASQSI
GDKLSWYQQKPGQAPRLVIYGAYTRATDISPRFSGSRSGTDFNLTISRMQ
SGDFAVYFCQQYENWPRTFGQGTKLEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGEC*
(25.10C V_{L})
SEQ ID NO: 64
METDTLLLWVLLLWVPGSTGDDIQMTQSPSSLSASVGDRVIITCRASQSI
SSSLNWYQQKPGKAPKLLIYAAVNLETGVPSRFSGSGFGTDFTLAISNVQ
PEDFATTYCQQSDTRTFGRGTKLDVKRTVAAPSVFIFPPSDEQLKSGTAS
VVCLLNNFYPREAKVQWKVDNAIQSGNSQESVTEQDSKDSTYSLSSTLTL
SKADYEKHKVYACEVTHQGLSSPVIKSENRGEVLGGRKLGBEGPTCLLQL
TMVTNKAIASQISQIKHFFHCILVVVCPNSSMYLIMSGSGI

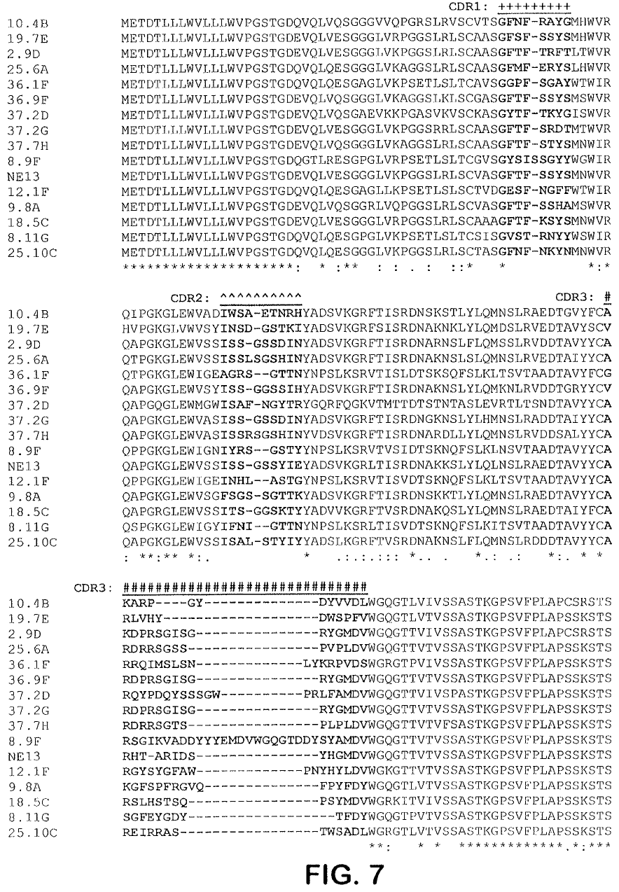

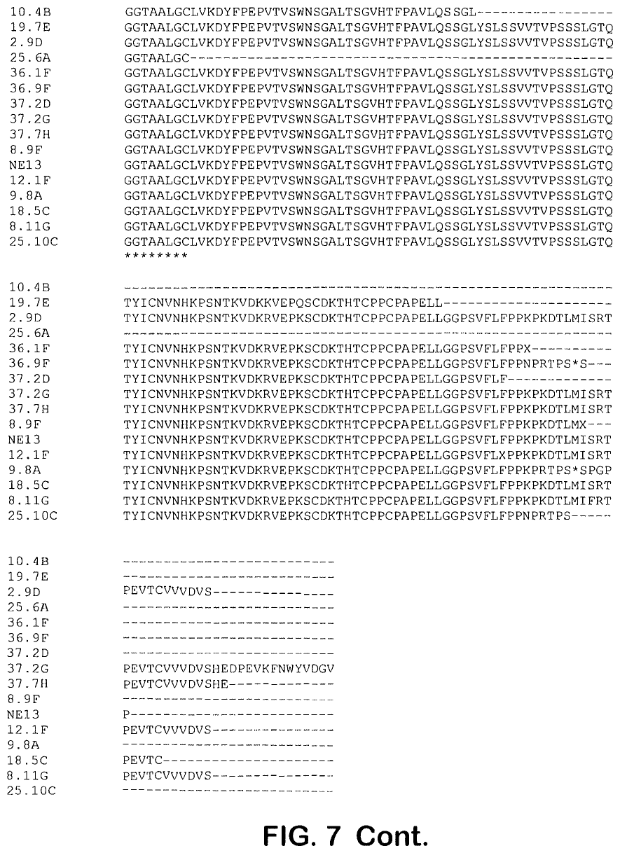

FIG. 7 provides a sequence alignment prepared using CLUSTAL OMEGA™ (1.2.4) multiple sequence alignment (from EMBL-EBI, a part of the European Molecular Biology Laboratory) for the heavy chain variable region amino acid sequences, with CDRs highlighted in bold typeface: CDR1 (marked with +), CDR 2 (marked with \hat{ }), and CDR3 (marked with #).

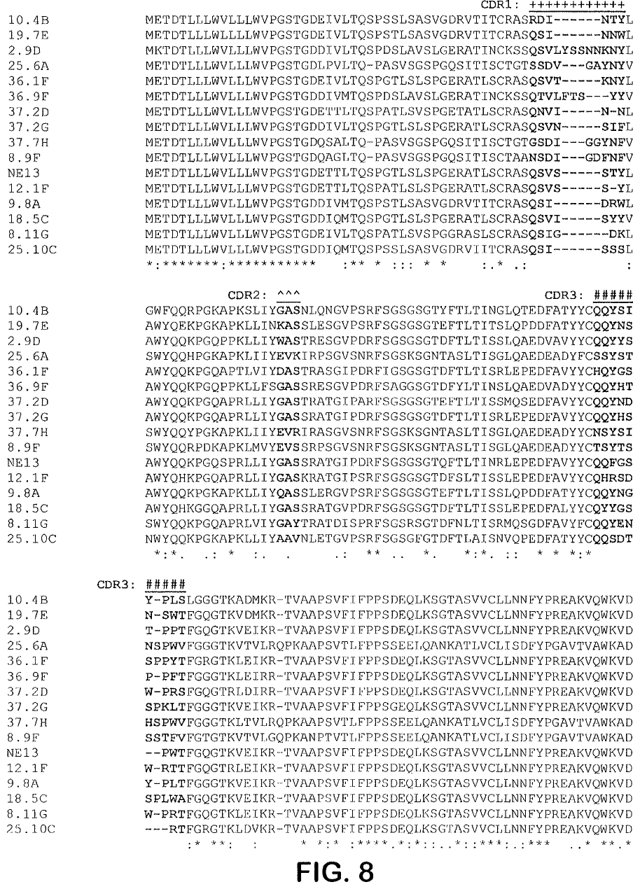

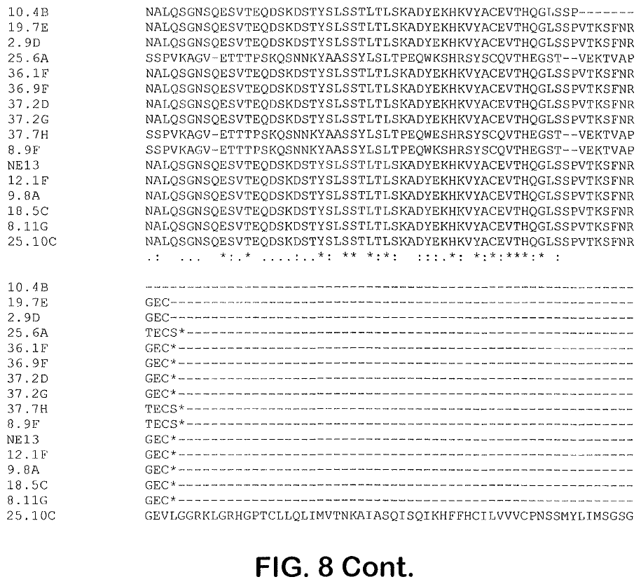

FIG. 8 provides a sequence alignment prepared using using CLUSTAL OMEGA™ (1.2.4) multiple sequence alignment for the light chain variable region amino acid sequences, with CDRs highlighted in bold typeface: CDR1 (marked with +), CDR 2 (marked with \hat{ }), and CDR3 (marked with #).

The HC CDR Sequence Table below lists the sequences of CDR1, CDR 2, and CDR3 of the V_{H} of each of the 16 neutralizing antibodies described herein. The LC CDR Sequence Table below lists the sequences of CDR1, CDR 2, and CDR3 of the V_{L} of each of the 16 neutralizing antibodies described herein.

HC CDR Sequence Table.

| Antibody | HC CDR1 | HC CDR2 | HC CDR3 |
|---|---|---|---|
| 10.4B | GFNFRAYG | IWSAETNRH | AKARPGYDYVVDL |
| | (SEQ ID NO: 65) | (SEQ ID NO: 66) | (SEQ ID NO: 67) |
| 19.7E | GFSFSSYS | INSDGSTKI | VRLVHYDWSPFV |
| | (SEQ ID NO: 68) | (SEQ ID NO: 69) | (SEQ ID NO: 70) |
| 2.9D | GFTFTRFT | ISSGSSDIN | AKDPRSGISGRYGMDV |
| | (SEQ ID NO: 71) | (SEQ ID NO: 72) | (SEQ ID NO: 73) |
| 25.6A | GFMFERYS | ISSLSGSHIN | ARDRRSGSSPVPLDV |
| | (SEQ ID NO: 74) | (SEQ ID NO: 75) | (SEQ ID NO: 76) |
| 36.1F | GGPFSGAY | AGRSGTTN | GRRQIMSLSNLYKRPVDS |
| | (SEQ ID NO: 77) | (SEQ ID NO: 78) | (SEQ ID NO: 79) |
| 36.9F | GFTFSSYS | ISSGGSSIH | VRDPRSGISGRYGMDV |
| | (SEQ ID NO: 80) | (SEQ ID NO: 81) | (SEQ ID NO: 82) |
| 37.2D | GYTFTKYG | ISAFNGYTR | ARQYPDQYSSSGWPRLFAMDV |
| | (SEQ ID NO: 83) | (SEQ ID NO: 84) | (SEQ ID NO: 85) |
| 37.2G | GFTFSRDT | ISSGSSDIN | ARDPRSGISGRYGMDV |
| | (SEQ ID NO: 86) | (SEQ ID NO: 87) | (SEQ ID NO: 88) |
| 37.7H | GFTFSTYS | ISSRSGSHIN | ARDRRSGTSPLPLDV |
| | (SEQ ID NO: 89) | (SEQ ID NO: 90) | (SEQ ID NO: 91) |
| 8.9F | GYSISSGYY | IYRSGSTY | ARSGIKVADDYYYEMD |
| | (SEQ ID NO: 92) | (SEQ ID NO: 93) | VWGQGTDDYSYAMDV |
| | | | (SEQ ID NO: 94) |
| NE13 | GFTFSSYS | ISSGSSYIE | ARHTARIDSYHGMDV |
| | (SEQ ID NO: 95) | (SEQ ID NO: 96) | (SEQ ID NO: 97) |
| 12.1F | GESFNGFF | INHLASTG | ARGYSYGFAWPNYHYLDV |
| | (SEQ ID NO: 98) | (SEQ ID NO: 99) | (SEQ ID NO: 100) |
| 9.8A | GFTFSSHA | FSGSSGTTK | AKGFSPFRGVQFPYFDY |
| | (SEQ ID NO: 101) | (SEQ ID NO: 102) | (SEQ ID NO: 103) |
| 18.5C | GFTFKSYS | ITSGGSKTY | ARSLHSTSQPSYMDV |
| | (SEQ ID NO: 104) | (SEQ ID NO: 105) | (SEQ ID NO: 106) |
| 8.11G | GVSTRNYY | IFNIGTTN | ASGFEYGDYTFDY |
| | (SEQ ID NO: 107) | (SEQ ID NO: 108) | (SEQ ID NO: 109) |
| 25.10C | GFNFNKYN | ISALSTYIY | AREIRRASTWSADL |
| | (SEQ ID NO: 110) | (SEQ ID NO: 111) | (SEQ ID NO: 112) |

LC CDR Sequence Table.

| Antibody | LC CDR1 | LC CDR2 | LC CDR3 |
|---|---|---|---|
| 10.4B | RDINTY | GAS | QQYSIYPLS |
| | (SEQ ID NO: 113) | | (SEQ ID NO: 114) |
| 19.7E | QSINNW | KAS | QQYNSNSWT |
| | (SEQ ID NO: 115) | | (SEQ ID NO: 116) |
| 2.9D | QSVLYSSNNKNY | WAS | QQYYSTPPT |
| | (SEQ ID NO: 117) | | (SEQ ID NO: 118 |
| 25.6A | SSDVGAYNY | EVK | SSYSTNSPWV |
| | (SEQ ID NO: 119) | | (SEQ ID NO: 120) |
| 36.1F | QSVTKNY | DAS | HQYGSSPPYT |
| | (SEQ ID NO: 121) | | (SEQ ID NO: 122) |
| 36.9F | QTVLFTSYY | GAS | QQYHTPPFT |
| | (SEQ ID NO: 123) | | (SEQ ID NO: 124) |
| 37.2D | QNVINN | GAS | QQYNDWPRS |
| | (SEQ ID NO: 125) | | (SEQ ID NO: 126) |
| 37.2G | QSVNSIF | GAS | QQYHSSPKLT |
| | (SEQ ID NO: 127 | | (SEQ ID NO: 128) |
| 37.7H | GSDIGGYNF | EVR | NSYSIHSPWV |
| | (SEQ ID NO: 129) | | (SEQ ID NO: 130) |
| 8.9F | NSDIGDFNF | EVS | TSYTSSSTFV |
| | (SEQ ID NO: 131) | | (SEQ ID NO: 132) |
| NE13 | QSVSSTY | GAS | QQFGSPWT |
| | (SEQ ID NO: 133) | | (SEQ ID NO: 134) |
| 12.1F | QSVSSY | GAS | QHRSDWRTT |
| | (SEQ ID NO: 135) | | (SEQ ID NO: 136) |
| 9.8A | QSIDRW | QAS | QQYNGYPLT |
| | (SEQ ID NO: 137) | | (SEQ ID NO: 138) |
| 18.5C | QSVISYY | GAS | QYYGSSPLWA |
| | (SEQ ID NO: 139) | | (SEQ ID NO: 140) |
| 8.11G | QSIGDK | GAY | QQYENWPRT |
| | (SEQ ID NO: 141) | | (SEQ ID NO: 142) |
| 25.10C | QSISSS | AAV | QQSDTRT |
| | (SEQ ID NO: 143) | | (SEQ ID NO: 144) |

### Diagnostics

The antibodies described herein may be used in a variety of immunoassays for LASV, LCMV, and other arenaviruses. The antibodies of the invention can be produced with high quality control and are suitable as reagents for the purposes of detecting antigen in biological samples. By way of example and not limitation, antibodies of the invention could be used as reagents in an ELISA assay to detect Lassa antigen in a biological sample from a subject. The antibodies can be labeled, e.g., bound to a detectable labelling group such as a fluorescent dye, a quantum dot label, R-phycoerythrin, streptavidin, biotin, an enzyme, a radioisotope, and the like. Such labelling techniques are well known in the antibody art.

### Vaccines

Vaccines for LASV, LCMV, and other arenaviruses also are described herein. In one aspect the vaccines are DNA-based vaccines. One skilled in the art is familiar with administration of expression vectors to obtain expression of an exogenous protein in vivo. See, e.g., U.S. Pat. Nos. 6,436,908; 6,413,942; and 6,376,471. Viral-based vectors for delivery of a desired polynucleotide and expression in a desired cell are well known in the art and non-limiting examples are described herein.

Administration of expression vectors includes local or systemic administration, including injection, oral administration, particle gun or catheterized administration, and topical administration. Targeted delivery of therapeutic compositions containing an expression vector or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Non-viral delivery vehicles and methods can also be employed, including but not limited to, polycationic condensed DNA linked or unlinked to killed adenovirus alone (see, e.g., Cunel, Hum. Gene Ther. (1992) 3:147); ligand-linked DNA (see, e.g., Wu, J. Biol. Chem. (1989) 264:16985); eukaryotic cell delivery vehicles (see, e.g., U.S. Pat. No. 5,814,482; PCT Publication Nos. WO 95/07994; WO 96/17072; WO 95/30763; and WO 97/42338); and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796, WO 94/23697, WO 9 1/14445; and EP 0524968. Additional approaches are described in Philip, Mol. Cell Biol. (1994) 14:2411, and in Woffendin, Proc. Natl. Acad. Sci. (1994) 91:1581.

For human administration, the codons comprising the polynucleotide encoding one or more antibodies specific for LASV glycoprotein and/or LCMV glycoprotein may be optimized for human use, a process that is standard in the art.

In another aspect, one or more antibodies specific to LASV and/or LCMV or combinations thereof is used as a vaccine. The one or more antibodies or combination thereof may be administered by itself or in combination with an adjuvant. Examples of adjuvants include, but are not limited to, aluminum salts, water-in-soil emulsions, oil-in-water emulsions, saponin, QuilA and derivatives, iscoms, liposomes, cytokines including gamma-interferon or interleukin 12, DNA (e.g. unmethylated poly-CpG), microencapsulation in a solid or semi-solid particle, Freunds complete and incomplete adjuvant or active ingredients thereof including muramyl dipeptide and analogues, DEAE dextrarilmineral oil, Alhydrogel, Auspharm adjuvant, and Algammulin.

The antibody vaccine comprising one or more antibodies specific to LASV and/or LCMV or combinations thereof can be administered orally or by any parenteral route such as intravenously, subcutaneously, intraarterially, intramuscularly, intracardially, intraspinally, intrathoracically, intraperitoneally, intraventricularly, sublingually, and/or transdermally.

Dosage and schedule of administration can be determined by methods known in the art. Efficacy of the one or more antibodies specific to LASV and/or LCMV or combinations thereof as a vaccine for Lassa virus, lymphocytic choriomeningitis virus, or related arenaviruses may also be evaluated by methods known in the art.

### Pharmaceutical Compositions

The polynucleotides, polypeptides, and antibodies described herein can further comprise pharmaceutically acceptable carriers, excipients, or stabilizers known in the art (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are non-toxic to recipients at the employed dosages and concentrations, and may comprise buffers such as phosphate, citrate, and other organic acids; antioxidants including ascorbic acid and methionine; preservatives (e.g. octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol); low molecular weight (less than about 10 residues) polypeptides; proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, marmose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

The compositions used in the methods described herein generally comprise, by way of example and not limitation, an effective amount of a polynucleotide or polypeptide (e.g., an amount sufficient to induce an immune response) of the invention or antibody of the invention (e.g., an amount of a neutralizing antibody sufficient to mitigate infection, alleviate a symptom of infection and/or prevent infection).

The pharmaceutical composition can further comprise additional agents that serve to enhance and/or complement the desired effect. By way of example, to enhance the efficacy of the one or more antibodies specific to LASV and/or LCMV or combinations thereof administered as a pharmaceutical composition, the pharmaceutical composition may further comprise an adjuvant. Examples of adjuvants are provided herein.

Also by way of example and not limitation, if the one or more antibodies specific to LASV and/or LCMV or combinations thereof of the invention is being administered to augment the immune response in a subject infected with or suspected of being infected with LASV or LCMV and/or if antibodies of the present invention are being administered as a form of passive immunotherapy, the composition can further comprise other therapeutic agents (e.g., anti-viral agents).

### Kits

Kits for use in the instant methods also are described. Kits include one or more containers comprising by way of example, and not limitation, polynucleotides encoding one or more antibodies specific to LASV and/or LCMV or combinations thereof or fragments thereof of the invention and instructions for use in accordance with any of the methods of the invention described herein. In some embodiments of the kit, the antibodies are bound to a detectable label as discussed above.

Generally, instructions comprise a description of administration or instructions for performance of an assay. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (e.g. the container may be an intravenous solution bag or a vial having a stopper pierceable by a hypodermic injection needle). The container may also have a sterile access port (e.g. the container may be an intravenous solution bag or a vial having a stopper pierceable by a hypodermic injection needle). Kits may optionally provide additional components such as buffers and interpretive information. Normally, the kit comprises a container and a label or package insert(s) on or associated with the container.

The following non-limiting examples are provided to illustrate certain aspects and features of the materials and methods described herein.

### EXAMPLES

### Example 1: LCMV Infection of the Mouse and Recombinant Arenaviruses are a Powerful Experimental System to Assess the Potency and Breath of LCMV Neutralizing mMAbs In Vivo

Both host and viral factors, as well as route of infection and dose of virus influence the outcome of LCMV infection of the mouse. Thus, intravenous (i.v.) inoculation of adult immune competent mice with LCMV Armstrong (ARM) strain results in an acute infection that induces a protective immune response that mediates virus clearance in 10 to 14 days, a process predominantly mediated by virus-specific CD8+ cytotoxic T lymphocytes (CTL). In contrast, i.v. inoculation with a high dose of the immunosuppressive clone 13 (Cl-13) strain of LCMV causes a persistent infection associated with sustained viremia and generalized immune suppression that can last for 60 to 100 days. This model is robust and has clear outcomes, which provide a valid and cost effective experimental system for initial evaluation of the efficacy of antibody-based strategies to control and clear a LCMV infection. In this regard, the use of Cl-13 based recombinant viruses expressing GPs of interest allows assessment of the safety and in vivo neutralizing activity of GP-specific BNmMAbs. This approach is feasible using state-of-the-art arenavirus reverse genetics that allows rescue of infectious recombinant LCM viruses with predetermined mutations of interest, as well as expressing heterologous either viral or non-viral genes of interest. A single-cycle infectious, reporter expressing, recombinant LCMV in which the GP ORF is replaced by GFP (rLCMVΔGP/GFP) was generated. Genetic complementation with plasmids or stable cell lines expressing arenavirus GPs of interest produces the corresponding GP-pseudotyped rLCMVΔGP/GFP that can be used to evaluate antibody responses to HF arenaviruses using a BSL2 platform.

### Example 2: Identification of LASV (Josiah Strain) GP-Specific hMAbs that Cross-React with the GP of LCMV ARM Strain

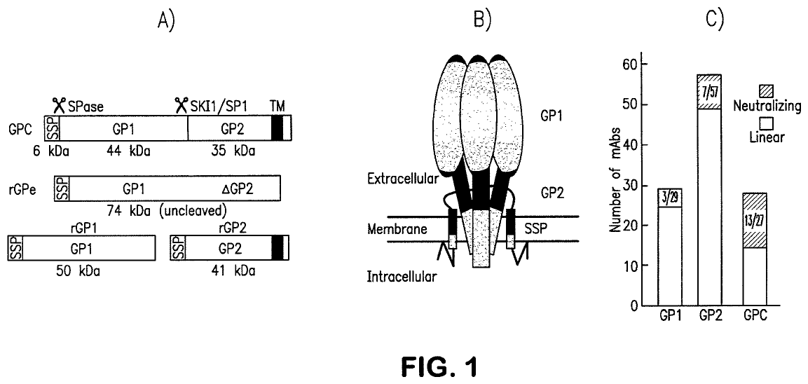

Generation of LASV GP-specific hMAbs: Peripheral blood mononuclear cells (PBMCs) isolated from 17 different LF survivors in Sierra Leone and Nigeria were used to identify B cell clones producing specific IgG to LASV GP. RNA from these B cell clones was used to clone the light chain (LC) and heavy chain (HC) genes. Paired LC and HC were expressed in human 293T cells to generate a collection of 120 LASV GP-specific hMAbs. These hMAbs arose from different germline genes and were likely independently derived. All but one (8.9F) of the hMAbs reacted in ELISA with GP from Josiah strain of LASV (lineage IV), which is closely related to the currently circulating LASV strains in Sierra Leone. LASV GP consists of a SSP and GP1 and GP2 subunits, as shown in FIG. 1, Panels A and B. To define the GP subunits recognized by the LASV hMAbs, an immunofluorescence assay was used to test the recognition by hMAbs of human 293T cells expressing either rGP1 or rGP2 alone, or full-length GP, shown in FIG. 1, Panel C. Twenty-nine hMAbs, including three with neutralizing activity, reacted with LASV rGP1, shown by FIG. 1, Panel C, left and Table 1. Fifty-seven hMAbs recognized LASV rGP2 but none of these exhibited neutralizing activity, as shown by FIG. 1, Panel C, middle and Table 1. Seven hMAbs reacted with peptides representing three linear epitopes in GP2, whereas the remaining hMAbs appeared to recognize conformational epitopes. Twenty-seven hMAbs reacted with cells expressing full-length GP but did not react with either rGP1 or rGP2 expressed individually. Remarkably, thirteen of these hMAbs were neutralizing, as shown by FIG. 1, Panel C, right and Table 1. Inhibitory concentration 50 (IC50) and 80 (IC80) neutralizing activity of LASV GP-specific hMAbs was evaluated using lentivirus particles pseudotyped with the different lineage I-IV LASV GPs. Results are shown in Table 1. Based on the data in Table 1, the antibodies can be classified as most potent (hMAbs exhibiting IC values of <1 μg/mL); potent (hMAbs exhibiting IC values in the range of 1 to 2.5 μg/mL), weak (hMAbs exhibiting IC values of >3 and <20 μg/mL); non-neutralizing (hMAbs exhibiting IC values >20 μg/mL).

TABLE 1
Neutralizing activity of LASV GP-specific hMAbs against LASV lineages I-IV.

| | LASV Josiah (IV) | | LASV 237 (III) | | LASV A19 (II) | | LASV Pinneo(I) | |
|---|---|---|---|---|---|---|---|---|
| Mab | IC50 | IC80 | IC50 | IC80 | IC50 | IC80 | IC50 | IC80 |
| 25.10C | 0.094 | 0.174 | 0.058 | 0.180 | 0.104 | 0.364 | 0.226 | 0.564 |
| 12.1F | 0.158 | 0.562 | 0.146 | 0.458 | 0.463 | 2.266 | 0.285 | 0.692 |
| 8.9F | 0.126 | 1.604 | 0.182 | 3.097 | 0.125 | 0.467 | 0.403 | 2.210 |
| 37.2D | 0.559 | 1.983 | 0.256 | 0.844 | 0.469 | 1.154 | 0.537 | 1.861 |
| 37.7H | 0.191 | 0.532 | 0.077 | 0.202 | 0.255 | 0.537 | 0.301 | 1.658 |
| 25.6A | 0.743 | 1.999 | 0.169 | 0.603 | 0.483 | 3.509 | 1.826 | 3.114 |
| 9.8A | 1.309 | 2.423 | 0.193 | 0.494 | 0.150 | 0.527 | 1.003 | 2.587 |
| 18.5C | 1.935 | 3.985 | 0.621 | 3.231 | 1.200 | 4.633 | 6.111 | 12.170 |
| 8.11G | 0.361 | 1.736 | 1.166 | 3.637 | 1.481 | 4.591 | 3.245 | 10.540 |
| 37.2G | 5.599 | 16.000 | 2.020 | 5.231 | 1.100 | 10.000 | >20 | >20 |
| 2.9D | 6.895 | 16.700 | 1.582 | 5.511 | 3.072 | 14.780 | 10.130 | >20 |
| NE13 | 10.680 | 19.500 | 2.136 | 7.000 | 5.409 | 13.180 | >20 | >20 |
| 19.7E | 5.908 | >20 | 1.062 | 15.000 | >20 | >20 | 1.558 | 3.273 |
| 36.9F | 18.000 | >20 | 4.687 | 19.350 | 13.000 | >20 | 6.984 | >20 |
| 36.1F | 0.248 | 0.755 | >20 | >20 | >20 | >20 | >20 | >20 |
| 10.4B | >20 | >20 | >20 | >20 | >20 | >20 | >20 | >20 |

Neutralizing properties of LASV GP-specific hMAbs: The neutralizing properties of the LASV GP-specific hMAbs were evaluated using envelope-deficient core HIV-1 pseudotyped with LASV GP (LASVpp) (shown in Table 1) and standard plaque reduction neutralization test (PRNT) with authentic LASV. Fifteen of the 120 hMAbs neutralized LASVpp expressing GP from Josiah strain of LASV lineage IV, as shown in Table 1. These neutralizing GP-specific hMAbs were also tested against LASVpp containing GP of the three other LASV lineages I-III (shown in Table 1). The IC50 and IC80 values showed that those with the greatest potency and breadth against all four LASV linages were 25.10C, 12.1F, 8.9F, 37.2D, 37.7H, 25.6A and 8.11G (Table 1). The remaining hMAbs showed weaker and variable potency. Neutralization activity of these GP-specific hMAbs was further confirmed for LASV Josiah strain using a LCMV-based pseudovirus assay. These results revealed that out of the 120 tested LASV GP-specific hMAbs, 15 neutralized to different degrees LASV Josiah strain, and some of them exhibited broad neutralizing activity against representative strains from LASV lineages I-II.

Cross-reactivity of LASV GP-specific hMAbs with LCMV ARM: The 16 LASV GP-specific hMAbs with neutralizing activity (as shown in Table 1) were characterized with respect their ability to recognize LCMV ARM strain GP expressed in human 293T cells transfected with GP-expressing plasmids by immunofluorescence. Human 293T cells transfected with LASV GPs from linages I-IV were included as controls. Nine of the LASV GP-specific neutralizing hMAbs (12.1F, 37.7H, 37.2D, 25.6A, 9.8A, 18.5C, 37.2G, 2.9D and 36.9F) cross-reacted with LCMV ARM GP.

### Example 3: Identification of LASV GP-Specific hMAbs with Broad Cross-Reactivity Against GPs from Different LCMV Strains

The ability of LASV GP-specific neutralizing hMAbs (as shown by FIG. 1 and Table 1) to recognize GPs from five LCMV strains associated with human cases of LCMV-induced disease was examined. These strains corresponded to the WE strain that caused a zoonotic infection in New York in 1935; Rhode Island (RI) strain, responsible for four human cases and three fatalities from a transplant case in 2005; Ohio (OH) strain that is similar to the Michigan LCMV strain responsible for a human case in 2005; Wisconsin (WI) strain responsible for four human deaths in 2003; and Massachusetts (MA) strain, responsible for two human deaths in 2008. LASV GP-specific neutralizing hMAbs 12.1F, 37.2D, 9.8A, 18.5C and 36.9F recognized all five LCMV GP strains. LASV GP-specific neutralizing hMAbs 37.7H, 25.6A, 37.2G and 2.9D recognized four LCMV strains (ARM, WE, WI, and MA, but not RI or AH). The rest of the hMAbs did not cross-react with any of the LCMV strains tested.

### Example 4: Identification of LASV GP-Specific hMAbs with Strong Broadly Neutralizing Activity (BNhMAbs) Against GPs from Different LCMV Strains in Cell-Based Assays

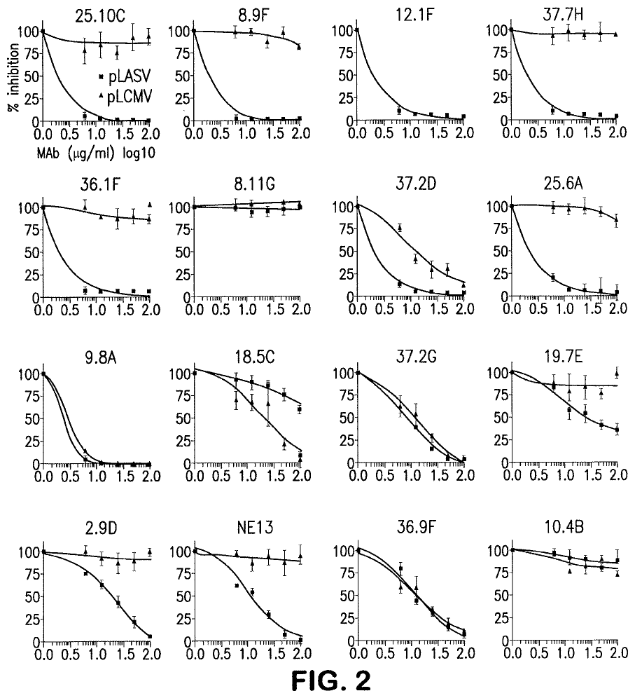

A validated cell-based microneutralization assay was used to identify LASV GP-specific hMAbs that not only cross-reacted with different LCMV GPs, but also neutralized LCMV ARM, as they would represent primary candidates to display broadly antiviral activity in vivo against LCMV strains previously associated with disease cases in humans. From the 15 LASV GP-specific neutralizing hMAbs, six of them (12.1F, 37.2D, 9.8A, 18.5C, 37.2G and 36.9F) neutralized LCMV ARM, as shown in FIG. 2, with IC50<1 μg/mL, with the exception of 18.5C that exhibited a higher (>10 μg/mL) IC50. Results are displayed in Table 2, which shows the neutralizing activity of the 15 LASV GP-specific neutralizing hMAbs against LCMV ARM, and in particular, the IC50 and IC80 values of the 15 LASV GP-specific neutralizing hMAbs against LCMV ARM. Values were obtained from the cell-based microneutralization assay (shown in FIG. 2) using LASV or LCMV GP-pseudotyped rLCMVΔGP/GFP viruses. Grey indicates LASV GP-specific neutralizing hMAbs that neutralized LCMV GP ARM. Neutralization of LASV GP-pseudotyped rLCMVΔGP/GFP was similar to neutralization results obtained using the LASV GP-pseudotyped lentivirus particles shown in Table 1.

TABLE 2
Neutralizing activity of 15 LASV GP-specific neutralizing
hMAbs against LCMV Armstrong strain (ARM).

| | LASV Josiah (IV) | | LCMV ARM | |
|---|---|---|---|---|
| hMAb | IC50 | IC80 | IC50 | IC80 |
| 25.10C | 0.160 | 0.247 | >10 | >10 |
| 12.1F | 0.172 | 0.258 | 0.167 | 0.265 |
| 8.9F | 0.134 | >10 | >10 | >10 |
| 37.2D | 0.137 | 0.260 | 0.518 | 2.358 |
| 37.7H | 0.134 | 0.214 | >10 | >10 |
| 25.6A | 0.188 | 0.300 | >10 | >10 |
| 9.8A | 0.139 | 0.253 | 0.112 | 0.248 |
| 18.5C | >10 | >10 | 2.207 | 4.83 |
| 8.11G | >10 | >10 | >10 | >10 |
| 37.2G | 0.405 | 1.776 | 0.525 | 2.461 |
| 2.9D | 0.942 | 2.706 | >10 | >10 |
| NE13 | 0.567 | 1.763 | >10 | >10 |
| 19.7E | 1.189 | >10 | >10 | >10 |
| 36.9F | 0.570 | 2.228 | 0.591 | 3.328 |
| 36.1F | 0.132 | 0.206 | >10 | >10 |
| 10.4B | >10 | >10 | >10 | >10 |

### Example 5: In Vivo Characterization of Selected GP-Specific BNhMAb

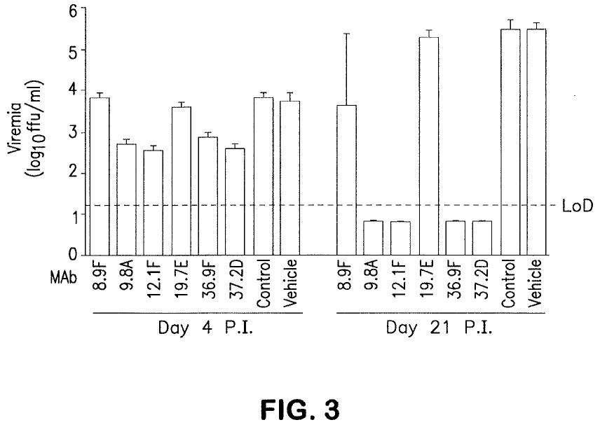

The well-characterized mouse model of LCMV infection was used to test whether LASV GP-specific neutralizing hMAbs with broadly neutralizing activity against LCMV (shown in FIG. 2 and Table 2) also exhibited in vivo neutralizing activity. The immunosuppressive Clone 13 (Cl-13) strain of LCMV was used. Infection (i.v.) of B6 WT mice with a high dose (≥10⁶ PFU) of Cl-13 results in transient generalized immunosuppression and establishment of a persistent infection with well-established parameters. Virus clearance takes place between days 60 to 100 (post inoculation (p.i.). However, treatment of Cl-13 infected mice that results in reduced viral load accelerates Cl-13 clearance. Therefore, it was predicted that LASV GP-specific neutralizing hMAbs exhibiting in vivo neutralizing activity would either prevent the establishment of Cl-13 persistence or accelerate its clearance. Mice were treated with the indicated hMAbs at 20 mg/Kg intraperitoneally (i.p.) and were infected with either rCl-13/WT or rCl-13/LASV-GP(mCD). rCl-13/LASV-GP(mCD) was used because it contains mutations C459K and K461G within the cytosolic domain of GP that enhance persistence in mice. The in vivo results are shown in FIG. 3 and correlate with those previously documented in cultured cells (shown in FIG. 2 and Table 2). Mice treated with hMAbs 12.1F, 37.2D, 9.8A and 36.9F prevented persistence of rCl-13/WT. Unexpectedly, hMAbs 37.2G and 18.5C did not prevent Cl-13 persistence in vivo. As expected, based on cross-reactivity and neutralization results in cultured cells, hMAbs 19.7E and 8.9F did not prevent persistence of rCl-13.

Table 3 displays a summary of the cross-reactivity and neutralizing activity in vitro and in vivo of LASV GP-specific hMAbs against six LCMV strains (ARM, WE, RI, OH, WI, and MA) tested.

TABLE 3
Summary of the cross-reactivity and neutralizing activity in
vitro and in vivo of LASV GP-specific hMAbs against LCMV.

| | Cross-reactivity | | | | | | Neutralizing activity | |
|---|---|---|---|---|---|---|---|---|
| | LCMV_{ARM} | LCMV_{WE} | LCMV_{RI} | LCMV_{OH} | LCMV_{WI} | LCMV_{MA} | In vitro | In vivo |
| 25.10C | − | − | − | − | − | − | − | − |
| 8.9F | − | − | − | − | − | − | − | − |
| 12.1F | + | + | + | + | + | + | + | + |
| 37.7H | + | + | − | − | + | + | − | − |
| 36.1F | − | − | − | − | − | − | − | − |
| 8.11G | − | − | − | − | − | − | − | − |
| 37.2D | + | + | + | + | + | + | + | + |
| 25.6A | + | + | − | − | + | + | − | − |
| 9.8A | + | + | + | + | + | + | + | + |
| 18.5C | − | − | − | − | − | − | + | − |
| 37.2G | + | + | − | − | + | + | + | − |
| 19.7E | − | − | − | − | − | − | − | − |
| 2.9D | + | + | − | − | + | + | − | − |
| NE13 | − | − | − | − | − | − | − | − |
| 36.9F | + | + | + | + | + | + | + | + |
| 10.4B | − | − | − | − | − | − | − | − |

### Example 6: Assay Development

A panel of murine antibodies against Fab or F(ab′)2 fragments of leading candidate therapeutic BNhMAbs was derived for isolation of highly specific anti-idiotypic reagents for assay development. In order to develop a highly protective therapeutic BNhMAb cocktail containing two to four antibodies that together confer maximum pre- and post-exposure protection against LCMV infections, while minimizing the emergence of escape mutants, it is important to characterize the PK of each antibody when administered in a cocktail form. To distinguish between all BNhMAbs included in the cocktail after administration, highly specific anti-idiotypic antibodies are the best tool available to rapidly determine concentration and clearance of individual hMAbs from the blood. A panel of anti-idiotypic antibodies to 37.2D and 12.1F has been developed. Anti-idiotypic mMAbs to 37.2D have specifically detected this BNhMAb when spiked into human serum. The anti-idiotypic antibodies do not capture or detect any other arenaviral BNhMAb tested or any other IgG specificity present in human serum on both ELISA and SPR based studies, and thus are useful for assaying 37.2D.

### Example 7: Therapeutic Efficacy of First-In-Class Human LASV-Specific Antibodies in Guinea Pig (GP) and Cynomolgus Macaque (CM) Models of Lassa Fever

These studies were done under BSL-4 biocontainment at the Galveston National Laboratory. Outbred Hartley strain GP were challenged i.p. with 1,000 pfu of GP adapted (GPA) LASV Josiah strain (N=5/group). This model has been described recently for testing therapeutics against LASV. The advantage of using outbred animals to model human infection is inferred from the higher variability of immune responses inherent in outbred populations. Viremia was compared by Kruskal-Wallis test supported by Dunn's Multiple comparison posttest (PRISM 5™ software available from GraphPad Software, La Jolla, CA) to detect differences from the control group for time points relevant to onset (day 7) or peak viremia (day 14) as determined from historical data.

Eleven LASV hMAbs tested in a Hartley GP model of LF segregated into three distinct protection groups: (1) 25.6A, 2.9D, 8.9F, 12.1F, and 37.7H conferred 100% protection and no change in clinical score in GPs. (2) 37.2D, 19.7E, and 37.2G protected 80 to 90% of animals. (3) 10.4B, 25.10C, and 36.1F, conferred 40%, 30%, and 20% protection, respectively. An irrelevant recombinant human isotype control (IgG1) Ab did not confer protection (0% survival).

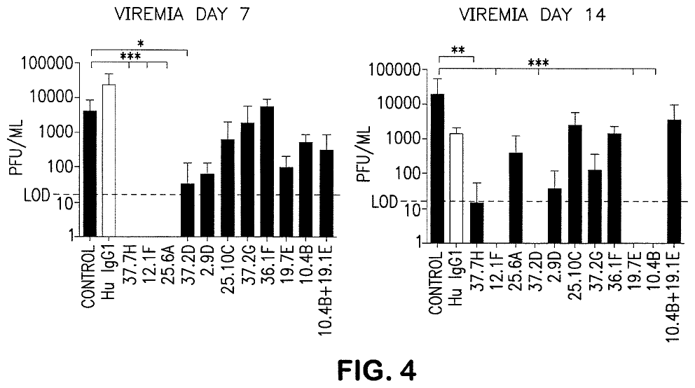

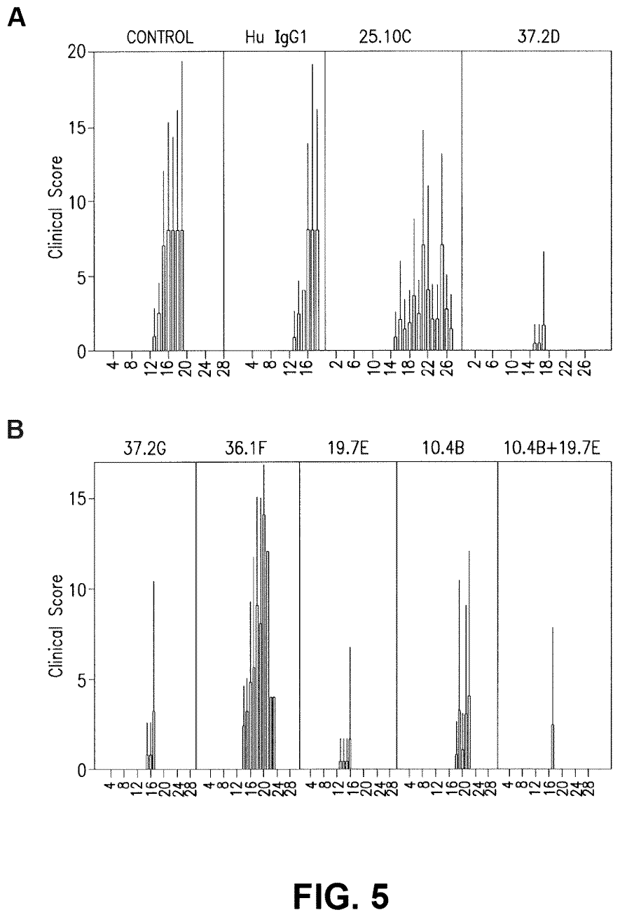

With respect to viremia, untreated control animals averaged 3.5 and 4.5 Log PFU/mL on days 7 and 14, respectively, as shown in FIG. 4. Despite 100% protection at the study endpoint, some animals from treatment group 8.9F or 37.7H, 2.9D, and 25.6A had low level viremia on day 7 or 14, respectively. Treatment groups where 90% protection was afforded (37.2D and 19.7E) had reduced mean viremia titers and minimal clinical score values. Treatment groups with 80% or less survival had comparable mean viremia titers to control animals on day 7, but by day 14 mean viremia was markedly lower than control animals. Groups with 80 to 90% survival exhibited relatively low mean clinical scores (FIG. 5) and all remaining treatment groups exhibited concomitant increases in mean clinical scores with decreases in survival per group. Endpoint viremia was not determined for these studies as survival was the primary metric of interest, though all surviving animals demonstrated no clinical signs.

Results from the guinea pig studies informed studies for the Cynomolgus macaque (CM) model of LF. These studies demonstrated that several of the antibodies with high potency in the GP model also protected 100% of the CMs when administered on the day of challenge. 19.7E protected 75% of CMs. Notably a treatment dose as low as 6 mg/kg of hMAb 37.2D provided 100% protection in CMs, whereas 19.7E protected 75% of CMs. A cocktail of three human MAbs (37.2D, 12.1F, and 8.9F at 15 mg/kg each) rescued 100% of CMs even after delay in the start of treatment to 3, 6, or 8 days post-infection (therapeutic walk-out studies). At 8 days post-infection, untreated CMs had developed high viral loads and were extremely ill. CM also were protected from lethal LF induced by challenge with either strain Josiah (lineage IV) or a contemporary lineage II strain derived from a lethal case of LF in Nigeria, both with the first treatment administered at 8 days post-infection.

### Example 8: Structural Definition of the Anti-LASV 37.7H Epitope

Monomeric GPCysR4 was incubated with excess Fab 37.7H and subjected to SEC-MALS analysis. SEC-MALS indicated the formation of trimeric GP-Fab complexes in addition to monomeric GP-Fab complexes. Crystals of both the monomeric and trimeric fractions of the GPCysR4-Fab 37.7H complex formed in space group P6122 and diffract to 3.2 Å with a trimer of GP bound to three Fabs in the asymmetric unit. Phases were determined with an iterative approach by using molecular replacement with a related Fab structure and the LCMV GP crystal structure.

The antibody 37.7H against LASV neutralizes viruses representing all four known lineages of LASV in vitro and offers protection from lethal LASV challenge in guinea pig and nonhuman primates. The antibody simultaneously binds two GP monomers at the base of the GP trimer, where it engages four discontinuous regions of LASV GP, two in “site A” and two in “site B”. Site A contains residues 62 to 63 of the N-terminal loop of GP1 and residues 387 to 408 in the T-loop and HR2 of GP2. Site B contains residues 269 to 275 of the fusion peptide and residues 324 to 325 of HR1 of GP2. In total, 37.7H buries about 1620 Å² of GP: about 1000 Å² of GP at site A and about 620 Å² of GP at site B. Although nearly the entire surface buried on GP belongs to GP2, the presence of both GP1 and GP2 is critical for 37.7H recognition, likely because GP1 is required to maintain the proper prefusion conformation of GP2 for 37.7H binding.

The antibody 37.7H also recognizes the GPC of LCMV but does not recognize the GPC of the more distantly related Old World arenavirus LUJV nor the GPC of New World arenaviruses. A sequence comparison among these arenaviruses demonstrates nearly complete sequence conservation throughout the 37.7H epitope for all LASV lineages and LCMV. However, the sequences of LUJO, JUNV, and MACV GPCs are far more divergent, particularly in HR2 of GP2, which is heavily involved in binding to 37.7H. The 37.7H antibody neutralizes by stabilizing the prefusion GP.

The quaternary nature and the involvement of the fusion peptide in the 37.7H epitope suggest that this antibody neutralizes the virus by stabilizing GPC in the prefusion conformation, thereby preventing the conformational changes required for infection. This was verified by analyzing the ability of LASV GP-pseudotyped recombinant vesicular stomatitis virus (rVSV-LASV GP) to mediate fusion with cell membranes.

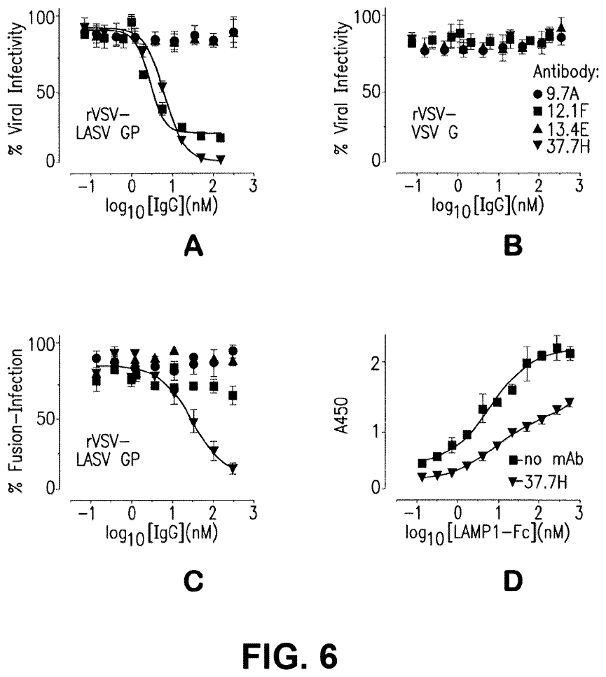

First the ability of 37.7H to neutralize rVSVLASV GP was determined. FIG. 6 shows the effect of antibodies on rVSV-LASV GP infection and fusion. Antibody-mediated neutralization of rVSV-LASV GP is shown in FIG. 6, Panel A. Antibody-mediated neutralization of rVSV-VSV-G is shown in FIG. 6, Panel B. The antibody 9.7A is non-neutralizing antibody and in the same competition group as 37.7H (GPC-B); 13.4E binds to a linear epitope in the T-loop of GP2; 12.1F binds to the GP1 subunit of LASV. Error bars indicate the standard deviation of at least six (two biological replicates, each having three or more technical replicates). FIG. 6, Panel C shows antibody-mediated inhibition of rVSVLASV GP fusion at the cell surface. Error bars indicate the standard error of the mean of six (except 37.7H, where N=9). FIG. 6, Panel D shows Fab 37.7H reduces binding of a LAMP1-Fc fusion protein to LASV GPCysR4. Error bars indicate the standard deviation of six and three technical replicates.

37.7H effectively prevented cellular infection by rVSV-LASV GP, as did the antibody 12.1F, which binds to the upper, 3-sheet face of LASV GP1 and is presumed to block cell attachment. In contrast, antibodies 13.4E, which binds a linear epitope in the T-loop, and 9.7A, which is a non-neutralizing GPC-B antibody, did not prevent viral infection (FIG. 6, Panels A and B).

Next, the ability of 37.7H to prevent fusion of rVSV-LASV GP with cell membranes when exposed to low pH was examined. Unlike the non-neutralizing antibodies 9.7A and 13.4E, which were not effective in preventing fusion, 37.7H reduced fusion by nearly 80% compared with rVSV-LASV GP alone (FIG. 6, Panel C). In contrast, the neutralizing antibody against GP1 (anti-GP1), 12.1F, showed only a slight reduction in infectivity, suggesting that the effect of 37.7H was strictly due to disruption in fusogenicity of the GPC and not attachment to cells.

Before exposure of the GP2 fusion peptide and loop and subsequent fusion of the viral and host cell membranes, LASV GP1 engages LAMP1. Engagement of this receptor is thought to require conformational changes in GP1 that are triggered by exposure to the low pH in the endosome. Tomography of LASV spikes in the presence of low pH and LAMP1 shows an opening of the trimer compared with its neutral pH conformation. To determine whether 37.7H could prevent these conformational changes, the ability of GPCysR4 to bind to a soluble LAMP1-Fc fusion alone and when bound to Fab 37.7H was analyzed. In the absence of Fab 37.7H, GPCysR4 effectively bound to LAMP1 when exposed to low pH. In the presence of Fab 37.7H, however, interaction between GPCysR4 and LAMP1 was markedly reduced (FIG. 6, Panel D).

Based on crystallographic data, the footprint of 37.7H and the footprint of LAMP1 are separated by about 50 Å, and the angle adopted by the bound Fab fragments of 37.7H suggests that it is unlikely to sterically interfere with LAMP1. Thus, there are likely to be conformational changes in GP1 required for LAMP1 binding that are prevented by this human survivor antibody. Taken together, these results demonstrate that the probable mechanism of action for 37.7H and probably for other antibodies in its potent GPC-B competition group is stabilization of the prefusion GPC trimer and prevention of the conformational changes required for binding of LAMP1 and triggering of the GP2 fusion peptide and fusion loop in the endosome.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the claims.

The following reference articles are incorporated herein by reference.

### REFERENCES

1. Auperin, D. D., Sasso, D. R. and McCormick, J. B. (1986). Nucleotide sequence of the glycoprotein gene and intergenic region of the Lassa virus S genome RNA. Virology 154, 155-167.
2. Beyer, W. R., Popplau, D., Garten, W., von Laer, and Lenz O. (2003). Endoproteolytic processing of the lymphocytic choriomeningitis virus glycoprotein by the sibtilase SKI-1/S1P. J. Virol. 77, 2866-2872.
3. Buchmeier, M. J. (2002). Arenaviruses: protein structure and function. Curr. Top. Microbiol. Immunol. 262, 259-173.
4. Buchmeier, M. J., and Parekh, B. S. (1987). Protein structure and expression among arenaviruses. Current Topics in Microbiology and Immunology 133, 41-57.
5. Buchmeier, M. J., Lewicki, H. A., Tomor, O., and Jonhson, K. M. (1980). Monoclonal antibodies to lymphocytic choriomeningitis virus reacts with pathogenic arenaviruses. Nature, London 288, 4876-4877.
6. Burnette, W. N. (1981). “Western Blotting”: electrophoretic transfer of proteins from sodium dodecyl sulfate-polyacrylamide gels to unmodified nitrocellulose and radiographic detection with antibody and radioiodinated protein A. Analytical Biochemistry 112, 195-203.
7. Clegg, J. C. and Lloyd, G. (1983). Structureal and cell-associated proteins of Lassa virus. Journal of General Virology 64, 1127-1136.
8. Eichler, R., Lenz, O., Strecker, T., Eickmann, M., Klenk, H. D., and Garten, W. (2004). Lassa virus glycoprotein signal peptide displays a novel topology with an extended ER-luminal region. J. Biol. Chem. 279, 12293-12299.
9. Eichler, R., Lenz, O., Strecker, T., Eickmann, M., Klenk, H. D., and Garten, W. (2003). Identification of Lassa virus glycoprotein signal peptide as a trans-acting maturation factor. EMBO Rep. 4, 1084-1088.
10. Eichler, R., Lenz, O., Strecker, T., Eickmann, and Garten, W. (2003). Signal peptide of Lassa virus glycoprotein GP-C exhibits an unusual length. FEBS Lett. 538, 203-206.
11. Elagoz, A., Benjannet, S., Mammarbassi, A., Wickham, L., and Seidah, N. G. (2002). Biosynthesis and cellular trafficking of the convertase SKI-1/S1P: ectodomain shedding requires SKI-1 activity. J. Biol. Chem. 277, 11265-11275.
12. Hufert, F. T., Ludke, W., and Schmitz, H. (1989). Epitope mapping of the Lassa virus nucleocapsid protein using monoclonal anti-nucleocapsid antibodies. Archives of Virology 106, 201-212.
13. Lenz, O., ter Meulen, J., Feldmann, H., Lenk, H.-D., and Garten, W. (2000). Identification of a novel consensus sequence at the cleavage site of the Lassa virus glycoprotein. J. Virol. 74, 11418-11421.
14. Lukashevich L. S., Clegg J. C., and Sidibe K. (1993). Lassa virus activity in Guinea: distribution of human antiviral antibody defined using enzyme-linked immunosorbent assay with recombinant antigen. J Med Virol. 40, 210-7.
15. McCormick, J. B., and Fisher-Hoch, S. P. (2002). Lassa Fever. Curr. Top. Microbiol. Immunol. 262, 75-109.
16. Ruo, S. L., Mitchell, S. W., Killey, M. P., Roumillat, L. F., Fisher-Hoch, S. P., and McCormick, J. B. (1991). Antigenic relatedness between arenaviruses defined at the epitope level by monoclonal antibodies. Journal of General Virology 72, 549-555.
17. Sanchez, A., Pifat, D. Y., Kenyon, R. H., Peters, C. J., McCormick, J. B., and Kiley, M. P. (1989). Junin virus monoclonal antibodies: characterization and cross-reactivity with other arenaviruses. J. Gen. Virol. 70, 1125-1132.
18. Spiropoulou, C. F., Kunz, S., Rollin, P. E., Campbell, K. P., and Oldstone, M. B. A. (2002). New World arenavirus clade C, but not clade A and B viruses, utilizes a-dystroglycan as its major receptor. J. Virol. 76, 5140-5146.
19. ter Meulen J., Badusche M., Kuhnt K., Doetze A., Satoguina J., Marti T., Loeliger C., Koulemou K., Koivogui L., Schmitz H., Fleischer B., and Hoerauf A. (2000). Characterization of human CD4(+) T-cell clones recognizing conserved and variable epitopes of the Lassa virus nucleoprotein. J. Virol. 74, 2186-92.
20. ter Meulen, J., Koulemou K., Wittekindt T., Windisch K., Strigl S., Conde S., and Schmitz H. (1998). Detection of Lassa Virus Antinucleoprotein Immunoglobulin G (IgG) and IgM Antibodies by a Simple Recombinant Immunoblot Assay for Field Use. J. Clin. Microbiol. 36, 3143-3148.
21. York, J., Agnihothram, S. S., Ronamowski, V., and Nunberg, J. H. (2005). Genetic analysis of heptad-repeat regions in the G2 fusion subunit of the Junin arenavirus envelope glycoprotein. Virology 343, 267-279.
22. York, J., Ronamowski, V., Lu, M., and Nunberg, J. H. (2004). The signal peptide of the Junin arenavirus envelope glycoprotein is myristoylated and forms an essential subunit of the mature G1-G2 complex. J. Virol. 78, 10783-10792.
23. Shaffer J G, Grant D S, Schieffelin J S, Boisen M L, Goba A, et al. 2014. Lassa Fever in Post-Conflict Sierra Leone. PLoS Negl Trop Dis 8: e2748.
24. Hartnett J N, Boisen M L, Oottamasathien D, Jones A B, Millett M M, . . . Garry R F, Branco L M & the VHFC (2015). Current and emerging strategies for the diagnosis, prevention and treatment of Lassa fever. Future Virology. Review. Vol. 10, No. 5, Pages 559-584.
25. Andersen K G, Shapiro B J, Matranga C B, Sealfon R, Lin A E, . . . Branco L M, Gire S K, Phelan E, Tariyal R, Tewhey R, . . . Garry R F, Sabeti P C. Clinical Sequencing Uncovers Origins and Evolution of Lassa Virus. Cell. 2015 Aug. 13; 162(4):738-50.
26. Luis M Branco, Jessica N Grove, Matt L Boisen, Jeffrey G Shaffer, Augustine Goba, . . . Robert F Garry. Emerging trends in Lassa fever: redefining the role of immunoglobulin M and inflammation in diagnosing acute infection. Virology Journal 2011, 8:478 (24 Oct. 2011).
27. Jessica N Grove, Luis M Branco, Matt L Boisen, Ivana J Muncy, Lee A Henderson, . . . Robert F Garry. Capacity building permitting comprehensive monitoring of a severe case of Lassa hemorrhagic fever in Sierra Leone with a positive outcome: Case Report. Virology Journal 2011, 8:314 (20 Jun. 2011).
28. Luis M Branco, Jessica N Grove, Frederick J Geske, Matt L Boisen, Ivana J Muncy, . . . Robert F Garry. Lassa virus-like particles displaying all major immunological determinants as a vaccine candidate for Lassa hemorrhagic fever. Virology Journal 2010, 7:279 (20 Oct. 2010).
29. Luis M Branco, Matt L Boisen, Kristian G Andersen, Jessica N Grove, Lina M Moses, . . . Robert F Garry. Lassa Hemorrhagic Fever in a Late Term Pregnancy from Northern Sierra Leone with a Positive Maternal Outcome: Case Report. Virology Journal 2011, 8:404 (15 Aug. 2011).

1-27. (canceled)

28. An antigen-binding composition comprising a combination of three recombinant monoclonal neutralizing antibodies or neutralizing antigen-binding antibody fragments thereof, which are specific to Lassa virus glycoprotein, wherein the composition comprises:
a recombinant human monoclonal antibody or an antigen-binding antibody fragment thereof comprising a V_{H} CDR1 of SEQ ID NO: 83, a V_{H} CDR2 of SEQ ID NO: 84, a V_{H} CDR3 of SEQ ID NO: 85, a V_{L} CDR1 of SEQ ID NO: 125, a V_{L} CDR2 of sequence Gly Ala Ser, and a V_{L} CDR3 of SEQ ID NO: 126;
a recombinant human monoclonal antibody or an antigen-binding antibody fragment thereof comprising a V_{H} CDR1 of SEQ ID NO: 92, a V_{H} CDR2 of SEQ ID NO: 93, a V_{H} CDR3 of SEQ ID NO: 94, a V_{L} CDR1 of SEQ ID NO: 131, a V_{L} CDR2 of sequence Glu Val Ser, and a V_{L} CDR3 of SEQ ID NO: 132; and
a recombinant human monoclonal antibody or an antigen-binding antibody fragment thereof comprising a V_{H} CDR1 of SEQ ID NO: 98, a V_{H} CDR2 of SEQ ID NO: 99, a V_{H} CDR3 of SEQ ID NO: 100, a V_{L} CDR1 of SEQ ID NO: 135, a V_{L} CDR2 of sequence Gly Ala Ser, and a V_{L} CDR3 of SEQ ID NO: 136.

29. The composition of claim 28, wherein each antigen-binding antibody fragment is selected from the group consisting of a Fab, a Fab′, and a F(ab′)₂ fragment.

30. A pharmaceutical composition for treating a Lassa virus or a lymphocytic choriomeningitis virus infection comprising the composition of claim 28 and a pharmaceutically acceptable carrier.

31. The composition of claim 28 wherein the composition comprises:
a recombinant human monoclonal antibody or an antigen-binding antibody fragment thereof comprising a V_{H} of SEQ ID NO: 39 and a V_{L} of SEQ ID NO: 55;
a recombinant human monoclonal antibody or an antigen-binding antibody fragment thereof comprising a V_{H} of SEQ ID NO: 42 and a V_{L} of SEQ ID NO: 58; and
a recombinant human monoclonal antibody or an antigen-binding antibody fragment thereof comprising a V_{H} of SEQ ID NO: 44 and a V_{L} of SEQ ID NO: 60.

32. The composition of claim 31, wherein each antigen-binding antibody fragment is selected from the group consisting of a Fab, a Fab′, and a F(ab′)₂ fragment.

33. A pharmaceutical composition for treating infection by a Lassa virus or a lymphocytic choriomeningitis virus comprising the composition of claim 31 and a pharmaceutically acceptable carrier.

34. An antigen-binding composition comprising a recombinant human monoclonal neutralizing antibody or a neutralizing antigen-binding antibody fragment thereof, which is specific for Lassa virus glycoprotein; the antibody or antibody fragment thereof comprising a V_{H} CDR1 of SEQ ID NO: 83, a V_{H} CDR2 of SEQ ID NO: 84, a V_{H} CDR3 of SEQ ID NO: 85, a V_{L} CDR1 of SEQ ID NO: 125, a V_{L} CDR2 of sequence Gly Ala Ser, and a V_{L} CDR3 of SEQ ID NO: 126.

35. The composition of claim 34, wherein the antigen-binding antibody fragment is selected from the group consisting of a Fab, a Fab′, and a F(ab′)₂ fragment.

36. The composition of claim 34 wherein the composition comprises a recombinant human monoclonal antibody or an antigen-binding antibody fragment thereof comprising a V_{H} of SEQ ID NO: 39 and a V_{L} of SEQ ID NO: 55.

37. A pharmaceutical composition for treating infection by a Lassa virus or a lymphocytic choriomeningitis virus comprising the composition of claim 34 and a pharmaceutically acceptable carrier.

38. An antigen-binding composition comprising a recombinant human monoclonal neutralizing antibody or a neutralizing antigen-binding antibody fragment thereof, which is specific for Lassa virus glycoprotein; the antibody or antibody fragment thereof comprising a V_{H} CDR1 of SEQ ID NO: 92, a V_{H} CDR2 of SEQ ID NO: 93, a V_{H} CDR3 of SEQ ID NO: 94, a V_{L} CDR1 of SEQ ID NO: 131, a V_{L} CDR2 of sequence Glu Val Ser, and a V_{L} CDR3 of SEQ ID NO: 132.

39. The composition of claim 38, wherein the antigen-binding antibody fragment is selected from the group consisting of a Fab, a Fab′, and a F(ab′)₂ fragment.

40. The composition of claim 38, wherein the composition comprises a recombinant human monoclonal antibody or an antigen-binding antibody fragment thereof comprising a V_{H} of SEQ ID NO: 42 and a V_{L} of SEQ ID NO: 58.

41. A pharmaceutical composition for treating infection by a Lassa virus or a lymphocytic choriomeningitis virus comprising the composition of claim 38 and a pharmaceutically acceptable carrier.

42. A method of treating or preventing a Lassa virus infection or a lymphocytic choriomeningitis virus infection in a subject comprising administering the composition of claim 28 to the subject.

43. A method of treating or preventing a Lassa virus infection or a lymphocytic choriomeningitis virus infection in a subject comprising administering the composition of claim 31 to the subject.

44. A method of treating or preventing a Lassa virus infection or a lymphocytic choriomeningitis virus infection in a subject comprising administering the composition of claim 34 to the subject.

45. A method of treating or preventing a Lassa virus infection or a lymphocytic choriomeningitis virus infection in a subject comprising administering the composition of claim 36 to the subject.

46. A method of treating or preventing a Lassa virus infection in a subject comprising administering the composition of claim 38 to the subject.

47. A method of treating or preventing a Lassa virus infection in a subject comprising administering the composition of claim 40 to the subject.


SEQUENCE LISTING (144 sequences)

SEQ ID NO: 1 - DNA, 670 bp, synthetic construct
  misc_feature 1..670: note = cDNA 10.4B HC variable region
  source 1..670: mol_type = other DNA
tgcgcgttacngatccaagctgtgaccggcgcctacctgagatcaccggtgctagcaccatggagacagacacactcctgctatgggtactgctgctctgggttccaggttccactggtgaccaggtgcagctggtacagtctgggggaggcgtggtccagcctgggaggtccctgagagtctcctgtgttacgtctggattcaatttcagagcctacggcatgcactgggtccgccagattccaggcaagggactggagtgggtggcagatatttggtctgccgagactaatagacactatgcagattccgtgaagggccgattcaccatctccagagacaactccaagagcacactgtatctgcaaatgaacagcctgagagccgaggacacgggcgtatatttctgtgccaaagcgcgaccaggctatgattatgtcgttgacttatggggccagggaacgctggtcatcgtctcctcagcttccaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactcta

SEQ ID NO: 2 - DNA, 819 bp, synthetic construct
  misc_feature 1..819: note = cDNA 19.7E HC variable region
  source 1..819: mol_type = other DNA
atccagctgtgaccggcgcctacctgagatcaccggtgctagcaccatggagacagacacactcctgctatgggtactgctgctctgggttccaggttccactggtgacgaggtgcagctggtggagtctgggggaggcttagttcggcctggggggtccctgagactctcctgtgcagcctctggattctccttcagtagctactcgatgcactgggtccgccatgttcctgggaaggggctggtgtgggtctcatatattaatagtgatgggagtactaaaatctacgcggactccgtgaagggccgattctccatctccagagacaatgccaagaacaagctctatctgcaaatggacagtttgagagtcgaggacacggctgtatattcgtgtgtaaggcttgtacattacgactggtccccattcgtgtggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagccccaatcttgtgacaaaactcacacatgcccaccgtgcccagcacctgaactcct

SEQ ID NO: 3 - DNA, 920 bp, synthetic construct
  misc_feature 1..920: note = cDNA 2.9D HC variable region
  source 1..920: mol_type = other DNA
gtcactgcacctcggttctatcgattggctagcaccatggagacagacacactcctgctatgggtactgctgctctgggttccaggttccactggtgacgaggtgcagctggtggagtctgggggaggcctggtcaagcctggggggtcccttagactctcctgtgcagcctctggattcaccttcactagatttactttgacctgggtccgccaggctccagggaaggggctggagtgggtctcatccattagtagtgggagtagtgacataaactacgcagactcagtgaagggccgattcaccatatccagagacaacgccaggaactccctgttcctgcaaatgagcagcctgagagtcgacgacacggctgtgtattactgtgcgaaagatccccggtcggggatctctggtcgctacgggatggacgtctggggccaagggaccacggtcatcgtctcctcagcttccaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagcca

SEQ ID NO: 4 - DNA, 540 bp, synthetic construct
  misc_feature 1..540: note = cDNA 25.6A HC variable region
  source 1..540: mol_type = other DNA
acctcggttcttcgattggctagcaccatggagacagacacactcctgctatgggtactgctgctctgggttccaggttccactggtgaccaggtgcagctgcaggagtcaggaggaggcctggtcaaggctggggggtccctgagactctcctgtgcagcctctggattcatgttcgagagatatagccttcactgggtccgtcagactccaggcaaggggctggagtgggtctcatccattagtagtcttagtggcagtcacataaactacgcagactcagtgaagggccgattcaccatctccagagacaacgccaagaattcactgtctctgcaaatgaacagcctgagagtcgaagacacggctatatattattgtgcgagagatcgacgttcggggagttcccccgtccccttggacgtctggggccaagggaccacggtcaccgtctcctctgcctccaccaagggcccatcggtcttccccctggcaccctcctccaagagcacctctgggggcacagcggccctgggctgc

SEQ ID NO: 5 - DNA, 855 bp, synthetic construct
  misc_feature 1..855: note = cDNA 36.1F HC variable region
  source 1..855: mol_type = other DNA
gtcactgccctcggttctatcgattggctagcaccatggagacagacacactcctgctatgggtactgctgctctgggttccaggttccactggtgaccaggtgcagctgcaggagtcgggcgcgggactggtgaagccttcggagaccctgtccctcacctgcgctgtctcaggtggacccttcagcggtgcctactggacgtggatccgccaaactccagggaaggggctggagtggattggagaggccggtcggagtggaaccaccaactacaatccgtccctcaagagtcgagtcaccatatcactggacacgtccaagagccagttttccctgaagctgacttccgtgaccgccgcggacacggctgtttacttctgtgggagacgccaaataatgtctttgagtaatctttataagagacccgttgactcttggggccggggaaccccggtcatcgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaa

SEQ ID NO: 6 - DNA, 874 bp, synthetic construct
  misc_feature 1..874: note = cDNA 36.9F HC variable region
  source 1..874: mol_type = other DNA
gtcactgccctcggttctatcgattggctagcaccatggagacagacacactcctgctatgggtactgctgctctgggttccaggttccactggtgacgaggtgcagctggtgcagtctggaggaggcctggtcaaggcgggggggtccctgaaactctcctgtggagcctctggattcaccttcagtagttatagcatgagctgggtccgccaggctccagggaaggggctggagtgggtctcatacattagtagtggtgggagttctatacactacgcagactcagtgaagggccgattcaccatctccagagacaacgccaagaattcactgtatctgcaaatgaagaacctgagggtcgacgacacgggtcggtattattgtgtgagagatccccgatcggggatctctggtcggtacggtatggacgtctggggtcaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaacccaaggacaccctcatgatc

SEQ ID NO: 7 - DNA, 859 bp, synthetic construct
  misc_feature 1..859: note = cDNA 37.2D HC variable region
  source 1..859: mol_type = other DNA
tcactgccctcggttctatcgattggctagcaccatggagacagacacactcctgctatgggtactgctgctctgggttccaggttccactggtgacgaagtgcagctggtgcagtctggagctgaggtgaagaagcctggggcttcagtgaaggtgtcctgcaaggcctctggttacacctttacgaaatacggaatcagctgggtgcgacaggcccctggacaagggcttgagtggatgggatggatcagcgcgtttaatggttacacaaggtatggtcagagattccagggcaaagtcaccatgaccacagacacatccacgaacacagcctctttggaggtgaggaccctgacatctaacgacacggccgtctattactgtgcgagacaatatcccgaccaatatagtagcagcggttggccccgcctcttcgccatggacgtctggggccaagggaccacggtcatcgtctccccagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttc

SEQ ID NO: 8 - DNA, 960 bp, synthetic construct
  misc_feature 1..960: note = cDNA 37.2G HC variable region
  source 1..960: mol_type = other DNA
tcactgccctcggttctatcgattggctagcaccatggagacagacacactcctgctatgggtactgctgctctgggttccaggttccactggtgacgaggtgcagctggtggagtctgggggaggcctggtcaagccgggggggtcccggagactctcctgtgctgcctctggattcaccttcagtagagataccatgacctgggtccgccaggctccagggaaggggctggagtgggtcgcatccataagtagtggtagcagtgacataaactacgcagactcagtgaagggccgattcaccatctccagagacaacggcaagaactcactgtatctgcacatgaacagcctgagagccgacgacacggctatatattactgtgcgagagatccccggtcgggaatctctggtcggtatggtatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgt

SEQ ID NO: 9 - DNA, 924 bp, synthetic construct
  misc_feature 1..924: note = cDNA 37.7H HC variable region
  source 1..924: mol_type = other DNA
gtcactgcacctcggttctatcgattggctagcaccatggagacagacacactcctgctatgggtactgctgctctgggttccaggttccactggtgacgaggtgcagctggtgcagtctggaggaggcctggtcaaggcgggggggtccctgaggctctcctgtgcagcctccggattcacattcagcacctacagtatgaactggatccgccaggctccagggaaggggctggagtgggtcgcttccattagtagtcgaagtggcagtcacataaactacgtagactcagtgaagggacgattcaccatctccagagacaacgccagggacttattgtatctgcaaatgaacagcctgagagtcgacgactcggctctctattactgtgcgagagatcgccgttcggggacttctcccctccccttggacgtctggggccaagggaccacggtcaccgtcttctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaa

SEQ ID NO: 10 - DNA, 911 bp, synthetic construct
  misc_feature 1..911: note = cDNA 8.9F HC variable region
  source 1..911: mol_type = other DNA
cctcggttctatcgattggctagcaccatggagacagacacactcctgctatgggtactgctgctctgggttccaggttccactggtgaccagggcaccttgagggagtctggtccaggactggtgaggccttcggagaccctgtccctcacctgcggtgtctctggttattccatcagtagtggttactactggggctggatccggcagcccccagggaaggggctggagtggattgggaatatctatcgtagtgggagcacctactacaacccgtccctcaagagtcgagtcaccgtctcaatagacacgtccaaaaaccagttctccctgaagttgaattctgtgaccgccgcagacacggccgtgtattactgtgcgagatcgggtataaaagtggctgacgactattactacgaaatggacgtctggggccaagggaccgacgactactcttacgctatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgat

SEQ ID NO: 11 - DNA, 881 bp, synthetic construct
  misc_feature 1..881: note = cDNA NE13 HC variable region
  source 1..881: mol_type = other DNA
actgcacctcggttctatcgattggctagcaccatggagacagacacactcctgctatgggtactgctgctctgggttccaggttccactggtgacgaggttcagctggtggagtctgggggaggcctggtcaagcctggggggtccctgagactctcctgtgtagcctctggattcaccttcagttcctatagcatgaactgggtccgccaggctccagggaaggggctggagtgggtctcatccattagtagtggtagtagttacatagagtacgcagactcagtgaagggccgactcaccatctccagagacaacgccaagaagtcactgtatctgcaactgaacagcctgagagccgaggacacggctgtgtattactgtgcgagacacacagctcgaatcgactcttaccacggtatggacgtctggggccaagggaccacagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccc

SEQ ID NO: 12 - DNA, 921 bp, synthetic construct
  misc_feature 1..921: note = cDNA 12.1F HC variable region
  source 1..921: mol_type = other DNA
gtcactgcacctcggttctatcgattggctagcaccatggagacagacacactcctgctatgggtactgctgctctgggttccaggttccactggtgaccaggtgcagctgcaggagtcgggcgcaggactgttgaagccttcggagaccctgtccctcagttgcactgtcgatggtgagtccttcaatggtttcttctggacgtggatccgccagcccccagggaagggtctggagtggattggagaaatcaatcatcttgcaagcaccggctacaacccgtccctcaagagtcgagtcaccatttcagtagacacgtccaagaaccagttctctttgaagttgacctctgtgaccgccgcggacacggctgtgtattactgtgcgagaggatacagctatggttttgcatggcccaactaccactatttggacgtctggggcaaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttncccccaaaacccaaggacaccctcatgatctcccggacccctgaggtcacatgcgtggtggtggacgtgagc

SEQ ID NO: 13 - DNA, 874 bp, synthetic construct
  misc_feature 1..874: note = cDNA 9.8A HC variable region
  source 1..874: mol_type = other DNA
ttctatcgatttggctagcaccatggagacagacacactcctgctatgggtactgctgctctgggttccaggttccactggtgacgaggtgcagctggtgcagtctggaggacgcttggtacagcctggggggtccctgagactctcctgtgtagcctctggattcacctttagcagccatgccatgagctgggtccgccaggctccagggaaggggctggagtgggtctcaggttttagtggtagtagtggtaccacaaagtacgcagactccgtgaagggccggttcaccatctccagagacaattccaagaaaacgctgtatctgcaaatgaacagcctgagagccgaggacacggccgtatattactgtgcgaaaggcttctccccatttcggggagtacaattcccctactttgactactggggccagggaacgctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaggacaccctcatgatctcccggaccc

SEQ ID NO: 14 - DNA, 898 bp, synthetic construct
  misc_feature 1..898: note = cDNA 18.5C HC variable region
  source 1..898: mol_type = other DNA
gtccactgcacctcggttctatcgattggctagcaccatggagacagacacactcctgctatgggtactgctgctctgggttccaggttccactggtgacgaggttcagctggtggagtctgggggaggcctggtcaggccgggggggtcccttagactctcctgtgcagccgctggattcactttcaagagttatagcatgaattgggtccgccaggctccagggaggggcctggagtgggtctcatctatcactagtggtggtagtaagacatactatgcagacgtagtgaagggccgattcaccgtctccagagacaacgccaagcagtcgctctatctgcaaatgaacagcctgagagccgaggacacggctatatacttctgtgcgagatccctacatagtaccagccagcctagctacatggacgtctggggcagaaagatcacggtcatcgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgc

SEQ ID NO: 15 - DNA, 903 bp, synthetic construct
  misc_feature 1..903: note = cDNA 8.11G HC variable region
  source 1..903: mol_type = other DNA
tgcacctcggttctatcgattggctagcaccatggagacagacacactcctgctatgggtactgctgctctgggttccaggttccactggtgaccaggtgcagctgcaggagtcgggtccaggactggtgaagccttcggagaccctgtccctcacctgcagtatttctggtgtgtccaccagaaattattattggagctggatccgccagtccccagggaagggactggagtggattggatatatctttaacattgggaccaccaactacaatccgtccctcaagagtcgactcaccatatctgtagacacgtcgaagaaccagttctccctgaagatcacctctgtgaccgctgcggacacggccgtctattactgtgcgagtggatttgagtacggtgactataccttcgactactggggccagggaaccccggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatcttccggacccctgaggtcacatgcgtggtggtggacgtgagcca

SEQ ID NO: 16 - DNA, 860 bp, synthetic construct
  misc_feature 1..860: note = cDNA 25.10C HC variable region
  source 1..860: mol_type = other DNA
ctcggttctatcgattggctagcaccatggagacagacacactcctgctatgggtactgctgctctgggttccaggttccactggtgaccaggtgcagctgcaggagtctgggggaggcctggtcaagcctggggggtccctgagactctcctgtacagcctctggattcaacttcaataaatataacatgaactgggtccgccaggctccagggaaggggctggagtgggtctcatccattagtgctcttagcacttacatctattatgcagactcgctgaagggccgattcaccgtctccagagacaacgccaagaactcactgtttctgcaaatgaacagcctgagagacgacgacacggctgtttattactgtgcgagagaaatacgacgtgccagtacctggtccgccgacctctggggccgtggcactctggtcactgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccctccaaacccaaggacaccctcatgatct

SEQ ID NO: 17 - DNA, 716 bp, synthetic construct
  misc_feature 1..716: note = cDNA 10.4B LC variable region
  source 1..716: mol_type = other DNA
agctgtgaccggcgcctacctgagatcaccggtgctagcaccatggagacagacacactcctgctatgggtactgctgctctgggttccaggttccactggtgacgaaattgtgttgacacagtctccatcctcactgtctgcgtctgtaggagacagagtcaccatcacttgtcgggcgagtcgggacatcaatacttatttaggttggtttcagcagagaccagggaaagcccctaagtccctgatctatggtgcatctaatttgcaaaatggggtcccatcaaggttcagcggcagtggatctgggacgtattttactctcaccatcaacggcctgcagactgaagactttgcgacttattattgccaacaatatagcatctacccgctcagtctcggcggagggaccaaggcggacatgaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcc

SEQ ID NO: 18 - DNA, 906 bp, synthetic construct
  misc_feature 1..906: note = cDNA 19.7E LC variable region
  source 1..906: mol_type = other DNA
tcagctgtgaccggcgcctacctgagatcaccggtgctagcaccatggagacagacacactcctgctatggctcctgctgctctgggttccaggttccactggtgacgaaattgtgttgacacagtctccttccaccctgtctgcatctgtgggagacagagtcaccatcacttgccgggccagtcagagtattaataattggttggcctggtatcaggagaaaccagggaaagcccctaagctcctgataaataaggcgtctagtttagaaagtggggtcccatcaaggttcagcggcagtggatctgggacagaattcactctcaccatcaccagcctgcagcctgatgattttgcaacttattactgccaacaatataatagtaattcgtggacgttcggccaagggaccaaggtggacatgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagctagctcgacatgataagatacattgatgagtttgggacaaccacaactagaatgcagtgaaaaaaatgctttatttgtgaaatttgtgatgctattgcttttattgtgaaatttgtgatgctattgctttatttgtaaccattataa

SEQ ID NO: 19 - DNA, 894 bp, synthetic construct
  misc_feature 1..894: note = cDNA 2.9D LC variable region
  source 1..894: mol_type = other DNA
actgcacctcggttctatcgattggctagcaccatgaagacagacacactcctgctatgggtactgctgctctgggttccaggttccactggtgacgacattgtgctgacccagtctccagactccctggctgtgtctctgggcgagagggccaccatcaactgcaagtccagccagagtgttttatacagctccaacaataagaactacttagcttggtaccagcagaagccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgcaagcttggccgccatggcccaacttgtttattgcagcttataatggttacaaataaagcaatagcatcacaaatttcacaaataaagcatttttttcactgcattctagttgtggtttgtccaactc

SEQ ID NO: 20 - DNA, 903 bp, synthetic construct
  misc_feature 1..903: note = cDNA 25.6A LC variable region
  source 1..903: mol_type = other DNA
ctcggttctatcgattggctagcaccatggagacagacacactcctgctatgggtactgctgctctgggttccaggttccactggtgacctgcctgtgctgactcagcctgcctccgtgtctgggtctcctggacagtcgatcaccatctcctgcactggaaccagcagtgacgttggtgcttataactatgtctcctggtaccaacagcacccaggcaaagcccccaaactcataatttatgaagtcaagattcggccgtcaggggtgtctaatcgtttctctggctccaagtctggcaacacggcctccctgaccatctctgggctccaggctgaggacgaggctgattatttttgcagctcatattcaaccaacagcccttgggtgttcggcggagggacgaaggtgaccgtcctacgtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatgagcggccgcaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 21 - DNA, 900 bp, synthetic construct
  misc_feature 1..900: note = cDNA 36.1F LC variable region
  source 1..900: mol_type = other DNA
tccaggtcactgcacctcggttctatcgattggctagcaccatggagacagacacactcctgctatgggtactgctgctctgggttccaggttccactggtgacgaaattgtgctgacacagtctccaggcaccctgtctttgtctccaggggaaagagccaccctctcctgcagggccagtcagagtgttactaaaaactacttagcctggtaccagcagaaacctggccaggctcccaccctcgtcatctatgatgcatccaccagggccagtggcatcccagacaggttcattggcagtgggtctgggacagacttcactctcaccatcagcagactggagcctgaagattttgcagtatattactgccaccagtatggcagctcacctccgtacacttttggccgggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgcaagcttggccgccatggcccaacttgtttattgcagcttataatggttacaaataaagcaatagcatcacaaatttcacaaataaagcatttttttcactgcattctagttgtgggttgtccaaactcatcaatgta

SEQ ID NO: 22 - DNA, 922 bp, synthetic construct
  misc_feature 1..922: note = cDNA 36.9F LC variable region
  source 1..922: mol_type = other DNA
aggtcactgcacctcggttctatcgattggctagcaccatggagacagacacactcctgctatgggtactgctgctctgggttccaggttccactggtgacgacatcgtgatgacccagtctccagactccctggctgtgtctctgggcgagagggccaccatcaactgcaagtccagccagactgttttgttcacctcctattacgtagcttggtatcaacaaaagccagggcagccgcctaagttgctcttttccggggcctcttctcgggaatccggggtccctgaccgattcagtgccggcgggtctgggacagatttctatctcaccatcaacagcctgcaggctgaagatgtggcagattactattgtcagcaatatcatactcctcctttcactttcggcggagggaccaagctggagatcagacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgcaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcggga

SEQ ID NO: 23 - DNA, 858 bp, synthetic construct
  misc_feature 1..858: note = cDNA 37.2D LC variable region
  source 1..858: mol_type = other DNA
tcactgcacctcggttctatcgattggctagcaccatggagacagacacactcctgctatgggtactgctgctctgggttccaggttccactggtgacgaaacgacactcacgcagtctccagccaccctgtctgtgtctccaggggaaacagccaccctctcctgcagggccagtcaaaatgttatcaacaacttagcctggtaccagcagaaacctggccaggctcccaggctcctcatttatggtgcatccaccagggccactggtatcccagccaggttcagtggcagtgggtctgggacagagttcactctcaccatcagcagcatgcagtctgaagattttgcagtttattactgtcagcaatataatgactggcctcgaagttttggccaggggaccaggctggacatcagacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgcaagcttggccgccatggcccaacttgtttattgcagcttataatggttacaaataaagcaatagcatcacaaatttcacaaataaagcatttttttcactgcattct

SEQ ID NO: 24 - DNA, 958 bp, synthetic construct
  misc_feature 1..958: note = cDNA 37.7G LC variable region
  source 1..958: mol_type = other DNA
tccaggtcactgccctcggttctatcgattggctagcaccatggagacagacacactcctgctatgggtactgctgctctgggttccaggttccactggtgacgacattgtgctgacccagtctccaggcaccctgtctttgtctccaggggaaagagccaccctctcctgcagggccagtcagagtgtgaacagcatcttcttagcctggtaccagcagaaacctggccaggctcccaggctcctcatctatggtgcatccagcagggccactggcatcccagacaggttcagtggcagtgggtctgggacagacttcactctcaccatcagcagactggagcctgaggattttgcagtgtattactgtcagcagtatcatagctcacctaagctcactttcggcggagggaccaaggtggagatcaaacgaactgtggctgcaccatctgtcttcatcttcccgccatctggtgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgcaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgggaattaattcggcgcagcaccatggcctgaaataacctc

SEQ ID NO: 25 - DNA, 1015 bp, synthetic construct
  misc_feature 1..1015: note = cDNA 37.7H LC variable region
  source 1..1015: mol_type = other DNA
tcactgcacctcggttctatcgattggctagcaccatggagacagacacactcctgctatgggtactgctgctctgggttccaggttccactggtgaccagtctgccctgactcagcctgcctccgtgtctgggtctcctggacagtcgatcaccatctcctgcactggaaccggcagtgacattggtggttataactttgtctcctggtaccaacagtatcccggcaaagcccccaaactcattatttatgaggtccgtattcgggcctcaggggtttccaatcgcttctctggctccaagtctggcaacacggcctccctgaccatctctggactccaggctgaggacgaggctgattattactgcaactcatattcaatccacagcccttgggtgttcggcggagggaccaagttgaccgtcctgcgtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtgggagtcccacagaagctacagctgccaggtcacgcatgaagggagcaccgtggagaagacagtggcccctacagaatgttcatgagcggccgcaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgggaattaattcggcgcagcaccatggcctgaaataccctctgaaagaggaacttggttaggtaccttctgaggcggaaagaaccatctgtggaatgtgtgtc

SEQ ID NO: 26 - DNA, 910 bp, synthetic construct
  misc_feature 1..910: note = cDNA 8.9F LC variable region
  source 1..910: mol_type = other DNA
cactgccctcggttctatcgattggctagcaccatggagacagacacactcctgctatgggtactgctgctctgggttccaggttccactggtgaccaggcagggctgactcagcctgcctccgtgtctgggtctcctggacagtcgatcaccatctcctgcactgcagccaacagtgacattggtgattttaactttgtctcctggtaccaacagcgcccagacaaagcccccaaactcatggtttatgaggtcagcagtcggccctcaggggtttctaatcgcttctctggctccaagtctggcaacacggcctccctgaccatctctgggctccaggctgaggacgaggctgattattactgcacctcatatacaagcagcagcacttttgtcttcggaactgggaccaaggtcaccgtcctaggtcagcccaaggccaaccccactgtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatgagcggccgcaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 27 - DNA, 908 bp, synthetic construct
  misc_feature 1..908: note = cDNA NE13 LC variable region
  source 1..908: mol_type = other DNA
ctcccaggtcactgcacctcggttctatcgattggctagcaccatggagacagacacactcctgctatgggtactgctgctctgggttccaggttccactggtgacgaaacgacactcacgcagtctccaggcaccctgtctttgtctccaggggaaagagccaccctctcctgcagggccagtcagagtgttagcagcacctacttagcctggtaccagcagaaacctggccagtctcccaggctcctcatttatggtgcatccagtagggccactggcatcccagacaggttcagtggcagtgggtctgggacacagttcactctcaccatcaacagactggagcctgaagattttgcagtgtattactgtcagcagtttggtagcccgtggacattcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgcaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtc

SEQ ID NO: 28 - DNA, 932 bp, synthetic construct
  misc_feature 1..932: note = cDNA 12.1F LC variable region
  source 1..932: mol_type = other DNA
gtcactgcacctcggttctatcgattggctagcaccatggagacagacacactcctgctatgggtactgctgctctgggttccaggttccactggtgacgaaacgacactcacgcagtctccagccaccctgtctttgtctccaggggagagagccaccctctcctgtagggccagtcagagtgttagcagctacttagcctggtaccaacacaaacctggccaggctcccaggctcctcatctatggtgcatcaaagagggccactggcatcccgtccaggttcagtggcagtgggtctgggacagacttcagtctcaccatcagcagcctagagcctgaagattttgcagtttactactgtcagcaccgaagcgactggcggactaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgcaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgggaaattaatcggcgcagcaccat

SEQ ID NO: 29 - DNA, 895 bp, synthetic construct
  misc_feature 1..895: note = cDNA 9.8A LC variable region
  source 1..895: mol_type = other DNA
ggttctatcgattggctagcaccatggagacagacacactcctgctatgggtactgctgctctgggttccaggttccactggtgacgacatcgtgatgacccagtctccttccaccctgtctgcatctgtaggagacagagtcaccatcacttgccgggccagtcagagtattgataggtggttggcctggtatcagcagaaaccagggaaagcccctaagctcctgatctatcaggcatctagtttagaaagaggggtcccatcaaggttcagcggcagtggatctgggacagaattcactctcaccatcagcagcctgcagcccgatgattttgcaacttattactgccaacagtataatggttaccctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgcaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcg

SEQ ID NO: 30 - DNA, 784 bp, synthetic construct
  misc_feature 1..784: note = cDNA 18.5C LC variable region
  source 1..784: mol_type = other DNA
tccaggtccactgcacctcggttctatcgattggctagcaccatggagacagacacactcctgctatgggtactgctgctctgggttccaggttccactggtgacgacatccagatgacccagtctccaggcaccctgtctttgtctccaggggaaagagccaccctctcctgcagggccagtcagagtgttatcagttactacgtagcctggtaccagcacaaaggtggccaggctcccaggctcctcatttatggtgcatccagcagggccactggcgtcccagacaggttcagtggcagtgggtctgggacagacttcactctcaccatcagcagcctggagcctgaagattttgcactgtattactgtcagtactatgggagctcacctctgtgggcgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgcaagcttggccgccatggccc

SEQ ID NO: 31 - DNA, 845 bp, synthetic construct
  misc_feature 1..845: note = cDNA 8.11G LC variable region
  source 1..845: mol_type = other DNA
cggttctatcgattggctagcaccatggagacagacacactcctgctatgggtactgctgctctgggttccaggttccactggtgacgaaattgtgctgactcagtctccagccaccctgtctgtgtctccagggggtagggcctccctctcctgccgggccagtcagagtattggcgacaagttatcctggtatcagcagaaacctgggcaggctcccaggctcgtcatctatggtgcatataccagggccactgatatctcacccaggttcagtggcagtaggtctgggacagacttcaatctcaccatcagcagaatgcagtctggagactttgcagtttatttctgtcagcagtatgaaaactggcctcggacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccgcaagcttggccgccatggcccaacttgtttattgcagcttataatggttacaaataaagcaatagcatcacaaatttcacaaataaagcatttttttcactgcatt

SEQ ID NO: 32 - DNA, 900 bp, synthetic construct
  misc_feature 1..900: note = cDNA 25.10C LC variable region
  source 1..900: mol_type = other DNA
cctcggttctatcgattggctagcaccatggagacagacacactcctgctatgggtactgctgctctgggttccaggttccactggtgacgacatccagatgacccagtctccatcctccctgtctgcatctgttggagacagagtcatcatcacttgccgggcaagtcagagcatcagcagctctttaaattggtatcagcagaaaccagggaaagcccctaagctcctgatctatgctgcagtcaatttggagactggggtcccgtcaaggttcagtggcagtggatttgggacagatttcactctcgccatcagcaatgtgcaacctgaagattttgcaacttactactgtcaacagagcgatactcggacttttggccgggggaccaagctggacgtcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagaagtgttaggcggccgcaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgggaatt

SEQ ID NO: 33 - protein, 203 aa, synthetic construct
  REGION 1..203: note = HC variable region
METDTLLLWVLLLWVPGSTGDQVQLVQSGGGVVQPGRSLRVSCVTSGFNFRAYGMHWVRQIPGKGLEWVADIWSAETNRHYADSVKGRFTISRDNSKSTLYLQMNSLRAEDTGVYFCAKARPGYDYVVDLWGQGTLVIVSSASTKGPSVFPLAPCSRSTSGGTAALGCLVKDYFPEPVTVSWNSGALTSGVHTFPAVLQSSGL

SEQ ID NO: 34 - protein, 258 aa, synthetic construct
  REGION 1..258: note = HC variable region
METDTLLLWVLLLWVPGSTGDEVQLVESGGGLVRPGGSLRLSCAASGFSFSSYSMHWVRHVPGKGLVWVSYINSDGSTKIYADSVKGRFSISRDNAKNKLYLQMDSLRVEDTAVYSCVRLVHYDWSPFVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPQSCDKTHTCPPCPAPELL

SEQ ID NO: 35 - protein, 294 aa, synthetic construct
  REGION 1..294: note = HC variable region
METDTLLLWVLLLWVPGSTGDEVQLVESGGGLVKPGGSLRLSCAASGFTFTRFTLTWVRQAPGKGLEWVSSISSGSSDINYADSVKGRFTISRDNARNSLFLQMSSLRVDDTAVYYCAKDPRSGISGRYGMDVWGQGTTVIVSSASTKGPSVFPLAPCSRSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVS

SEQ ID NO: 36 - protein, 171 aa, synthetic construct
  REGION 1..171: note = HC variable region
METDTLLLWVLLLWVPGSTGDQVQLQESGGGLVKAGGSLRLSCAASGFMFERYSLHWVRQTPGKGLEWVSSISSLSGSHINYADSVKGRFTISRDNAKNSLSLQMNSLRVEDTAIYYCARDRRSGSSPVPLDVWGQGTTVTVSSASTKGPSVFPLAPSSKSTSGGTAALGC

SEQ ID NO: 37 - protein, 274 aa, synthetic construct
  VARIANT 274: note = X can be any amino acid
  REGION 1..274: note = HC variable region
METDTLLLWVLLLWVPGSTGDQVQLQESGAGLVKPSETLSLTCAVSGGPFSGAYWTWIRQTPGKGLEWIGEAGRSGTTNYNPSLKSRVTISLDTSKSQFSLKLTSVTAADTAVYFCGRRQIMSLSNLYKRPVDSWGRGTPVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPX

SEQ ID NO: 38 - protein, 279 aa, synthetic construct
  REGION 1..279: note = HC variable region
METDTLLLWVLLLWVPGSTGDEVQLVQSGGGLVKAGGSLKLSCGASGFTFSSYSMSWVRQAPGKGLEWVSYISSGGSSIHYADSVKGRFTISRDNAKNSLYLQMKNLRVDDTGRYYCVRDPRSGISGRYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPNPRTPSS

SEQ ID NO: 39 - protein, 275 aa, synthetic construct
  REGION 1..275: note = HC variable region
METDTLLLWVLLLWVPGSTGDEVQLVQSGAEVKKPGASVKVSCKASGYTFTKYGISWVRQAPGQGLEWMGWISAFNGYTRYGQRFQGKVTMTTDTSTNTASLEVRTLTSNDTAVYYCARQYPDQYSSSGWPRLFAMDVWGQGTTVIVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLF

SEQ ID NO: 40 - protein, 309 aa, synthetic construct
  REGION 1..309: note = HC variable region
METDTLLLWVLLLWVPGSTGDEVQLVESGGGLVKPGGSRRLSCAASGFTFSRDTMTWVRQAPGKGLEWVASISSGSSDINYADSVKGRFTISRDNGKNSLYLHMNSLRADDTAIYYCARDPRSGISGRYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGV

SEQ ID NO: 41 - protein, 296 aa, synthetic construct
  REGION 1..296: note = HC variable region
METDTLLLWVLLLWVPGSTGDEVQLVQSGGGLVKAGGSLRLSCAASGFTFSTYSMNWIRQAPGKGLEWVASISSRSGSHINYVDSVKGRFTISRDNARDLLYLQMNSLRVDDSALYYCARDRRSGTSPLPLDVWGQGTTVTVFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHE

SEQ ID NO: 42 - protein, 295 aa, synthetic construct
  VARIANT 295: note = X can be any amino acid
  REGION 1..295: note = HC variable region
METDTLLLWVLLLWVPGSTGDQGTLRESGPGLVRPSETLSLTCGVSGYSISSGYYWGWIRQPPGKGLEWIGNIYRSGSTYYNPSLKSRVTVSIDTSKNQFSLKLNSVTAADTAVYYCARSGIKVADDYYYEMDVWGQGTDDYSYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMX

SEQ ID NO: 43 - protein, 283 aa, synthetic construct
  REGION 1..283: note = HC variable region
METDTLLLWVLLLWVPGSTGDEVQLVESGGGLVKPGGSLRLSCVASGFTFSSYSMNWVRQAPGKGLEWVSSISSGSSYIEYADSVKGRLTISRDNAKKSLYLQLNSLRAEDTAVYYCARHTARIDSYHGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTP

SEQ ID NO: 44 - protein, 295 aa, synthetic construct
  VARIANT 271: note = X can be any amino acid
  REGION 1..295: note = HC variable region
METDTLLLWVLLLWVPGSTGDQVQLQESGAGLLKPSETLSLSCTVDGESFNGFFWTWIRQPPGKGLEWIGEINHLASTGYNPSLKSRVTISVDTSKNQFSLKLTSVTAADTAVYYCARGYSYGFAWPNYHYLDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLXPPKPKDTLMISRTPEVTCVVVDVS

SEQ ID NO: 45 - protein, 283 aa, synthetic construct
  REGION 1..283: note = HC variable region
METDTLLLWVLLLWVPGSTGDEVQLVQSGGRLVQPGGSLRLSCVASGFTFSSHAMSWVRQAPGKGLEWVSGFSGSSGTTKYADSVKGRFTISRDNSKKTLYLQMNSLRAEDTAVYYCAKGFSPFRGVQFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPRTPSSPGP

SEQ ID NO: 46 - protein, 287 aa, synthetic construct
  REGION 1..287: note = HC variable region
METDTLLLWVLLLWVPGSTGDEVQLVESGGGLVRPGGSLRLSCAAAGFTFKSYSMNWVRQAPGRGLEWVSSITSGGSKTYYADVVKGRFTVSRDNAKQSLYLQMNSLRAEDTAIYFCARSLHSTSQPSYMDVWGRKITVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTC

SEQ ID NO: 47 - protein, 290 aa, synthetic construct
  REGION 1..290: note = HC variable region
METDTLLLWVLLLWVPGSTGDQVQLQESGPGLVKPSETLSLTCSISGVSTRNYYWSWIRQSPGKGLEWIGYIFNIGTTNYNPSLKSRLTISVDTSKNQFSLKITSVTAADTAVYYCASGFEYGDYTFDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMIFRTPEVTCVVVDVS

SEQ ID NO: 48 - protein, 276 aa, synthetic construct
  REGION 1..276: note = HC variable region
METDTLLLWVLLLWVPGSTGDQVQLQESGGGLVKPGGSLRLSCTASGFNFNKYNMNWVRQAPGKGLEWVSSISALSTYIYYADSLKGRFTVSRDNAKNSLFLQMNSLRDDDTAVYYCAREIRRASTWSADLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPNPRTPS

SEQ ID NO: 49 - protein, 225 aa, synthetic construct
  REGION 1..225: note = LC variable region
METDTLLLWVLLLWVPGSTGDEIVLTQSPSSLSASVGDRVTITCRASRDINTYLGWFQQRPGKAPKSLIYGASNLQNGVPSRFSGSGSGTYFTLTINGLQTEDFATYYCQQYSIYPLSLGGGTKADMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSP

SEQ ID NO: 50 - protein, 235 aa, synthetic construct
  REGION 1..235: note = LC variable region
METDTLLLWLLLLWVPGSTGDEIVLTQSPSTLSASVGDRVTITCRASQSINNWLAWYQEKPGKAPKLLINKASSLESGVPSRFSGSGSGTEFTLTITSLQPDDFATYYCQQYNSNSWTFGQGTKVDMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 241 aa, synthetic construct
  REGION 1..241: note = LC variable region
MKTDTLLLWVLLLWVPGSTGDDIVLTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 237 aa, synthetic construct
  REGION 1..237: note = LC variable region
METDTLLLWVLLLWVPGSTGDLPVLTQPASVSGSPGQSITISCTGTSSDVGAYNYVSWYQQHPGKAPKLIIYEVKIRPSGVSNRFSGSKSGNTASLTISGLQAEDEADYFCSSYSTNSPWVFGGGTKVTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 53 - protein, 237 aa, synthetic construct
  REGION 1..237: note = LC variable region
METDTLLLWVLLLWVPGSTGDEIVLTQSPGTLSLSPGERATLSCRASQSVTKNYLAWYQQKPGQAPTLVIYDASTRASGIPDRFIGSGSGTDFTLTISRLEPEDFAVYYCHQYGSSPPYTFGRGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 238 aa, synthetic construct
  REGION 1..238: note = LC variable region
METDTLLLWVLLLWVPGSTGDDIVMTQSPDSLAVSLGERATINCKSSQTVLFTSYYVAWYQQKPGQPPKLLFSGASSRESGVPDRFSAGGSGTDFYLTINSLQAEDVADYYCQQYHTPPFTFGGGTKLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 235 aa, synthetic construct
  REGION 1..235: note = LC variable region
METDTLLLWVLLLWVPGSTGDETTLTQSPATLSVSPGETATLSCRASQNVINNLAWYQQKPGQAPRLLIYGASTRATGIPARFSGSGSGTEFTLTISSMQSEDFAVYYCQQYNDWPRSFGQGTRLDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 237 aa, synthetic construct
  REGION 1..237: note = LC variable region
METDTLLLWVLLLWVPGSTGDDIVLTQSPGTLSLSPGERATLSCRASQSVNSIFLAWYQQKPGQAPRLLIYGASSRATGIPDRFSGSGSGTDFTLTISRLEPEDFAVYYCQQYHSSPKLTFGGGTKVEIKRTVAAPSVFIFPPSGEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 237 aa, synthetic construct
  REGION 1..237: note = LC variable region
METDTLLLWVLLLWVPGSTGDQSALTQPASVSGSPGQSITISCTGTGSDIGGYNFVSWYQQYPGKAPKLIIYEVRIRASGVSNRFSGSKSGNTASLTISGLQAEDEADYYCNSYSIHSPWVFGGGTKLTVLRQPKAAPSVTLFPPSSEELQANKATLVCLISDFYPGAVTVAWKADSSPVKAGVETTTPSKQSNNKYAASSYLSLTPEQWESHRSYSCQVTHEGSTVEKTVAPTECS

SEQ ID NO: 58 - protein, 237 aa, synthetic construct
  REGION 1..237: note = LC variable region
METDTLLLWVLLLWVPGSTGDQAGLTQPASVSGSPGQSITISCTAANSDIGDFNFVSWYQQRPDKAPKLMVYEVSSRPSGVSNRFSGSKSGNTASLTISGLQAEDEADYYCTSYTSSSTFVFGTGTKVTVLGQPKANPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 59 - protein, 236 aa, synthetic construct
  REGION 1..236: note = LC variable region
TMETDTLLLWVLLLWVPGSTGDETTLTQSPGTLSLSPGERATLSCRASQSVSSTYLAWYQQKPGQSPRLLIYGASSRATGIPDRFSGSGSGTQFTLTINRLEPEDFAVYYCQQFGSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 235 aa, synthetic construct
  REGION 1..235: note = LC variable region
METDTLLLWVLLLWVPGSTGDETTLTQSPATLSLSPGERATLSCRASQSVSSYLAWYQHKPGQAPRLLIYGASKRATGIPSRFSGSGSGTDFSLTISSLEPEDFAVYYCQHRSDWRTTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 235 aa, synthetic construct
  REGION 1..235: note = LC variable region
METDTLLLWVLLLWVPGSTGDDIVMTQSPSTLSASVGDRVTITCRASQSIDRWLAWYQQKPGKAPKLLIYQASSLERGVPSRFSGSGSGTEFTLTISSLQPDDFATYYCQQYNGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 237 aa, synthetic construct
  REGION 1..237: note = LC variable region
METDTLLLWVLLLWVPGSTGDDIQMTQSPGTLSLSPGERATLSCRASQSVISYYVAWYQHKGGQAPRLLIYGASSRATGVPDRFSGSGSGTDFTLTISSLEPEDFALYYCQYYGSSPLWAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 235 aa, synthetic construct
  REGION 1..235: note = LC variable region
METDTLLLWVLLLWVPGSTGDEIVLTQSPATLSVSPGGRASLSCRASQSIGDKLSWYQQKPGQAPRLVIYGAYTRATDISPRFSGSRSGTDFNLTISRMQSGDFAVYFCQQYENWPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 291 aa, synthetic construct
  REGION 1..291: note = LC variable region
METDTLLLWVLLLWVPGSTGDDIQMTQSPSSLSASVGDRVIITCRASQSISSSLNWYQQKPGKAPKLLIYAAVNLETGVPSRFSGSGFGTDFTLAISNVQPEDFATYYCQQSDTRTFGRGTKLDVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEVLGGRKLGRHGPTCLLQLIMVTNKAIASQISQIKHFFHCILVVVCPNSSMYLIMSGSGI

SEQ ID NO: 65 - protein, 8 aa, Homo sapiens
GFNFRAYG

SEQ ID NO: 66 - protein, 9 aa, Homo sapiens
IWSAETNRH

SEQ ID NO: 67 - protein, 13 aa, Homo sapiens
AKARPGYDYVVDL

SEQ ID NO: 68 - protein, 8 aa, Homo sapiens
GFSFSSYS

SEQ ID NO: 69 - protein, 9 aa, Homo sapiens
INSDGSTKI

SEQ ID NO: 70 - protein, 12 aa, Homo sapiens
VRLVHYDWSPFV

SEQ ID NO: 71 - protein, 8 aa, Homo sapiens
GFTFTRFT

SEQ ID NO: 72 - protein, 9 aa, Homo sapiens
ISSGSSDIN

SEQ ID NO: 73 - protein, 16 aa, Homo sapiens
AKDPRSGISGRYGMDV

SEQ ID NO: 74 - protein, 8 aa, Homo sapiens
GFMFERYS

SEQ ID NO: 75 - protein, 10 aa, Homo sapiens
ISSLSGSHIN

SEQ ID NO: 76 - protein, 15 aa, Homo sapiens
ARDRRSGSSPVPLDV

SEQ ID NO: 77 - protein, 8 aa, Homo sapiens
GGPFSGAY

SEQ ID NO: 78 - protein, 8 aa, Homo sapiens
AGRSGTTN

SEQ ID NO: 79 - protein, 18 aa, Homo sapiens
GRRQIMSLSNLYKRPVDS

SEQ ID NO: 80 - protein, 8 aa, Homo sapiens
GFTFSSYS

SEQ ID NO: 81 - protein, 9 aa, Homo sapiens
ISSGGSSIH

SEQ ID NO: 82 - protein, 16 aa, Homo sapiens
VRDPRSGISGRYGMDV

SEQ ID NO: 83 - protein, 8 aa, Homo sapiens
GYTFTKYG

SEQ ID NO: 84 - protein, 9 aa, Homo sapiens
ISAFNGYTR

SEQ ID NO: 85 - protein, 21 aa, Homo sapiens
ARQYPDQYSSSGWPRLFAMDV

SEQ ID NO: 86 - protein, 8 aa, Homo sapiens
GFTFSRDT

SEQ ID NO: 87 - protein, 9 aa, Homo sapiens
ISSGSSDIN

SEQ ID NO: 88 - protein, 16 aa, Homo sapiens
ARDPRSGISGRYGMDV

SEQ ID NO: 89 - protein, 8 aa, Homo sapiens
GFTFSTYS

SEQ ID NO: 90 - protein, 10 aa, Homo sapiens
ISSRSGSHIN

SEQ ID NO: 91 - protein, 15 aa, Homo sapiens
ARDRRSGTSPLPLDV

SEQ ID NO: 92 - protein, 9 aa, Homo sapiens
GYSISSGYY

SEQ ID NO: 93 - protein, 8 aa, Homo sapiens
IYRSGSTY

SEQ ID NO: 94 - protein, 31 aa, Homo sapiens
ARSGIKVADDYYYEMDVWGQGTDDYSYAMDV

SEQ ID NO: 95 - protein, 8 aa, Homo sapiens
GFTFSSYS

SEQ ID NO: 96 - protein, 9 aa, Homo sapiens
ISSGSSYIE

SEQ ID NO: 97 - protein, 15 aa, Homo sapiens
ARHTARIDSYHGMDV

SEQ ID NO: 98 - protein, 8 aa, Homo sapiens
GESFNGFF

SEQ ID NO: 99 - protein, 8 aa, Homo sapiens
INHLASTG

SEQ ID NO: 100 - protein, 18 aa, Homo sapiens
ARGYSYGFAWPNYHYLDV

SEQ ID NO: 101 - protein, 8 aa, Homo sapiens
GFTFSSHA

SEQ ID NO: 102 - protein, 9 aa, Homo sapiens
FSGSSGTTK

SEQ ID NO: 103 - protein, 17 aa, Homo sapiens
AKGFSPFRGVQFPYFDY

SEQ ID NO: 104 - protein, 8 aa, Homo sapiens
GFTFKSYS

SEQ ID NO: 105 - protein, 9 aa, Homo sapiens
ITSGGSKTY

SEQ ID NO: 106 - protein, 15 aa, Homo sapiens
ARSLHSTSQPSYMDV

SEQ ID NO: 107 - protein, 8 aa, Homo sapiens
GVSTRNYY

SEQ ID NO: 108 - protein, 8 aa, Homo sapiens
IFNIGTTN

SEQ ID NO: 109 - protein, 13 aa, Homo sapiens
ASGFEYGDYTFDY

SEQ ID NO: 110 - protein, 8 aa, Homo sapiens
GFNFNKYN

SEQ ID NO: 111 - protein, 9 aa, Homo sapiens
ISALSTYIY

SEQ ID NO: 112 - protein, 14 aa, Homo sapiens
AREIRRASTWSADL

SEQ ID NO: 113 - protein, 6 aa, Homo sapiens
RDINTY

SEQ ID NO: 114 - protein, 9 aa, Homo sapiens
QQYSIYPLS

SEQ ID NO: 115 - protein, 6 aa, Homo sapiens
QSINNW

SEQ ID NO: 116 - protein, 9 aa, Homo sapiens
QQYNSNSWT

SEQ ID NO: 117 - protein, 12 aa, Homo sapiens
QSVLYSSNNKNY

SEQ ID NO: 118 - protein, 9 aa, Homo sapiens
QQYYSTPPT

SEQ ID NO: 119 - protein, 9 aa, Homo sapiens
SSDVGAYNY

SEQ ID NO: 120 - protein, 10 aa, Homo sapiens
SSYSTNSPWV

SEQ ID NO: 121 - protein, 7 aa, Homo sapiens
QSVTKNY

SEQ ID NO: 122 - protein, 10 aa, Homo sapiens
HQYGSSPPYT

SEQ ID NO: 123 - protein, 9 aa, Homo sapiens
QTVLFTSYY

SEQ ID NO: 124 - protein, 9 aa, Homo sapiens
QQYHTPPFT

SEQ ID NO: 125 - protein, 6 aa, Homo sapiens
QNVINN

SEQ ID NO: 126 - protein, 9 aa, Homo sapiens
QQYNDWPRS

SEQ ID NO: 127 - protein, 7 aa, Homo sapiens
QSVNSIF

SEQ ID NO: 128 - protein, 10 aa, Homo sapiens
QQYHSSPKLT

SEQ ID NO: 129 - protein, 9 aa, Homo sapiens
GSDIGGYNF

SEQ ID NO: 130 - protein, 10 aa, Homo sapiens
NSYSIHSPWV

SEQ ID NO: 131 - protein, 9 aa, Homo sapiens
NSDIGDFNF

SEQ ID NO: 132 - protein, 10 aa, Homo sapiens
TSYTSSSTFV

SEQ ID NO: 133 - protein, 7 aa, Homo sapiens
QSVSSTY

SEQ ID NO: 134 - protein, 8 aa, Homo sapiens
QQFGSPWT

SEQ ID NO: 135 - protein, 6 aa, Homo sapiens
QSVSSY

SEQ ID NO: 136 - protein, 9 aa, Homo sapiens
QHRSDWRTT

SEQ ID NO: 137 - protein, 6 aa, Homo sapiens
QSIDRW

SEQ ID NO: 138 - protein, 9 aa, Homo sapiens
QQYNGYPLT

SEQ ID NO: 139 - protein, 7 aa, Homo sapiens
QSVISYY

SEQ ID NO: 140 - protein, 10 aa, Homo sapiens
QYYGSSPLWA

SEQ ID NO: 141 - protein, 6 aa, Homo sapiens
QSIGDK

SEQ ID NO: 142 - protein, 9 aa, Homo sapiens
QQYENWPRT

SEQ ID NO: 143 - protein, 6 aa, Homo sapiens
QSISSS

SEQ ID NO: 144 - protein, 7 aa, Homo sapiens
QQSDTRT